(19) United States
(12) Patent Application Publication
(10) Pub. No.: US 2016/0214958 A1
(43) Pub. Date: Jul. 28, 2016

(54) COMPOUNDS AND USE FOR TREATING CANCER

(71) Applicant: GLIONOVA AB, Stockholm (SE)

(72) Inventors: Katarina FÄRNEGÅRDH, EKER (SE); Ylva GRAVENFORS, SÖDERTÄLJE (SE); Patrik ERNFORS, Bromma (SE); Lars HAMMARSTRÖM, Sollentuna (SE); Satish KITAMBI, Huddinge (SE)

(21) Appl. No.: 14/916,967

(22) PCT Filed: Sep. 9, 2014
(86) PCT No.: PCT/IB2014/002636

§ 371 Date: Mar. 4, 2016

Related U.S. Application Data
(60) Provisional application No. 62/014,163, filed on Jun. 19, 2014.
(60) Provisional application No. 61/917,581, filed on Dec. 18, 2013.
(60) Provisional application No. 61/875,420, filed on Sep. 9, 2013.

(30) Foreign Application Priority Data
Sep. 9, 2013 (SE) SE1351041-7

(51) Int. Cl. C07D 401/06; A61K 47/48; G01N 33/50; A61K 49/00

(52) U.S. Cl. CPC C07D 401/06; A61K 49/0008; A61K 47/48061; G01N 33/5088

(57) ABSTRACT

The present invention relates to certain 2,4-disubstituted quinoline derivatives, to their therapy, as well as to pharmaceutical compositions comprising said compounds. More specifically the invention relates to certain 2,4-disubstituted quinoline derivatives or pharmaceutical compositions comprising said compounds for the treatment of cancers characterized by overactive Ras and/or Rac or signalling pathway.

### CROSS REFERENCE TO RELATED APPLICATIONS

This application claims the benefit of priority under 35 U.S.C. §119 to Swedish application SE1351041-7, filed Sep. 9, 2013, U.S. Provisional Patent Application Ser. No. 61/875,420, filed on Sep. 9, 2013, U.S. Provisional Patent Application Ser. No. 61/917,581, filed on Dec. 18, 2013, and U.S. Provisional Patent Application Ser. No. 62/014,163, filed on Jun. 19, 2014, the entire disclosures of which are incorporated by reference herein.

### FIELD OF THE INVENTION

The present invention relates to certain 2,4-disubstituted quinoline derivatives, their use in therapy, as well as to pharmaceutical compositions comprising said compounds. Specifically, the invention relates to certain 2,4-disubstituted quinoline derivatives and pharmaceutical compositions comprising these compounds for the treatment of cancer. The invention further relates to assays for identifying such compounds. The invention also relates to the use of cancer-cell specific non-clathrin-dependent vacuolization for compound delivery and/or imaging methods.

### BACKGROUND OF THE INVENTION

A glioma is a type of tumor that starts in the brain or spine, which arises from glial cells. Most gliomas are intracranial tumors, which affect roughly 7 of 100,000 individuals annually making it the most common form of brain cancer. Gliomas are classified by cell type, by grade, and by location. Gliomas are named according to the specific type of cell they share histological features with. The main types of gliomas are Ependymomas (ependymal cells), Astrocytomas (astrocytes), Oligodendrogliomas (oligodendrocytes) and mixed gliomas (containing cells from different types of glia). Gliomas are further categorized according to their grade, which is determined by the pathologic evaluation of the tumor. According to the WHO (World Health Organization) gliomas are graded from I to IV, in which grade I is the least advanced disease (best prognosis) and grade IV the most advanced disease (worst prognosis). Grade I gliomas (e.g. angiocentric glioma, pilocytic astrocytoma, papillary glioneuronal tumors (PGNT), pituicytoma) are relatively benign with slow proliferation rates and the possibility of cure following surgical resection alone. Grade II tumors (e.g. oligodendroglioma, extraventricular neurocytoma, oligoastrocytoma and astrocytoma) are similarly slowly proliferating, but unlike pilocytic astrocytoma are prone to malignant progression through slow infiltration of neighboring tissue and can progress to higher grades of malignancy. Grade III lesions (e.g. anaplastic astrocytoma, anaplastic oligoastrocytoma, and anaplastic ganglioglioma) have histological evidence of malignancy and require both surgical resectioning and subsequent chemotherapy. The WHO grade IV designation (e.g. glioblastoma, embryonal neoplasms, gliosarcomas) are highly malignant, mitotically active tumors associated with rapid disease progression and invariably fatal outcome. Gliomas can also be classified according to their location, whether they are above or below the tentorium membrane in the brain. The tumors are either supratentorial (above the tentorium), infratentorial (below the tentorium) or pontine (located in the pons of the brainstem).

Glioblastoma multiforme (GBM or grade IV astrocytoma) is the most common and aggressive glioma and is characterized by high proliferative rate, aggressive invasiveness and resistance to radio- and chemotherapy. Despite improvements in treatment strategies involving chemo-irradiation approach that results in a significant increase in survival, due to tumor recurrence the median survival time is still limited to approximately 15 months. Thus, new therapies are needed, and understanding the biology behind tumor development is of great importance in finding new efficient treatments to enhance patient survival.

Tumor development involves somatic, and sometimes inherited, mutations that can either be gain-of-function mutations in proto-oncogenes or loss-of-function mutations in tumor suppressor genes that lead to fundamental changes in the biology of the cell, resulting in cancer. Such alterations often involve enhanced transduction of mitogentic signals or regulators of the cell cycle, apoptosis, senescence, cell adhesion or DNA repair pathways. Genomic studies of hundreds of glioblastoma multiforme (GBM) samples have led to a comprehensive insight into the genomic landscape of GBM and reveal both gain and loss of function in core signaling pathways commonly activated, including the Receptor tyrosine kinase (RTK/RAS) oncogenic pathway with alterations in EGFR/PI3K/PTEN/NF1/RAS; the p53 pathway with changes in TP53/MDM2/MDM4/p14ARF changes; and finally the cell-cycle regulatory pathway, with alterations in RB1/CDK4/p16NK4A/CDKN2B with most GBM tumors having genetic alteration in all three pathways. The consequence is a fueling of cell proliferation and enhanced survival and invasion properties, while preventing tumor cells from senescence, apoptosis and activation of cell cycle checkpoints. Consistently, malignant gliomas are among the most aggressive human cancers and represent the majority of malignant tumors in the CNS. GBM is essentially incurable even when aggressive therapies based on surgical tumor resection and concomitant chemotherapy and radiotherapy are implemented and only 3-5% of patients survive longer than 3 years due to disease recurrence.

Although frequently present in small numbers, cancer stem cells (CSCs) have the ability to originate tumors when xenotransplanted into animals, whereas the remaining non-CSC tumor mass most often cannot. The small population of GBM cells with stem/progenitor cell characteristics referred to as cancer stem cells can seed growth of new tumors and are believed to be the main driver of malignancy, metastasis and tumor recurrence, promoting resistance against radiation-based therapy and chemotherapy. The current golden standard chemotherapy used in treating gliomas is temozolomide (TMZ), an anti-neoplastic primarily targeting DNA replication. TMZ is associated with severe side effects and limited efficacy in targeting CSCs. The tumor-initiating CSCs are believed to be relatively quiescent, which could contribute to disease recurrence following current therapeutic strategies targeting intracellular processes associated with cell division (e.g. TMZ). Tumor initiating cells with CSC properties have been identified in glioblastoma with high tumorigenic potential and a low proliferation rate and present some phenotypical similarities with normal stem cells, such as the CD133 gene expression and other genes commonly expressed in neural stem cells. CSCs have been shown to differentiate into astrocytes, oligodendrocytes and neurons, as well as disperse into new locations of the brain.

Unlike several other forms of cancer where identification of participating gene products by genetic studies have resulted in a series of drugs neutralizing the function gained by the genetic alterations, the complexity and diversity of glioblastoma genetics has prevented a simple strategy for therapeutic targeting. The new approaches focused on neutralizing abnormalities underlying tumor development have only had limited success to date.

Cancers in the nervous system are highly diverse, of different cellular origin, different genetic background, and appearing at different times in life by different mechanisms. Neurological tumors includes everything from peripheral tumors such as, various nerve sheet tumors, neurofibroma (neurofibrosarcoma, neurofibromatosis), neurilemmoma/schwannoma (acoustic neuroma, neuroblastoma, spinal cord and brain tumors such as meningioma, hemangiopericytoma, primary CNS lymphoma, ependymoma, choroid plexus tumor, ganglioneuroma, retinoblastoma, neurocytoma, medulloblastoma, medulloepithelioma, glioma, oligodendroglioma.

In brain cancer, such as for instance glioblastoma, PRC2 activity is inhibited rather than increased (Lewis, P W (2013) Science, 240, 857-861). In glioma, RNAi-mediated attenuation or pharmacological inhibition of PRC2 activity has little to no effect on apoptosis or BrdU incorporation, but changes gene expression (Natsume A, (2013) Cancer Res, 73, 4559; Chan, K.-M. et al. (2013) Genes Dev. 27, 985-90). Such reduced PRC2 activity underlies a depression resulting in elevated expression of genes that, when expressed, are known drivers of glioma. Furthermore, mislocalized PRC2 in the genome of glioma cells also leads to increased gene expression of some genes, including known tumor suppressors (Chan, K.-M. et al. (2013) Genes Dev. 27, 985-90). Therefore, reduced PRC2 activity in glioma would be expected to fuel cancer.

International patent application PCT/CA2012/050767 (WO/2013/059944) discloses compounds of the general formula

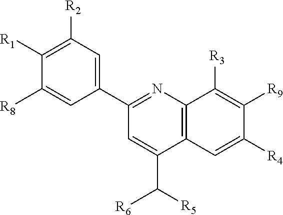

for the treatment of diseases associated with a hyperactive polycomb 2 complex (PRC2), including various cancer diseases. The experimental data provided are for lymphoma cell lines and breast cancer cell lines. No evidence is presented for any type of nervous system cancer. Further, glioma is not considered a disease associated with a hyperactive polycomb 2 complex (PRC2).

Further, α-2-piperidyl-2-phenyl-4-quinolinemethanol was much more effective against avian malaria than the corresponding compound without the 2-phenyl group suggesting the synthesis of analogous compounds containing different 2-aryl substituents. Journal of the American Chemical Society (1946), 68, 2705-8. None of these compounds have any relation to cancer.

Small molecular inhibitors, including 2-(4-chlorophenyl)-quinoline-4-yl)-(piperidin-2-yl)methanol and piperidin-2-yl(2-(4-(trifluoromethyl)phenyl)quinolin-4-yl)methanol, of biofilm formation in Vibrio cholera are disclosed in Molecular BioSystems (2011), 7(4), 1176-1184, and Organic Letters (2013), 15(6), 1234-1237.

International patent application PCT/US2013/027276 (WO/2013/126664) and Journal of Medicinal Chemistry (2012), 55, 3113-3121, disclose the use of optically active stereoisomers of the compound (2-(4-methoxyphenyl)quinolin-4-yl)-(piperidin-2-yl)methanol (NSC23925) to reverse multidrug resistance in human cancers. The disclosures relate to targeting the function of the P-glycoprotein (Pgp) MDR1 transporter complex in combination with other chemotherapeutics and claims no antineoplastic effect of the compound itself.

There is a continued need to develop novel glioma therapies, including those with unique mechanisms of action, which can improve the current very poor prognosis for glioma cancer patients.

### SUMMARY OF THE INVENTION

The present invention relates to new compounds, certain 2,4-disubstituted quinoline derivatives, to their use in therapy, as well as to a pharmaceutical composition comprising said compounds. More specifically the invention relates to certain 2,4-disubstituted quinoline derivatives or pharmaceutical compositions comprising said compounds for the treatment of cancers associated with altered Ras/Rac activity. Even more specifically, the invention relates to certain 2,4-disubstituted quinoline derivatives or pharmaceutical compositions comprising said compounds for the treatment of glioma. The invention further relates to assays for identifying such compounds. The present invention aims at providing molecules capable of selectively killing tumor cells with minimal effects on other cell types of the body.

Tumor-initiating cancer cells, similar to other stem-like cells, have unique molecular features that may allow for selective targeting of cancer, and for treatment of cancer, specifically cancers associated with altered Ras/Rac activity, such as gliomas, and more specifically glioblastoma (also referred to herein as glioblastoma multiforme, or GBM). The present invention relates to providing compounds capable of selectively killing tumor cells and/or cancer stem cells with minimal effects on other cell types of the body. More specifically, the invention relates to the preparation and use of 2,4-disubstituted quinoline derivatives in the treatment of cancers associated with altered Ras/Rac activity, such as, but not limited to, pancreatic, lung, thyroid, urinary tract, colorectal, salivary, prostate, intestinal, skin, hematological/lymphoid malignancies, gliomas and cervical cancer.

Further, the invention also relates to uses of a new non-clathrin-dependent vacuolization cell death mechanism selective for cancers with altered Ras/Rac activity and/or downstream signaling pathway and specifically glioma cells, in particular glioblastoma cells. The selective vacuolization may be used, e.g., for delivery of desired compounds or substances selectively to cancer cells, specifically glioma cells, in particular glioblastoma cells, or for the delivery of imaging molecules for use in selective imaging of cancer cells, specifically glioma cells, in particular glioblastoma cells. The compounds of the invention may be used to achieve this selective vacuolization, or any other suitable compound inducing said same selective vacuolization mechanism in cancer cells, specifically, glioma cells, in particular glioblastoma cells. Moreover, the invention also relates to a novel zebrafish screening assay for identifying such compounds effective in the treatment of cancer, specifically gliomas, in particular glioblastomas, and/or compounds inducing said cancer, specifically glioma cell, in particular glioblastoma cell-specific vacuolization.

One aspect of the present invention is a compound of formula (I)

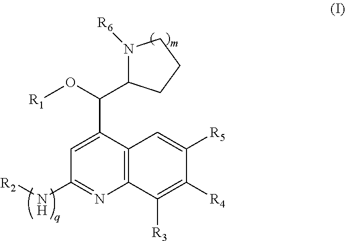

including stereoisomers and tautomers thereof, wherein

m is 1, 2 or 3;

q is 0 or 1;

R₁ is H or C1-C3 alkyl;

R₂ is selected from C1-C6 alkyl; and C3-C10 unsaturated or saturated, mono- or polycyclic carbocyclyl, heterocyclyl, and heteroaryl, each optionally substituted with one or more radicals R₇;

R₃, R₄ and R₅ are independently selected from H, halogen and C1-C6 alkyl optionally substituted with one or more halogens; or

R₃ and R₄, together with the adjacent atoms to which they are attached, form a benzene ring, and

R₅ is selected from H, halogen and C1-C6 alkyl optionally substituted with one or more halogens;

R₆ is H or C1-C3 alkyl;

each R₇ is independently selected from C1-C6 alkoxy, C1-C6 alkyl, C1-C6 alkynyl, C1-C6 alkenyl, halogen, alkylamino and NR₈C(O)OR₉;

R₈ is selected from H and C1-C3 alkyl; and

R₉ is C1-C6 alkyl, heteroaromatic or phenyl;

or a pharmaceutically acceptable salt, solvate or prodrug of the compound(s) of the formula (I), for use in the treatment of cancers associated with altered Ras/Rac activity. For example, the compound of formula I is not mefloquine. For example, R2 is not unsubstituted pyridyl.

For example, the invention relates to a compound of formula I selected from compounds S8, S9, S14, S16, S19, S20, S21, S22, and S23.

For example, the invention relates to a compound of formula I selected from compounds S24, S25, S26, S27, S28, and S29.

In some embodiments, the compound of the invention is a compound of formula I wherein m is 1 or 2.

In some embodiments, the compound of the invention is a compound of formula I wherein q is 0.

In some embodiments, the compound of the invention is a compound of formula I wherein m is 2 and q is 0.

In some embodiments, the compound of the invention is a compound of formula I wherein R2 is C6-C10 unsaturated or saturated, mono- or polycyclic carbocyclyl.

In some embodiments, the compound of the invention is a compound of formula I wherein R2 is phenyl.

Another aspect of the invention is a compound, selected from
tert-butyl 4-(4-(hydroxy(piperidin-2-yl)methyl)quinolin-2-yl)benzyl(methyl)carbamate,
2-(4-chlorophenyl)-4-(methoxy(piperidin-2-yl)methyl)quinoline,
(2-(4-chlorophenyl)quinolin-4-yl)(pyrrolidin-2-yl)methanol,
(2-(4-ethynylphenyl)quinolin-4-yl)(piperidin-2-yl)methanol,
(2-(4-chlorophenyl)quinolin-4-yl)(1-methylpiperidin-2-yl)methanol,
(R)-(2-(4-chlorophenyl)quinolin-4-yl)((S)-piperidin-2-yl)methanol,
(S)-(2-(4-chlorophenyl)quinolin-4-yl)((R)-piperidin-2-yl)methanol,
(S)-(2-(4-chlorophenyl)quinolin-4-yl)((S)-piperidin-2-yl)methanol, and
(R)-(2-(4-chlorophenyl)quinolin-4-yl)((R)-piperidin-2-yl)methanol or a pharmaceutically acceptable salt, solvate or prodrug thereof.

Still another aspect is a compound selected from
tert-butyl 4-(4-(hydroxy(piperidin-2-yl)methyl)quinolin-2-yl)benzyl(methyl)carbamate,
2-(4-chlorophenyl)-4-(methoxy(piperidin-2-yl)methyl)quinoline,
(2-(4-chlorophenyl)quinolin-4-yl)(pyrrolidin-2-yl)methanol,
(2-(4-ethynylphenyl)quinolin-4-yl)(piperidin-2-yl)methanol
(2-(4-chlorophenyl)quinolin-4-yl)(1-methylpiperidin-2-yl)methanol,
(R)-(2-(4-chlorophenyl)quinolin-4-yl)((S)-piperidin-2-yl)methanol,
(S)-(2-(4-chlorophenyl)quinolin-4-yl)((R)-piperidin-2-yl)methanol,
(S)-(2-(4-chlorophenyl)quinolin-4-yl)((S)-piperidin-2-yl)methanol, and
(R)-(2-(4-chlorophenyl)quinolin-4-yl)((R)-piperidin-2-yl)methanol
   or a pharmaceutically acceptable salt, solvate or prodrug thereof, for use in therapy of cancers associated with altered Ras/Rac activity.

Still another aspect is a compound selected from
tert-butyl 4-(4-(hydroxy(piperidin-2-yl)methyl)quinolin-2-yl)benzyl(methyl)carbamate,
2-(4-chlorophenyl)-4-(methoxy(piperidin-2-yl)methyl)quinoline,
(2-(4-chlorophenyl)quinolin-4-yl)(pyrrolidin-2-yl)methanol,
(2-(4-ethynylphenyl)quinolin-4-yl)(piperidin-2-yl)methanol
(2-(4-chlorophenyl)quinolin-4-yl)(1-methylpiperidin-2-yl)methanol,
(R)-(2-(4-chlorophenyl)quinolin-4-yl)((S)-piperidin-2-yl)methanol,
(S)-(2-(4-chlorophenyl)quinolin-4-yl)((R)-piperidin-2-yl)methanol,
(S)-(2-(4-chlorophenyl)quinolin-4-yl)((S)-piperidin-2-yl)methanol, and
(R)-(2-(4-chlorophenyl)quinolin-4-yl)((R)-piperidin-2-yl)methanol
   or a pharmaceutically acceptable salt, solvate or prodrug thereof, for use in the treatment of glioma, and specifically glioblastoma.

Another aspect is a pharmaceutical composition comprising a therapeutically effective amount of a compound selected from
tert-butyl 4-(4-(hydroxy(piperidin-2-yl)methyl)quinolin-2-yl)benzyl(methyl)carbamate,
2-(4-chlorophenyl)-4-(methoxy(piperidin-2-yl)methyl)quinoline,
(2-(4-chlorophenyl)quinolin-4-yl)(pyrrolidin-2-yl)methanol,
(2-(4-ethynylphenyl)quinolin-4-yl)(piperidin-2-yl)methanol
(2-(4-chlorophenyl)quinolin-4-yl)(1 (S)-methylpiperidin-2-yl)methanol,
(R)-(2-(4-chlorophenyl)quinolin-4-yl)((S)-piperidin-2-yl)methanol,
(S)-(2-(4-chlorophenyl)quinolin-4-yl)((R)-piperidin-2-yl)methanol,
(S)-(2-(4-chlorophenyl)quinolin-4-yl)((S)-piperidin-2-yl)methanol, and
(R)-(2-(4-chlorophenyl)quinolin-4-yl)((R)-piperidin-2-yl)methanol or a pharmaceutically acceptable salt, solvate or prodrug thereof, and at least one pharmaceutically acceptable excipient.

Another aspect is a compound selected from
mixture of 5-(4-((R)-hydroxy((S)-piperidin-2-yl)methyl)quinolin-2-yl)-2-methylbenzonitrile and 5-(4-((S)-hydroxy((R)-piperidin-2-yl)methyl)quinolin-2-yl)-2-methylbenzonitrile,
mixture of 4-(4-((R)-hydroxy((S)-piperidin-2-yl)methyl)quinolin-2-yl)-N,N-dipropylbenzamide and 4-(4-((S)-hydroxy((R)-piperidin-2-yl)methyl)quinolin-2-yl)-N,N-dipropylbenzamide,
mixture of (R)-((S)-piperidin-2-yl)(2-(6-(trifluoromethyl)pyridin-3-yl)quinolin-4-yl)methanol and (S)-((R)-piperidin-2-yl)(2-(6-(trifluoromethyl)pyridin-3-yl)quinolin-4-yl)methanol,
mixture of (R)-((R)-piperidin-2-yl)(2-(6-(trifluoromethyl)pyridin-3-yl)quinolin-4-yl)methanol and (S)-((S)-piperidin-2-yl)(2-(6-(trifluoromethyl)pyridin-3-yl)quinolin-4-yl)methanol
   or a pharmaceutically acceptable salt, solvate or prodrug thereof, for use in therapy of cancers associated with altered Ras/Rac activity.

Another aspect is a pharmaceutical composition comprising a therapeutically effective amount of a compound selected from
mixture of 5-(4-((R)-hydroxy((S)-piperidin-2-yl)methyl)quinolin-2-yl)-2-methylbenzonitrile and 5-(4-((S)-hydroxy((R)-piperidin-2-yl)methyl)quinolin-2-yl)-2-methylbenzonitrile,
mixture of 4-(4-((R)-hydroxy((S)-piperidin-2-yl)methyl)quinolin-2-yl)-N,N-dipropylbenzamide and 4-(4-((S)-hydroxy((R)-piperidin-2-yl)methyl)quinolin-2-yl)-N,N-dipropylbenzamide,
mixture of (R)-((S)-piperidin-2-yl)(2-(4-(trifluoromethyl)phenyl)quinolin-4-yl)methanol and (S)-((R)-piperidin-2-yl)(2-(4-(trifluoromethyl)phenyl)quinolin-4-yl)methanol,
mixture of (R)-((S)-piperidin-2-yl)(2-(6-(trifluoromethyl)pyridin-3-yl)quinolin-4-yl)methanol and (S)-((R)-piperidin-2-yl)(2-(6-(trifluoromethyl)pyridin-3-yl)quinolin-4-yl)methanol,
mixture of (R)-((R)-piperidin-2-yl)(2-(4-(trifluoromethyl)phenyl)quinolin-4-yl)methanol and (S)-((S)-piperidin-2-yl)(2-(4-(trifluoromethyl)phenyl)quinolin-4-yl)methanol,
mixture of (R)-((R)-piperidin-2-yl)(2-(6-(trifluoromethyl)pyridin-3-yl)quinolin-4-yl)methanol and (S)-((S)-piperidin-2-yl)(2-(6-(trifluoromethyl)pyridin-3-yl)quinolin-4-yl)methanol
   or a pharmaceutically acceptable salt, solvate or prodrug thereof, and at least one pharmaceutically acceptable excipient.

Specifically, the invention relates to the preferred use of the R,S and/or S,R isomers of all of the aforementioned compounds for use in the treatment of cancers associated with altered Ras/Rac activity.

A further aspect of the invention relates to the use of compounds of formula (I), including stereoisomers and tautomers thereof, or a pharmaceutically acceptable salt, solvate or prodrug thereof for the treatment of glioma.

A further aspect of the invention relates to the use of compounds of formula (I), including stereoisomers and tautomers thereof, or a pharmaceutically acceptable salt, solvate or prodrug thereof for the treatment of glioblastoma.

Yet another Yet another aspect is the use of a compound of formula (I), including stereoisomers and tautomers thereof, or a pharmaceutically acceptable salt, solvate or prodrug thereof in the manufacture of a medicament for the treatment of cancers associated with altered Ras/Rac activity.

Yet another aspect is the use of a compound of formula (I), including stereoisomers and tautomers thereof, or a pharmaceutically acceptable salt, solvate or prodrug thereof in the manufacture of a medicament for the treatment of glioma.

Yet another aspect is the use of a compound of formula (I), including stereoisomers and tautomers thereof, or a pharmaceutically acceptable salt, solvate or prodrug thereof, in the manufacture of a medicament for the treatment of glioblastoma.

Yet another aspect is a method for the treatment of cancers associated with altered Ras/Rac, whereby a compound of formula (I), including stereoisomers and tautomers thereof, as defined herein above or a pharmaceutically acceptable salt, solvate or prodrug thereof is administered to a mammal, preferably a human, in need of such treatment.

Yet another aspect is a method for the treatment of glioma, whereby a compound of formula (I), including stereoisomers and tautomers thereof, as defined herein above or a pharmaceutically acceptable salt, solvate or prodrug thereof is administered to a mammal, preferably a human, in need of such treatment.

Yet another aspect is a method for the treatment of glioblastoma, whereby a compound of formula (I), including stereoisomers and tautomers thereof, as defined herein above or a pharmaceutically acceptable salt, solvate or prodrug thereof is administered to a mammal, preferably a human, in need of such treatment.

The present invention also provides (R)-[2-(4-chlorophenyl)quinolin-4-yl](2S)-piperidin-2-ylmethanol or a composition comprising (R)-[2-(4-chlorophenyl)quinolin-4-yl](2S)-piperidin-2-ylmethanol. The composition can comprise greater than 90%, greater than 95% or greater than 99% (R)-[2-(4-chlorophenyl)quinolin-4-yl](2S)-piperidin-2-ylmethanol. In some embodiments, the composition can comprise less than 1%, less than 0.5% or less than 0.1% (S)-[2-(4-chlorophenyl)quinolin-4-yl](2R)-piperidin-2-ylmethanol.

In some embodiments, the composition can comprise less than 1%, less than 0.5% or less than 0.1% (S)-[2-(4-chlorophenyl)quinolin-4-yl](2R)-piperidin-2-ylmethanol, (R)-[2-(4-chlorophenyl)quinolin-4-yl](2R)-piperidin-2-ylmethanol, and/or (S)-[2-(4-chlorophenyl)quinolin-4-yl](2S)-piperidin-2-ylmethanol.

The present invention also provides a chirally purified (R)-[2-(4-chlorophenyl)quinolin-4-yl](2S)-piperidin-2-ylmethanol comprising less than 1%, less than 0.7%, less than 0.5% or less than 0.1% (S)-[2-(4-chlorophenyl)quinolin-4-yl](2R)-piperidin-2-ylmethanol.

The present invention also provides (S)-[2-(4-chlorophenyl)quinolin-4-yl](2R)-piperidin-2-ylmethanol or a composition comprising (S)-[2-(4-chlorophenyl)quinolin-4-yl](2R)-piperidin-2-ylmethanol. The composition can comprise greater than 90%, greater than 95% or greater than 99% (S)-[2-(4-chlorophenyl)quinolin-4-yl](2R)-piperidin-2-ylmethanol. In some embodiments, the composition can comprise less than 1%, less than 0.5% or less than 0.1% (R)-[2-(4-chlorophenyl)quinolin-4-yl](2S)-piperidin-2-ylmethanol.

In some embodiments, the composition can comprise less than 1%, less than 0.5% or less than 0.1% (R)-[2-(4-chlorophenyl)quinolin-4-yl](2S)-piperidin-2-ylmethanol, (S)-[2-(4-chlorophenyl)quinolin-4-yl](2S)-piperidin-2-ylmethanol, and/or (R)-[2-(4-chlorophenyl)quinolin-4-yl](2R)-piperidin-2-ylmethanol.

The present invention also provides a chirally purified(S)-[2-(4-chlorophenyl)quinolin-4-yl](2R)-piperidin-2-ylmethanol comprising less than 1%, less than 0.7%, less than 0.5% or less than 0.1% (R)-[2-(4-chlorophenyl)quinolin-4-yl](2S)-piperidin-2-ylmethanol.

Another aspect of the present invention is (R)-[2-(4-chlorophenyl)quinolin-4-yl](2S)-piperidin-2-ylmethanol, or a pharmaceutically acceptable salt, solvate or prodrug thereof, or a composition comprising (R)-[2-(4-chlorophenyl)quinolin-4-yl](2S)-piperidin-2-ylmethanol or a pharmaceutically acceptable salt, solvate or prodrug thereof, for use in the treatment of cancers associated with altered Ras/Rac activity.

A further aspect of the invention relates to the use of (R)-[2-(4-chlorophenyl)quinolin-4-yl](2S)-piperidin-2-ylmethanol, or a pharmaceutically acceptable salt, solvate or prodrug thereof, or a composition comprising (R)-[2-(4-chlorophenyl)quinolin-4-yl](2S)-piperidin-2-ylmethanol or a pharmaceutically acceptable salt, solvate or prodrug thereof for the treatment of glioma.

A further aspect of the invention relates to the use of (R)-[2-(4-chlorophenyl)quinolin-4-yl](2S)-piperidin-2-ylmethanol, or a pharmaceutically acceptable salt, solvate or prodrug thereof, or a composition comprising (R)-[2-(4-chlorophenyl)quinolin-4-yl](2S)-piperidin-2-ylmethanol or a pharmaceutically acceptable salt, solvate or prodrug thereof for the treatment of glioblastoma.

Yet another aspect is the use of (R)-[2-(4-chlorophenyl)quinolin-4-yl](2S)-piperidin-2-ylmethanol, or a pharmaceutically acceptable salt, solvate or prodrug thereof, or a composition comprising (R)-[2-(4-chlorophenyl)quinolin-4-yl](2S)-piperidin-2-ylmethanol or a pharmaceutically acceptable salt, solvate or prodrug thereof in the manufacture of a medicament for the treatment of cancers associated with altered Ras/Rac activity.

Yet another aspect is the use of (R)-[2-(4-chlorophenyl)quinolin-4-yl](2S)-piperidin-2-ylmethanol, or a pharmaceutically acceptable salt, solvate or prodrug thereof, or a composition comprising (R)-[2-(4-chlorophenyl)quinolin-4-yl](2S)-piperidin-2-ylmethanol or a pharmaceutically acceptable salt, solvate or prodrug thereof in the manufacture of a medicament for the treatment of glioma.

Yet another aspect is the use of (R)-[2-(4-chlorophenyl)quinolin-4-yl](2S)-piperidin-2-ylmethanol, or a pharmaceutically acceptable salt, solvate or prodrug thereof, or a composition comprising (R)-[2-(4-chlorophenyl)quinolin-4-yl](2S)-piperidin-2-ylmethanol or a pharmaceutically acceptable sale, solvate or prodrug thereof in the manufacture of a medicament for the treatment of glioblastoma.

Yet another aspect is a method for the treatment of cancers associated with altered Ras/Rac, whereby (R)-[2-(4-chlorophenyl)quinolin-4-yl](2S)-piperidin-2-ylmethanol, or a pharmaceutically acceptable salt, solvate or prodrug thereof, or a composition comprising (R)-[2-(4-chlorophenyl)quinolin-4-yl](2S)-piperidin-2-ylmethanol or a pharmaceutically acceptable salt, solvate or prodrug thereof is administered to a mammal, e.g., a human, in need of such treatment.

Yet another aspect is a method for the treatment of glioma, whereby (R)-[2-(4-chlorophenyl)quinolin-4-yl](2S)-piperidin-2-ylmethanol, or a pharmaceutically acceptable salt, solvate or prodrug thereof, or a composition comprising (R)-[2-(4-chlorophenyl)quinolin-4-yl](2S)-piperidin-2-ylmethanol or a pharmaceutically acceptable salt, solvate or prodrug thereof is administered to a mammal, e.g., a human, in need of such treatment.

Yet another aspect is a method for the treatment of glioblastoma, whereby (R)-[2-(4-chlorophenyl)quinolin-4-yl](2S)-piperidin-2-ylmethanol, or a pharmaceutically acceptable salt, solvate or prodrug thereof, or a composition comprising (R)-[2-(4-chlorophenyl)quinolin-4-yl](2S)-piperidin-2-ylmethanol or a pharmaceutically acceptable salt, solvate or prodrug thereof is administered to a mammal, e.g., a human, in need of such treatment.

Another aspect of the present invention is (S)-[2-(4-chlorophenyl)quinolin-4-yl](2R)-piperidin-2-ylmethanol, or a pharmaceutically acceptable salt, solvate or prodrug thereof, or a composition comprising (S)-[2-(4-chlorophenyl)quinolin-4-yl](2R)-piperidin-2-ylmethanol or a pharmaceutically acceptable salt, solvate or prodrug thereof, for use in the treatment of cancers associated with altered Ras/Rac activity.

A further aspect of the invention relates to the use of (S)-[2-(4-chlorophenyl)quinolin-4-yl](2R)-piperidin-2-ylmethanol, or a pharmaceutically acceptable salt, solvate or prodrug thereof, or a composition comprising (S)-[2-(4-chlorophenyl)quinolin-4-yl](2R)-piperidin-2-ylmethanol or a pharmaceutically acceptable salt, solvate or prodrug thereof for the treatment of glioma.

A further aspect of the invention relates to the use of (S)-[2-(4-chlorophenyl)quinolin-4-yl](2R)-piperidin-2-ylmethanol, or a pharmaceutically acceptable salt, solvate or prodrug thereof, or a composition comprising (S)-[2-(4-chlorophenyl)quinolin-4-yl](2R)-piperidin-2-ylmethanol or a pharmaceutically acceptable salt, solvate or prodrug thereof for the treatment of glioblastoma.

Yet another aspect is the use of (S)-[2-(4-chlorophenyl)quinolin-4-yl](2R)-piperidin-2-ylmethanol, or a pharmaceutically acceptable salt, solvate or prodrug thereof, or a composition comprising (S)-[2-(4-chlorophenyl)quinolin-4-yl](2R)-piperidin-2-ylmethanol or a pharmaceutically acceptable salt, solvate or prodrug thereof in the manufacture of a medicament for the treatment of cancers associated with altered Ras/Rac activity.

Yet another aspect is the use of (S)-[2-(4-chlorophenyl)quinolin-4-yl](2R)-piperidin-2-ylmethanol, or a pharmaceutically acceptable salt, solvate or prodrug thereof, or a composition comprising (S)-[2-(4-chlorophenyl)quinolin-4-yl](2R)-piperidin-2-ylmethanol or a pharmaceutically acceptable salt, solvate or prodrug thereof in the manufacture of a medicament for the treatment of glioma.

Yet another aspect is the use of(S)-[2-(4-chlorophenyl)quinolin-4-yl](2R)-piperidin-2-ylmethanol, or a pharmaceutically acceptable salt, solvate or prodrug thereof, or a composition comprising (S)-[2-(4-chlorophenyl)quinolin-4-yl](2R)-piperidin-2-ylmethanol or a pharmaceutically acceptable sale, solvate or prodrug thereof in the manufacture of a medicament for the treatment of glioblastoma.

Yet another aspect is a method for the treatment of cancers associated with altered Ras/Rac, whereby (R)-[2-(4-chlorophenyl)quinolin-4-yl](2S)-piperidin-2-ylmethanol, or a pharmaceutically acceptable salt, solvate or prodrug thereof, or a composition comprising (S)-[2-(4-chlorophenyl)quinolin-4-yl](2R)-piperidin-2-ylmethanol or a pharmaceutically acceptable salt, solvate or prodrug thereof is administered to a mammal, e.g., a human, in need of such treatment.

Yet another aspect is a method for the treatment of glioma, whereby (S)-[2-(4-chlorophenyl)quinolin-4-yl](2R)-piperidin-2-ylmethanol, or a pharmaceutically acceptable salt, solvate or prodrug thereof, or a composition comprising (S)-[2-(4-chlorophenyl)quinolin-4-yl](2R)-piperidin-2-ylmethanol or a pharmaceutically acceptable salt, solvate or prodrug thereof is administered to a mammal, e.g., a human, in need of such treatment.

Yet another aspect is a method for the treatment of glioblastoma, whereby (S)-[2-(4-chlorophenyl)quinolin-4-yl](2R)-piperidin-2-ylmethanol, or a pharmaceutically acceptable salt, solvate or prodrug thereof, or a composition comprising (S)-[2-(4-chlorophenyl)quinolin-4-yl](2R)-piperidin-2-ylmethanol or a pharmaceutically acceptable salt, solvate or prodrug thereof is administered to a mammal, e.g., a human, in need of such treatment.

The present invention also provides a pharmaceutical composition comprising (R)-[2-(4-chlorophenyl)quinolin-4-yl](2S)-piperidin-2-ylmethanol. The pharmaceutical composition can comprise greater than 90%, greater than 95% or greater than 99% (R)-[2-(4-chlorophenyl)quinolin-4-yl](2S)-piperidin-2-ylmethanol. In some embodiments, the pharmaceutical composition can comprise less than 1%, less than 0.5% or less than 0.1% (S)-[2-(4-chlorophenyl)quinolin-4-yl](2R)-piperidin-2-ylmethanol. In some embodiments, the pharmaceutical composition can comprise less than 1%, less than 0.5% or less than 0.1% (S)-[2-(4-chlorophenyl)quinolin-4-yl](2R)-piperidin-2-ylmethanol, (R)-[2-(4-chlorophenyl)quinolin-4-yl](2R)-piperidin-2-ylmethanol, and/or (S)-[2-(4-chlorophenyl)quinolin-4-yl](2S)-piperidin-2-ylmethanol.

The present invention also provides a pharmaceutical composition comprising (S)-[2-(4-chlorophenyl)quinolin-4-yl](2R)-piperidin-2-ylmethanol. The pharmaceutical composition can comprise greater than 90%, greater than 95% or greater than 99% (S)-[2-(4-chlorophenyl)quinolin-4-yl](2R)-piperidin-2-ylmethanol. In some embodiments, the pharmaceutical composition can comprise less than 1%, less than 0.5% or less than 0.1% (R)-[2-(4-chlorophenyl)quinolin-4-yl](2S)-piperidin-2-ylmethanol. In some embodiments, the pharmaceutical composition can comprise less than 1%, less than 0.5% or less than 0.1% (R)-[2-(4-chlorophenyl)quinolin-4-yl](2S)-piperidin-2-ylmethanol, (R)-[2-(4-chlorophenyl)quinolin-4-yl](2R)-piperidin-2-ylmethanol, and/or (S)-[2-(4-chlorophenyl)quinolin-4-yl](2S)-piperidin-2-ylmethanol.

The present invention also provides a method for preparing selectively (R)-[2-(4-chlorophenyl)quinolin-4-yl](2S)-piperidin-2-ylmethanol, (S)-[2-(4-chlorophenyl)quinolin-4-yl](2R)-piperidin-2-ylmethanol, (R)-[2-(4-chlorophenyl)quinolin-4-yl](2R)-piperidin-2-ylmethanol, and/or (S)-[2-(4-chlorophenyl)quinolin-4-yl](2S)-piperidin-2-ylmethanol.

For example, tritylation of methylated (S)-L-Pipecolic acid affords the possibility to generate a chiral piperidine carbaldehyde material suitable for face-selective addition by the Grignard reagent generated from 2,4-dibromoquinoline. The single isolated R,S isomer is then subject to Suzuki coupling of the appropriate 4-chlorophenylboronic acid, which after concomitant deprotection of the trityl group yields the desired (R)-[2-(4-chlorophenyl)quinolin-4-yl](2S)-piperidin-2-ylmethanol.

For example, (R)-[2-(4-chlorophenyl)quinolin-4-yl](2S)-piperidin-2-ylmethanol is generated in several steps, by converting the (S)-L-Pipecolic acid to the corresponding ester, e.g., methyl (2S)-1-piperidine-2-carboxylate, with thionyl chloride followed by treatment with methanol, or other reagents suitable to form a chiral carboxylate. The intermediate ester is then protected with a suitable protecting group, such as a trityl group, to form a nitrogen-protected carboxylate, e.g., methyl (2S)-1-(triphenymethyl)piperidine-2-carboxylate, which is then converted to the corresponding alcohol, e.g., by reducing with a suitable reagent such as LiAlH₄. The [(2S)-1-(triphenylmethyl)piperidine-2-yl]methanol is then converted to the corresponding aldehyde by reacting with a suitable oxidizing agent, such as oxalyl chloride (e.g., Swern oxidation), the resultant (2S)-1-(triphenylmethyl)piperidine-2-carbaldehyde is then reacted with a face-selective Grignard reagent generated in situ from an appropriate reagent, such as 2,4-dibromoquinoline to yield the single R,S isomer, (R)-(2-bromoquinolin-4-yl)[(2S)-1-(triphenylmethyl)piperidin-2-yl]methanol. This bromo compound is then subjected to Suzuki coupling with the appropriate phenylboronic acid (e.g., 4-chlorophenylboronic acid) to yield (R)-[2-(4-chlorophenyl)quinolin-4-yl](2S)-1-(triphenylmethyl)piperidin-2-yl]methanol, which, after removal of the N-protecting group (e.g., trityl) produces (R)-[2-(4-chlorophenyl)quinolin-4-yl](2S)-piperidin-2-ylmethanol.

Preferably, the produced (R)-[2-(4-chlorophenyl)quinolin-4-yl](2S)-piperidin-2-ylmethanol comprises less than 1%, less than 0.7%, less than 0.5% or less than 0.1% (S)-[2-(4-chlorophenyl)quinolin-4-yl](2S)-piperidin-2-ylmethanol.

The invention may be useful for cancers with de-regulated pathways leading to increased vacuolization, such as increased Ras/Rac and/or downstream signalling pathways, observed in the majority of human cancers. Specifically, the cancers may include all types of solid tumors and hematological cancers associated with elevated levels or Ras and/or Rac overactivity, such as cancer in tissues of adrenal gland, autonomic ganglia, biliary tract, bone, breast, central nervous system, cervix, endometrium, hematopoietic/lymphoid, kidney, large intestine, liver, lung, esophagus, ovary, pancreas, prostate, salivary gland, skin, small intestine, stomach, testis, thymus, thyroid, upper aerodigstive tract, urinary tract (Ian A. Prior., Paul D Lewis, Carla Mattos (2012) “A comprehensive survey of Ras mutations in cancer.” Cancer Research 72, 2457-2467).

More specifically, the cancers for treatment with the compounds and methods described herein may include all types of gliomas regarding glioma classification, i.e. ependymomas, astrocytomas, oligodendrogliomas and mixed gliomas of all four grades (grade I-IV) and in all possible locations. Preferably the type of gliomas is astrocytomas. More preferably the astrocytomas are glioblastomas, such as GBM. The glioblastoma e.g. may be selected from proneural, classical and mesenchymal glioblastoma.

In one aspect, the compounds of the invention are for use in combinational therapy. For example, treatment of a subject with a compound of the invention may also include surgical removal of a cancer. For example, combinational therapy with a compound of the invention may also include administering radiation therapy. For example, combinational therapy with a compound of the invention may also include administration of a further anticancer agent, and/or combinations with the therapies herein described. Such combinational therapies can be concurrent, sequential or in alternation.

The invention also relates to the use the aforementioned compounds for the delivery of substances such as therapeutic DNA, gene products, cytotoxic agents, antibodies, cell penetrating peptides, nanoparticles or other agents, into cells by induced macropinocytosis.

The invention further relates to the use of a compound defined above, for the delivery of desired molecules or substances to cancer cells such as glioblastoma cells, in particular therapeutic agents. Such molecules/substances include therapeutic DNA, gene products, cytotoxic agents, antibodies, cell penetrating peptides, nanoparticles or other agents, which could kill glioma cells in vivo. Also, the delivery of imaging molecules selectively to glioma cells, such as glioblastoma cells, using the compound(s) of the invention will give the possibility to achieve cancer cell-, such as glioblastoma cell-, specific imaging.

A further aspect of the invention is a screening assay for identification of such anti-carcinogenic compounds, and a screening tool for identification of compounds active against brain tumors. Said novel screening assay is described in more detail below.

Finally, a method for selectively modulating macropinocytosis-mediated cell death in cancer cells with altered Ras/Rac activity and specifically glioma cells is an aspect of the invention.

Unless otherwise defined, all technical and scientific terms used herein have the same meaning as commonly understood by one of ordinary skill in the art to which this invention belongs. In the specification, the singular forms also include the plural unless the context clearly dictates otherwise. Although methods and materials similar or equivalent to those described herein can be used in the practice or testing of the present invention, suitable methods and materials are described below. All publications, patent applications, patents, and other references mentioned herein are incorporated by reference. The references cited herein are not admitted to be prior art to the claimed invention. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIG. 1 depicts graphs showing effect and dose-response curves of Vaquinol-1. (A, B) and (C-K) the effect on cell cycle of GSCs upon treatment with DMSO or Vacquinol-1, respectively: dose-response of Vacquinol-1 concentrations in viability assay (ATP) on different GSC density (C), 1 day GSC treatment with vacquinol-1 (D), 1 day GSC treatment with TMZ (E), 1 day mouse Glia treatment with vacquinol-1 (F), 1 day fibroblast treatment with vacquinol-1 (G), 2 days GSC treatment with vacquinol-1 (H), 3 day GSC treatment with vacquinol-1 (I), 4 days GSC treatment with vacquinol-1 (J), 4 day fibroblast treatment with vacquinol-1 (K).

FIG. 2 is a bar graph illustrating induction of a non-apoptotic death by Vacquinol-1, a caspase assay and fluorescence quantification of caspase 3 and caspase 7 after 5 mM to 30 mM Vacquinol-1 treatment of GSC from 5 min to 600 min when compared to Staurosporin (10 mM) or DMSO. Concentrations on X-axis are in micromolar.

FIG. 3 shows Western-blot analysis of GSC treated with Vacquinol-1 for 5 min to 26 h as indicated. Cell extracts were immunoblotted for phosphor-MKK4 (P-MKK4) and histone H3 trimethylation at lysine 27 (H3K27me3).

FIG. 4 shows immunohistochemical staining images of mouse brains (A, B) and corresponding statistical analysis in staple diagrams (C, D). Immunohistochemical staining with anti-human GFAP antibody on GSC xenotransplanted brains treated with DMSO (A) or Vacquinol-1 (B). Quantification of GFAP-positive (C) and necrotic area (D) is also shown.

FIG. 5 shows the four different isomers of Vacquinol-1 (S10) assigned as (R,S; S20), (S,R; S21), (S,S; S22) and (R,R; S23). Upon stereoselective synthesis of the individual isomers, an differential pharmacological activity was observed indicating that the R,S and S,R isomers showed superior in vitro activity in comparison to the R,R and S,S isomers (see also Table 4).

FIG. 6 A is a graph showing comparative systemic (plasma) exposure of racemic Vacquinol-1 (NSC13316), with enantiomerically pure Vacquinol-1 RS and Vacquinol-1 SR and FIG. 6 B is a graph showing comparative brain exposure after a single oral administration of 20 mg/kg.

FIG. 7A is a graph of the comparison of Vacquinol-1 RS (S20) and mefloquine cytotoxicity against human fibroblasts; FIG. 7B is a graph of the comparison of Vacquinol-1 RS (S20) and mefloquine cytotoxicity against glioblastoma cells (U3013).

Abbreviations used in the figures: GSC: glioma stem cells, HFS: human fibroblast, ESC: Mouse embryonic stem cells, TMZ: Temozolomide, mGlia: Mouse Glia cells, Vacq: Vacquinol-1, Sta: Staurosporin, RLUs: relative luminescence.

### DETAILED DESCRIPTION OF THE INVENTION

The foregoing and other aspects of the present invention will now be described in more detail with respect to the description and methodologies provided herein. It should be appreciated that the invention may be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the embodiments of the invention, the singular forms “a,” “an” and “the” are intended to include the plural forms as well, unless the context clearly indicates otherwise. Also, as used herein, “and/or” refers to and encompasses any and all possible combinations of one or more of the associated listed items. Furthermore, the term “about,” as used herein when referring to a measurable value such as an amount of a compound, dose, time, temperature, and the like, is meant to encompass variations of 20%, 10%, 5%, 1%, 0.5%, or even 0.1% of the specified amount. When a range is employed (e.g., a range from x to y) it is it meant that the measurable value is a range from about x to about y, or any range therein, such as about x₁ to about y₁, etc. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this invention belongs.

The present invention relates to new compounds, certain 2,4-disubstituted quinoline derivatives, to their use in therapy, as well as to a pharmaceutical composition comprising said compounds. More specifically the invention relates to certain 2,4-disubstituted quinoline derivatives or pharmaceutical compositions comprising said compounds for the treatment of cancers associated with altered Ras/Rac activity. Even more specifically, the invention relates to certain 2,4-disubstituted quinoline derivatives or pharmaceutical compositions comprising said compounds for the treatment of glioma. The invention further relates to assays for identifying such compounds. The present invention aims at providing molecules capable of selectively killing tumor cells with minimal effects on other cell types of the body.

A phenotypic screen using a library of structurally diverse small molecules with the aim to identify cellular processes in glioblastoma cells and glioblastoma stem cells (GSCs) amenable for development of targeted treatments was performed, resulting in the quinine derivative NSC13316 being the only hit molecule reliably compromising viability of glioblastoma cells and GSC, stimulating macropinocytosis-mediated cell death. Synthetic chemical expansion of NSC13316 resulted in a series of structural analogs with increased potency, which were termed Vacquinols (Table 1) due to their induction of a unique phenotypic response in glioblastoma cells and GSC. Vacquinols stimulate, in nanomolar concentrations, a non-apoptotic cell death characterized by membrane blebbing and ruffling, cell rounding, massive macropinocytic vacuole accumulation, ATP depletion and eventual disruption of the cytoplasmic membrane and cell lysis of gliomablastoma cells and GSCs of proneural, mesenchymal and classical subclasses of GBM without effects on other cell types. A genome-wide shRNA screen reveals that Vacquinols rapidly activate, and are dependent on, the MAP kinase MKK4 for vacuole induction and to excert their cytotoxic effects. In vivo xenograft models demonstrate high tolerance and GBM tumor specificity of Vacquinol-1 (Table 1, S10), which displays excellent in vivo pharmacokinetics and brain exposure following oral administration, and significantly attenuate tumor infiltration and growth in zebrafish and mouse models of human GBM.

The Vacquinols (the compound(s)) of the invention were shown to induce non-clathrin-dependent vacuolization in gliomablastoma cells. Clathrin-independent endocytosis, such as for instance macropinocytosis, results in a non-specific cellular uptake of fluid, solutes, membrane, ligands, molecules and particles in the fluid phase. This mechanism is induced by activating specific signaling pathways, which leads to alterations in plasma membrane dynamics, such as those resulting from changes in actin dynamics. This type of endocytosis is the consequence of plasma membrane ruffles that, when collapsing, results in the formation of large irregularly shaped fluid-filled endocytic vacuoles. By targeted activation of this process, fluid uptake can be massively elevated and this process is paralleled by an unselective uptake of particles.

As defined by the underlying mechanisms, clathrin-independent endocytosis segregates from other endocytic pathways. Unlike both endocytosis and phagocytosis, the clathrin-independent endocytosis is not regulated by interactions of cargo/receptor molecules, which coordinate the activity. Instead, activation of tyrosine kinase receptors, integrins, GPCRs or other cell surface receptors can lead to a selective but general elevation of actin polymerization at the cell surface, resulting in membrane ruffling that close at their distal margins to engulf extracellular fluid (Haigler et al., 1979; Mercer and Helenius, 2012; Swanson, 2008). Thus, when ruffles curve into open, crater-like cups at the cell surface membrane, ruffle closure is followed by cup closure, separating the vacuole from the plasma membrane. Hence, this mechanism is highly regulated by interactions with cell surface factors of the cell and by activation of signaling pathways driving this process. Cell type selectivity can therefore be very high. The consequence of activation of vacuolization of this type is the permeabilisation of an otherwise impermeable cell. This is exemplified by the cellular entry of many pathogens (i.e. protozoa, bacteria and virus) via this mechanism and the capture of antigens by antigen presenting cells, such as dendritic cells (Mercer J., Helenius A. (2012) Curr Opinion in Microbiology 15, 490-499; Phey, Lim; Gleeson, Pa. (2011), Immunology and Cell Biology 89, 836-843). The intracellular signaling pathway underlying this type of vacuolization involves specific proteins, such as Na+/H+ exchangers, Rho-like GTPases (for instance Rac or Cdc42), p21-activated kinase I (PAK1) and protein kinases and protein lipases. Hyperstimulation of macropinocytosis can lead to massive accumulation of cytoplasmic vacuoles and non-apoptotic death. The origin, mechanism and consequence of cytoplasmic vacuolization vary depending on the nature of the inducer as well as the cell types where vacuoles expand. Vacuoles are often cleared thus, can be reversible.

Macropinocytosis requires Ras activation. A variety of cancers are associated with mutations in rat sarcoma (HRAS, KRAS, NRAS) genes, which encode the Ras proteins, that are small GTPases with key regulatory functions for cell proliferation, growth and differentiation in a variety of cells in response to growth stimuli. Mutations resulting in constitutively active Ras can thus fuel uncontrolled cell growth, motily and proliferation. In accordance, mutations resulting in overactive Ras are found in approximately 30% of all human cancers and altered Ras/Rac activity has been reported in a majority of human cancers, including, but not limited to, pancreatic, lung, thyroid, urinary tract, lung, colorectal, salivary, prostate, intenstinal, skin, hematological/lymphoid malignancies and cervical cancer. It is believed that the effects of Vacquinols extend also to other cancer types in which overactive Ras or Ras/Rac pathway is present.

Tumor-initiating cancer cells, similar to other stem-like cells, have unique molecular features that should open up for selective targeting of cancer, for treatment of cancer, specifically cancers associated with altered Ras/Rac activity, such as gliomas, and more specifically glioblastoma (also referred to herein as glioblastoma multiforme, or GBM). The present invention relates to providing compounds capable of selectively killing tumor cells and/or cancer stem cells with minimal effects on other cell types of the body. More specifically, the invention relates to the preparation and use of 2,4-disubstituted quinoline derivatives in the treatment of cancers associated with altered Ras/Rac activity, such as, but not limited to, pancreatic, lung, thyroid, urinary tract, colorectal, salivary, prostate, intestinal, skin, hematological/lymphoid malignancies, gliomas and cervical cancer.

Further, the invention also relates to uses of a new non-clathrin-dependent vacuolization cell death mechanism selective for cancers with altered Ras/Rac activity and/or downstream signaling pathway and specifically glioma cells, in particular glioblastoma cells. The selective vacuolization may be used, e.g., for delivery of desired compounds or substances selectively to cancer cells, specifically glioma cells, in particular glioblastoma cells, or for the delivery of imaging molecules for use in selective imaging of cancer cells, specifically glioma cells, in particular glioblastoma cells. The compounds of the invention may be used to achieve this selective vacuolization, or any other suitable compound inducing said same selective vacuolization mechanism in cancer cells, specifically, glioma cells, in particular glioblastoma cells. Moreover, the invention also relates to a novel zebrafish screening assay for identifying such compounds effective in the treatment of cancer, specifically gliomas, in particular glioblastomas, and/or compounds inducing said cancer, specifically glioma cell, in particular glioblastoma cell-specific vacuolization.

Consequently, one aspect of the present invention is a compound of formula (I)

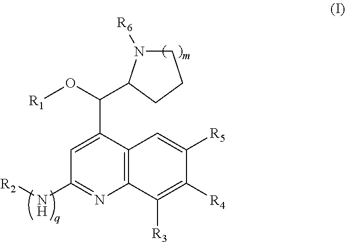

including stereoisomers and tautomers thereof, wherein

m is 1, 2 or 3;

q is 0 or 1;

R₁ is H or C1-C3 alkyl;

R₂ is selected from C1-C6 alkyl; and C3-C10 unsaturated or saturated, mono- or polycyclic carbocyclyl, and heterocyclyl or heteroaryl, each optionally substituted with one or more radicals R₇;

R₃, R₄ and R₅ are independently selected from H, halogen and C1-C6 alkyl optionally substituted with one or more halogens; or

R₃ and R₄, together with the adjacent atoms to which they are attached, form a benzene ring, and

R₅ is selected from H, halogen and C1-C6 alkyl optionally substituted with one or more halogens;

R₆ is H or C1-C3 alkyl;

each R₇ is independently selected from C1-C6 alkoxy, C1-C6 alkyl, C1-C6 alkynyl, C1-C6 alkenyl, halogen, alkylamino and NR₈C(O)OR₉;

R₈ is selected from H and C1-C3 alkyl; and

R₉ is C1-C6 alkyl, heteroaromatic or phenyl;

or a pharmaceutically acceptable salt, solvate or prodrug of the compound(s) of the formula (I), for use in the treatment of cancers associated with altered Ras/Rac activity.

For example, the compound of formula I is not mefloquine. For example, R2 is not unsubstituted pyridyl.

For example, the invention relates to a compound of formula I selected from compounds S8, S9, S14, S16, S19, S20, S21, S22, S23.

For example, the invention relates to a compound of formula I selected from compounds S24, S25, S26, S27, S28, and S29.

In some embodiments, the compound of the invention is a compound of formula I wherein m is 1 or 2.

In some embodiments, the compound of the invention is a compound of formula I wherein q is 0.

In some embodiments, the compound of the invention is a compound of formula I wherein m is 2 and q is 0.

In some embodiments, the compound of the invention is a compound of formula I wherein R2 is C6-C10 unsaturated or saturated, mono- or polycyclic carbocyclyl.

In some embodiments, the compound of the invention is a compound of formula I wherein R2 is phenyl.

In some embodiments, the compound of the invention is a compound of formula I wherein R2 is heteroaryl.

In some embodiments, the compound of the invention is a compound of formula I wherein R2 is not unsubstituted pyridyl.

In some embodiments, the invention relates to the use of a compound selected from S1, S2, S3, S4, S5, S6, S7, S8, S9, S10, S11, S12, S13, S14, S15, S16, S17, S18, S19, S20, S21, S22, S23, S24, S25, S26, S27, S28, and S29.

A further aspect of the invention relates to the use of compounds of formula (I), including stereoisomers and tautomers thereof, or a pharmaceutically acceptable salt, solvate or prodrug thereof for the treatment of glioma.

A further aspect of the invention relates to the use of compounds of formula (I), including stereoisomers and tautomers thereof, or a pharmaceutically acceptable salt, solvate or prodrug thereof for the treatment of glioblastoma.

Yet another aspect is the use of a compound of formula (I), including stereoisomers and tautomers thereof, or a pharmaceutically acceptable salt, solvate or prodrug thereof in the manufacture of a medicament for the treatment of cancers associated with altered Ras/Rac activity.

Yet another aspect is the use of a compound of formula (I), including stereoisomers and tautomers thereof, or a pharmaceutically acceptable salt, solvate or prodrug thereof in the manufacture of a medicament for the treatment of glioma.

Yet another aspect is the use of a compound of formula (I), including stereoisomers and tautomers thereof, or a pharmaceutically acceptable salt, solvate or prodrug thereof, in the manufacture of a medicament for the treatment of glioblastoma.

Yet another aspect is a method for the treatment of cancers associated with altered Ras/Rac, whereby a compound of formula (I), including stereoisomers and tautomers thereof, as defined herein above or a pharmaceutically acceptable salt, solvate or prodrug thereof is administered to a mammal, preferably a human, in need of such treatment.

Yet another aspect is a method for the treatment of glioma, whereby a compound of formula (I), including stereoisomers and tautomers thereof, as defined herein above or a pharmaceutically acceptable salt, solvate or prodrug thereof is administered to a mammal, preferably a human, in need of such treatment.

Yet another aspect is a method for the treatment of glioblastoma, whereby a compound of formula (I), including stereoisomers and tautomers thereof, as defined herein above or a pharmaceutically acceptable salt, solvate or prodrug thereof is administered to a mammal, preferably a human, in need of such treatment.

According to certain embodiments of the invention, substantially all of the composition of the invention that is used in the methods and uses described herein is the RS-enantiomer. Only a small amount of SR (or any other)-enantiomer is present. This is advantageous because the RS-enantiomer of the composition of the invention is more therapeutically effective than the SR-enantiomer or the racemic RS/SR mixture. In specific embodiments, the composition of the invention produced has less than 5% of the SR-enantiomer present by weight. In other specific embodiments, the composition of the invention produced has less than 4, 3, 2 or 1% of the SR-enantiomer present by weight. In a preferred embodiment, the composition of the invention has less than 2% of the SR-enantiomer present by weight. In a more preferred embodiment, the composition of the invention has less than 1% of the SR-enantiomer present by weight.

The present invention also provides (R)-[2-(4-chlorophenyl)quinolin-4-yl](2S)-piperidin-2-ylmethanol or a composition comprising (R)-[2-(4-chlorophenyl)quinolin-4-yl](2S)-piperidin-2-ylmethanol. The composition can comprise greater than 90%, greater than 95% or greater than 99% (R)-[2-(4-chlorophenyl)quinolin-4-yl](2S)-piperidin-2-ylmethanol. In some embodiments, the composition can comprise less than 1%, less than 0.5% or less than 0.1% (S)-[2-(4-chlorophenyl)quinolin-4-yl](2S)-piperidin-2-ylmethanol.

In some embodiments, the composition can comprise less than 1%, less than 0.5% or less than (S)-[2-(4-chlorophenyl)quinolin-4-yl](2R)-piperidin-2-ylmethanol, (R)-[2-(4-chlorophenyl)quinolin-4-yl](2R)-piperidin-2-ylmethanol, and/or (S)-[2-(4-chlorophenyl)quinolin-4-yl](2S)-piperidin-2-ylmethanol.

The present invention also provides a chirally purified (R)-[2-(4-chlorophenyl)quinolin-4-yl](2S)-piperidin-2-ylmethanol comprising less than 1%, less than 0.7%, less than 0.5% or less than 0.1% (S)-[2-(4-chlorophenyl)quinolin-4-yl](2R)-piperidin-2-ylmethanol.

The present invention also provides (S)-[2-(4-chlorophenyl)quinolin-4-yl](2R)-piperidin-2-ylmethanol or a composition comprising (S)-[2-(4-chlorophenyl)quinolin-4-yl](2R)-piperidin-2-ylmethanol. The composition can comprise greater than 90%, greater than 95% or greater than 99% (S)-[2-(4-chlorophenyl)quinolin-4-yl](2R)-piperidin-2-ylmethanol. In some embodiments, the composition can comprise less than 1%, less than 0.5% or less than 0.1% (R)-[2-(4-chlorophenyl)quinolin-4-yl](2R)-piperidin-2-ylmethanol.

The present invention also provides (S)-[2-(4-chlorophenyl)quinolin-4-yl](2R)-piperidin-2-ylmethanol or a composition comprising (S)-[2-(4-chlorophenyl)quinolin-4-yl](2R)-piperidin-2-ylmethanol. The composition can comprise greater than 90%, greater than 95% or greater than 99% (S)-[2-(4-chlorophenyl)quinolin-4-yl](2R)-piperidin-2-ylmethanol. In some embodiments, the composition can comprise less than 1%, less than 0.5% or less than 0.1% (R)-[2-(4-chlorophenyl)quinolin-4-yl](2R)-piperidin-2-ylmethanol.

In some embodiments, the composition can comprise less than 1%, less than 0.5% or less than 0.1% (R)-[2-(4-chlorophenyl)quinolin-4-yl](2S)-piperidin-2-ylmethanol, (R)-[2-(4-chlorophenyl)quinolin-4-yl](2R)-piperidin-2-ylmethanol, and/or (S)-[2-(4-chlorophenyl)quinolin-4-yl](2S)-piperidin-2-ylmethanol.

The present invention also provides a chirally purified (S)-[2-(4-chlorophenyl)quinolin-4-yl](2R)-piperidin-2-ylmethanol comprising less than 1%, less than 0.7%, less than 0.5% or less than 0.1% (R)-[2-(4-chlorophenyl)quinolin-4-yl](2S)-piperidin-2-ylmethanol.

Another aspect of the present invention is (R)-[2-(4-chlorophenyl)quinolin-4-yl](2S)-piperidin-2-ylmethanol, or a pharmaceutically acceptable salt, solvate or prodrug thereof, or a composition comprising (R)-[2-(4-chlorophenyl)quinolin-4-yl](2S)-piperidin-2-ylmethanol or a pharmaceutically acceptable salt, solvate or prodrug thereof, for use in the treatment of cancers associated with altered Ras/Rac activity.

A further aspect of the invention relates to the use of (R)-[2-(4-chlorophenyl)quinolin-4-yl](2S)-piperidin-2-ylmethanol, or a pharmaceutically acceptable salt, solvate or prodrug thereof, or a composition comprising (R)-[2-(4-chlorophenyl)quinolin-4-yl](2S)-piperidin-2-ylmethanol or a pharmaceutically acceptable salt, solvate or prodrug thereof for the treatment of glioma.

A further aspect of the invention relates to the use of (R)-[2-(4-chlorophenyl)quinolin-4-yl](2S)-piperidin-2-ylmethanol, or a pharmaceutically acceptable salt, solvate or prodrug thereof, or a composition comprising (R)-[2-(4-chlorophenyl)quinolin-4-yl](2S)-piperidin-2-ylmethanol or a pharmaceutically acceptable salt, solvate or prodrug thereof for the treatment of glioblastoma.

Yet another aspect is the use of (R)-[2-(4-chlorophenyl)quinolin-4-yl](2S)-piperidin-2-ylmethanol, or a pharmaceutically acceptable salt, solvate or prodrug thereof, or a composition comprising (R)-[2-(4-chlorophenyl)quinolin-4-yl](2S)-piperidin-2-ylmethanol or a pharmaceutically acceptable salt, solvate or prodrug thereof in the manufacture of a medicament for the treatment of cancers associated with altered Ras/Rac activity.

Yet another aspect is the use of (R)-[2-(4-chlorophenyl)quinolin-4-yl](2S)-piperidin-2-ylmethanol, or a pharmaceutically acceptable salt, solvate or prodrug thereof, or a composition comprising (R)-[2-(4-chlorophenyl)quinolin-4-yl](2S)-piperidin-2-ylmethanol or a pharmaceutically acceptable salt, solvate or prodrug thereof in the manufacture of a medicament for the treatment of glioma.

Yet another aspect is the use of (R)-[2-(4-chlorophenyl)quinolin-4-yl](2S)-piperidin-2-ylmethanol, or a pharmaceutically acceptable salt, solvate or prodrug thereof, or a composition comprising (R)-[2-(4-chlorophenyl)quinolin-4-yl](2S)-piperidin-2-ylmethanol or a pharmaceutically acceptable sale, solvate or prodrug thereof in the manufacture of a medicament for the treatment of glioblastoma.

Yet another aspect is a method for the treatment of cancers associated with altered Ras/Rac, whereby (R)-[2-(4-chlorophenyl)quinolin-4-yl](2S)-piperidin-2-ylmethanol, or a pharmaceutically acceptable salt, solvate or prodrug thereof, or a composition comprising (R)-[2-(4-chlorophenyl)quinolin-4-yl](2S)-piperidin-2-ylmethanol or a pharmaceutically acceptable salt, solvate or prodrug thereof is administered to a mammal, e.g., a human, in need of such treatment.

Yet another aspect is a method for the treatment of glioma, whereby (R)-[2-(4-chlorophenyl)quinolin-4-yl](2S)-piperidin-2-ylmethanol, or a pharmaceutically acceptable salt, solvate or prodrug thereof, or a composition comprising (R)-[2-(4-chlorophenyl)quinolin-4-yl](2S)-piperidin-2-ylmethanol or a pharmaceutically acceptable salt, solvate or prodrug thereof is administered to a mammal, e.g., a human, in need of such treatment.

Yet another aspect is a method for the treatment of glioblastoma, whereby (R)-[2-(4-chlorophenyl)quinolin-4-yl](2S)-piperidin-2-ylmethanol, or a pharmaceutically acceptable salt, solvate or prodrug thereof, or a composition comprising (R)-[2-(4-chlorophenyl)quinolin-4-yl](2S)-piperidin-2-ylmethanol or a pharmaceutically acceptable salt, solvate or prodrug thereof is administered to a mammal, e.g., a human, in need of such treatment.

Another aspect of the present invention is (S)-[2-(4-chlorophenyl)quinolin-4-yl](2R)-piperidin-2-ylmethanol, or a pharmaceutically acceptable salt, solvate or prodrug thereof, or a composition comprising (S)-[2-(4-chlorophenyl)quinolin-4-yl](2R)-piperidin-2-ylmethanol or a pharmaceutically acceptable salt, solvate or prodrug thereof, for use in the treatment of cancers associated with altered Ras/Rac activity.

A further aspect of the invention relates to the use of (S)-[2-(4-chlorophenyl)quinolin-4-yl](2R)-piperidin-2-ylmethanol, or a pharmaceutically acceptable salt, solvate or prodrug thereof, or a composition comprising (S)-[2-(4-chlorophenyl)quinolin-4-yl](2R)-piperidin-2-ylmethanol or a pharmaceutically acceptable salt, solvate or prodrug thereof for the treatment of glioma.

A further aspect of the invention relates to the use of (S)-[2-(4-chlorophenyl)quinolin-4-yl](2R)-piperidin-2-ylmethanol, or a pharmaceutically acceptable salt, solvate or prodrug thereof, or a composition comprising (S)-[2-(4-chlorophenyl)quinolin-4-yl](2R)-piperidin-2-ylmethanol or a pharmaceutically acceptable salt, solvate or prodrug thereof for the treatment of glioblastoma.

Yet another aspect is the use of (S)-[2-(4-chlorophenyl)quinolin-4-yl](2R)-piperidin-2-ylmethanol, or a pharmaceutically acceptable salt, solvate or prodrug thereof, or a composition comprising (S)-[2-(4-chlorophenyl)quinolin-4-yl](2R)-piperidin-2-ylmethanol or a pharmaceutically acceptable salt, solvate or prodrug thereof in the manufacture of a medicament for the treatment of cancers associated with altered Ras/Rac activity.

Yet another aspect is the use of (S)-[2-(4-chlorophenyl)quinolin-4-yl](2S)-piperidin-2-ylmethanol, or a pharmaceutically acceptable salt, solvate or prodrug thereof, or a composition comprising (R)-[2-(4-chlorophenyl)quinolin-4-yl](2S)-piperidin-2-ylmethanol or a pharmaceutically acceptable salt, solvate or prodrug thereof in the manufacture of a medicament for the treatment of glioma.

Yet another aspect is the use of (S)-[2-(4-chlorophenyl)quinolin-4-yl](2R)-piperidin-2-ylmethanol, or a pharmaceutically acceptable salt, solvate or prodrug thereof, or a composition comprising (S)-[2-(4-chlorophenyl)quinolin-4-yl](2R)-piperidin-2-ylmethanol or a pharmaceutically acceptable salt, solvate or prodrug thereof in the manufacture of a medicament for the treatment of glioblastoma.

Yet another aspect is a method for the treatment of cancers associated with altered Ras/Rac, whereby (S)-[2-(4-chlorophenyl)quinolin-4-yl](2R)-piperidin-2-ylmethanol, or a pharmaceutically acceptable salt, solvate or prodrug thereof, or a composition comprising (S)-[2-(4-chlorophenyl)quinolin-4-yl](2R)-piperidin-2-ylmethanol or a pharmaceutically acceptable salt, solvate or prodrug thereof is administered to a mammal, e.g., a human, in need of such treatment.

Yet another aspect is a method for the treatment of glioma, whereby (S)-[2-(4-chlorophenyl)quinolin-4-yl](2R)-piperidin-2-ylmethanol, or a pharmaceutically acceptable salt, solvate or prodrug thereof, or a composition comprising (S)-[2-(4-chlorophenyl)quinolin-4-yl](2R)-piperidin-2-ylmethanol or a pharmaceutically acceptable salt, solvate or prodrug thereof is administered to a mammal, e.g., a human, in need of such treatment.

Yet another aspect is a method for the treatment of glioblastoma, whereby (S)-[2-(4-chlorophenyl)quinolin-4-yl](2R)-piperidin-2-ylmethanol, or a pharmaceutically acceptable salt, solvate or prodrug thereof, or a composition comprising (S)-[2-(4-chlorophenyl)quinolin-4-yl](2R)-piperidin-2-ylmethanol or a pharmaceutically acceptable salt, solvate or prodrug thereof is administered to a mammal, e.g., a human, in need of such treatment.

The present invention also provides a method for preparing (R)-[2-(4-chlorophenyl)quinolin-4-yl](2S)-piperidin-2-ylmethanol and (S)-[2-(4-chlorophenyl)quinolin-4-yl](2R)-piperidin-2-ylmethanol. The synthesis can be a modification of, e.g., León (León, B., et al (2013). Organic Letters, 15(6), 1234-7).

Briefly, tritylation of methylated (S)-L-Pipecolic acid affords the possibility to generate a chiral piperidine carbaldehyde material suitable for face-selective addition by the Grignard reagent generated from 2,4-dibromoquinoline. The single isolated R,S isomer is then subject to Suzuki coupling of the appropriate 4-chlorophenylboronic acid, which after concomitant deprotection of the trityl group yields the desired (R)-[2-(4-chlorophenyl)quinolin-4-yl](2S)-piperidin-2-ylmethanol.

For example, (R)-[2-(4-chlorophenyl)quinolin-4-yl](2S)-piperidin-2-ylmethanol is generated in several steps, by converting the (S)-L-Pipecolic acid to the corresponding ester, e.g., methyl (2S)-1-piperidine-2-carboxylate, with thionyl chloride followed by treatment with methanol, or other reagents suitable to form a chiral carboxylate. The intermediate ester is then protected with a suitable protecting group, such as a trityl group, to form a nitrogen-protected carboxylate, e.g., methyl (2S)-1-(triphenymethyl)piperidine-2-carboxylate, which is then converted to the corresponding alcohol, e.g., by reducing with a suitable reagent such as LiAlH₄.

The [(2S)-1-(triphenylmethyl)piperidine-2-yl]methanol is then converted to the corresponding aldehyde by reacting with a suitable oxidizing agent, such as oxalyl chloride (e.g., Swern oxidation), the resultant (2S)-1-(triphenylmethyl)piperidine-2-carbaldehyde is then reacted with a face-selective Grignard reagent generated in situ from an appropriate reagent, such as 2,4-dibromoquinoline to yield the single R,S isomer, (R)-(2-bromoquinolin-4-yl)[(2S)-1-(triphenylmethyl)piperidin-2-yl]methanol. This bromo compound is then subjected to Suzuki coupling with the appropriate phenylboronic acid (e.g., 4-chlorophenylboronic acid) to yield (R)-[2-(4-chlorophenyl)quinolin-4-yl](2S)-1-(triphenylmethyl)piperidin-2-yl]methanol, which, after removal of the N-protecting group (e.g., trityl) produces (R)-[2-(4-chlorophenyl)quinolin-4-yl](2S)-piperidin-2-ylmethanol.

Preferably, the produced (R)-[2-(4-chlorophenyl)quinolin-4-yl](2S)-piperidin-2-ylmethanol comprises less than 1%, less than 0.7%, less than 0.5% or less than 0.1% (S)-[2-(4-chlorophenyl)quinolin-4-yl](2S)-piperidin-2-ylmethanol.

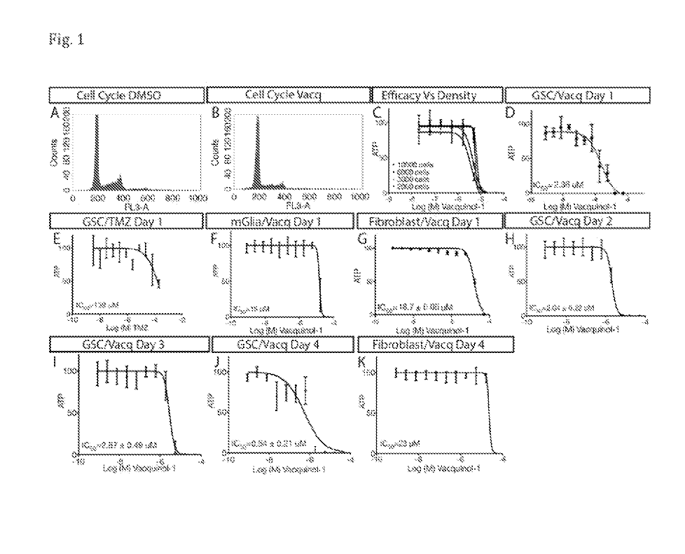

As depicted in FIG. 1, the effect of Vaquinol on cycling of GSCs (FIG. 1A, B) indicates that Vaqcuinol-1 induces a rapid and selective death of cultured GSCs, and that Vacquinol-1 is marginally affected by cell density (FIG. 1C). Vacquinol-1 has much greater efficacy than TMZ (FIG. 1E) and is selective for GSCs as mGlia and fibroblasts as well as other cell types display toxicity at higher concentrations than GSCs. Furthermore, toxicity in other cell types is independent of vacuolization, which causes death of GSCs.

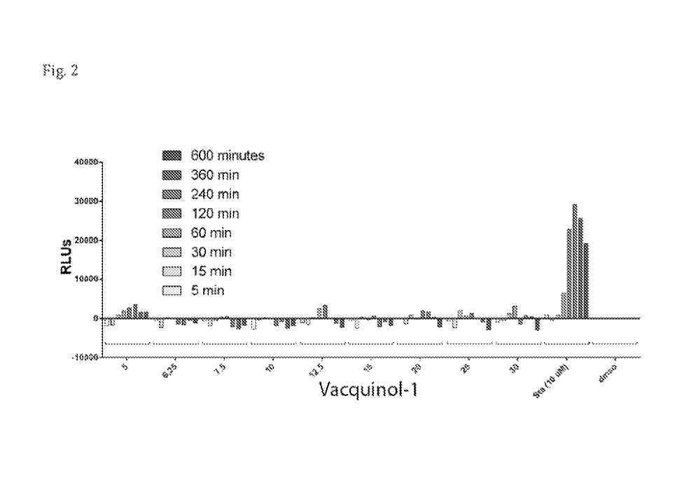

As shown in FIG. 2, illustrating the induction of non-apoptotic death by Vacquinol-1, where the absence of Caspase activation by Vacquinol-1 is evident, compared to Staurosporin, a known apoptosis inducer, which leads to rapid and marked increase of apoptotic death.

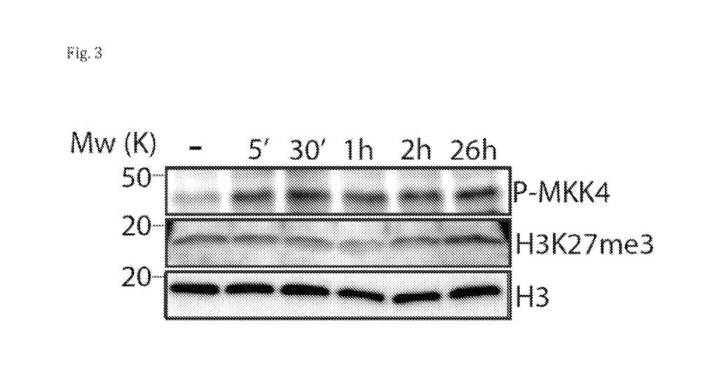

FIG. 3 shows a Western-blot analysis of GSC treated with Vacquinol-1 for 5 min to 26 hours. These data indicate a rapid increase of P-MKK4 but lack of inhibition effects of H3K27me3.

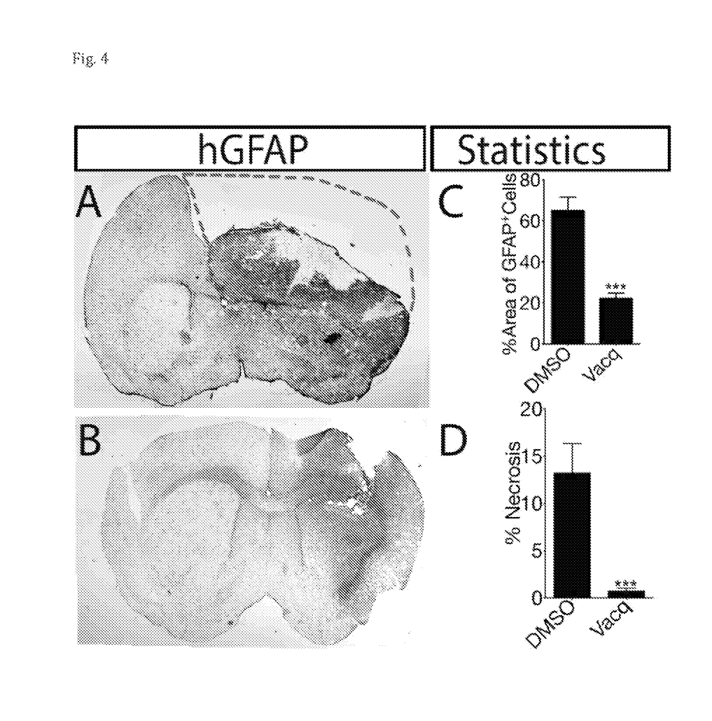

Human GSCs (100 000) were transplanted into immunodeficient mice and let to develop into a terminal stage (6 weeks) after which Vacquinol-11 (15 μM, 0.5 μL/hr) was administered by infusion into the brain for one week. Marked reduction of tumor size and attenuation of necrotic areas in Vacquinol-1 treated mice is shown in FIG. 4 A, B (unohistochemical staining images of mouse brains). Quantification via statistical analysis confirms these results (FIGS. 4 C and D, n=6/group). These data illustrate an efficient reduction of tumor development at a terminal stage in a human model of glioblastoma in mouse. Immunohistochemical staining was performed with anti-human GFAP antibody on GSC xenotransplanted brains treated with DMSO (A) or Vacquinol-1 (B). The quantification of GFAP-positive (C) and necrotic area (D).

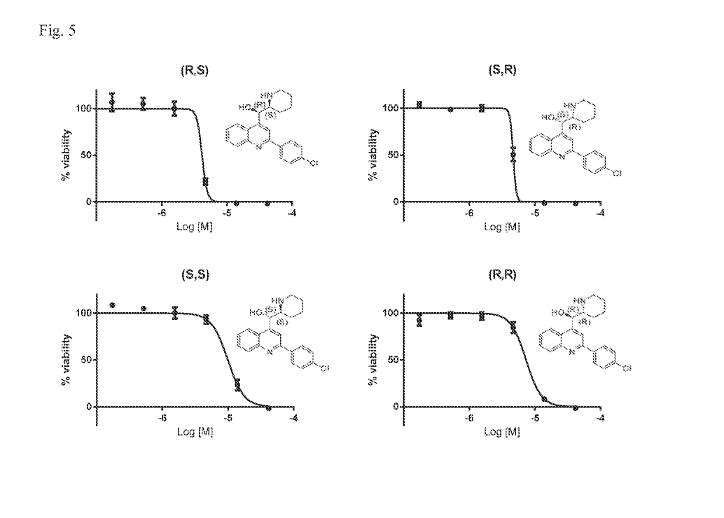

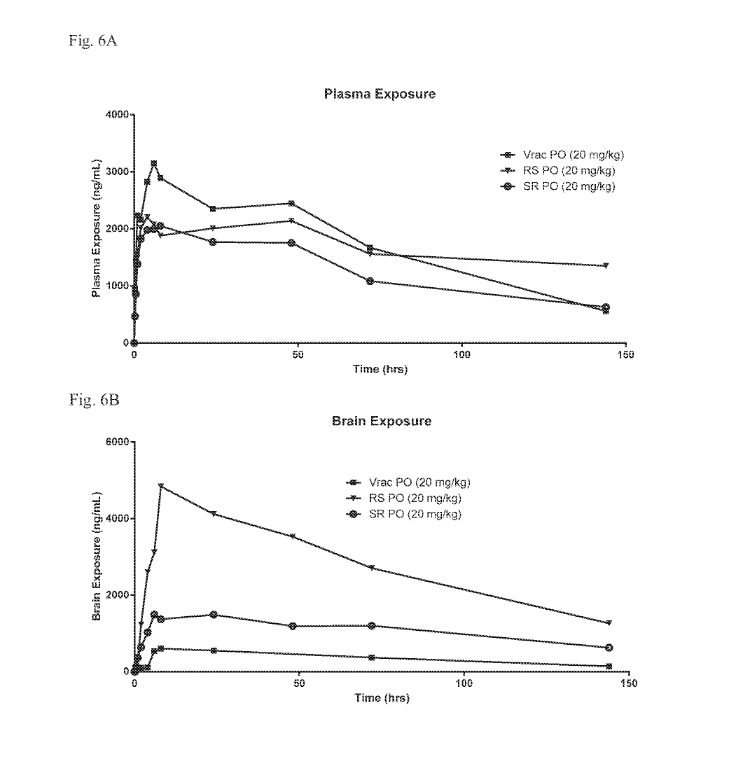

As shown in FIG. 5, upon stereoselective synthesis of the individual isomers of Vacquinol-1, a differential pharmacological activity was observed indicating that the R,S and S,R isomers showed superior in vitro activity in comparison to the R,R and S,S isomers. The pharmacokinetics of Vacquinol-1 (racemic), Vacquinol-1 RS and Vacquinol-1 SR, were determined in NMRI (SR/RS) or BALB/c (Vrac) mice following single intravenous (i.v.) or per oral (p.o) administration of 2 or 20 mg/kg Vacquinol-1, respectively. Blood and brain samples were taken from animals at the following nominal time points: 15, 30, and 60 minutes, and 2, 4, 6, 8, 24, 48, 72 and 144 hours after dosing (n=3/time-point). Bioanalytical quantification of Vacquinol-1 was analysed in plasma and brain samples by a UPLC-MS/MS. The data described herein demonstrate the superior brain exposure of Vacquinol-1RS versus the corresponding SR isomer or the previously described stereoisomeric mixture (Vacquinol-1, NSC13316), whilst minimizing systemic exposure of the compound. See, Example 11, FIG. 6. Without wishing to be bound by theory, as gliomas are pathognomonically restricted to the CNS, compounds with preferential brain exposure are more likely to be efficacious clinically with lower risks of systemic side effects.

The anti-malarial quinolinemethanol mefloquine

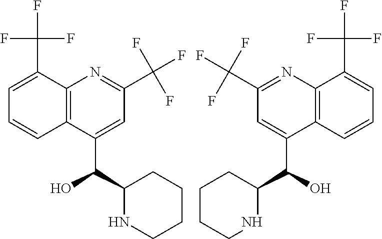

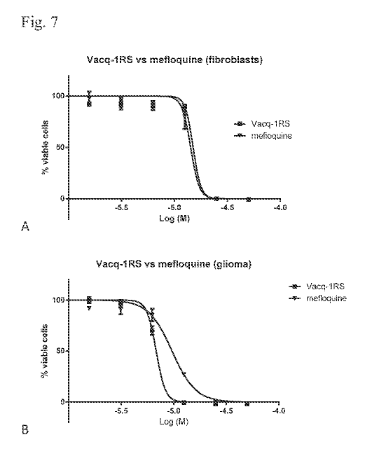

((2,8-bis(trifluoromethyl)quinolin-4-yl)(piperidin-2-yl)methanol), has been proposeed to reduce glioma cell viability by the activation of apoptosis and inhibition of autophagy (Geng, Y. et al. (2010) Neuro-Oncology, 12(5), 473-81). The study indicates that mefloquine results in roughly 50% reduction of U87 glioma cell viability at concentrations of 10 micromolar, although no proper dose-response evaluation has been made and no data is presented indicating that the compound kills all glioma cells in vitro at any concentration. Vacquinol-1, in contrast, results in complete cell culture death at comparative concentrations by an alternative mechanism, hyperactivation of macropinocytosis. In addition, the effects of Vacquinol-1 are neither caspase (apoptosis) dependent nor result in significant accumulation of autophagic vacuoles. Thus, it is unexpected that the gliomatoxic effects of mefloquine would extend to the Vacquinol series of compounds. The data in Example 12 and FIG. 7, demonstrate that while both compounds exhibit comparative cytotoxicity against fibroblasts, mefloquine kills all glioma cells only at the very highest tested concentrations. The comparative IC95 values for cell death are Vacquinol-1RS=8.9 μM and mefloquine=25.2 μM. As complete depletion of all cancer cells is a critical component of effective cancer therapy in order to avoid development of resistance and tumor recurrence, this data shows the superiority of Vacquinol-1RS is this respect.

Further, although mefloquine was shown to reduce the viability of chronic lymphocytic leukemia (CLL) and Non-Hodgkins lymphoma at high concentrations (>10 μM), no data are presented on the effects of mefloquine on neurological cancers (US2003/0216426). As cancers are highly variable, both in their pathophysiology, etiology and genetic basis, the extension of therapies from CLL and lymphomas to glioma is not intuitive or obvious.

The unexpected selective vulnerability of human patient-derived gliomablastoma cells to non-clathrin-dependent vacuolization allows for the invention of methodologies utilizing this selectivity. Due to the similarities between glioblastoma cells and other glioma cells, this mechanism may be present in all types of glioma cells, thus that the Vacquinols induce vacuolization in all types of glioma cells, as well as other cancer cells with abberant Ras/Rac activity.

Thus, a new series of structural analogs (Vacquinols) has been identified, which specifically target cancer cells without affecting other cell types. The compounds of the invention were shown to induce non-clathrin-dependent vacuolization in gliomablastoma cells resulting in cell death via a non-apoptotic mechanism. Due to the similarities between glioblastoma cells and other glioma cells, it is believed that this mechanism is present in all types of glioma cells, thus that the Vacquinols induce vacuolization in all types of glioma cells. Due to the known dependence of macropinocytosis on overactive or overexpressing Ras/Rac, it is feasible that this vulnerability extends also to other forms of cancer associated with alterations in Ras/Rac activity. These analogs open up for new treatments and therapies targeting cancer, specifically grade I-IV gliomas, including proneural, classical and mesenchymal glioblastomas. Also, a new zebrafish-based assay for identifying such compounds/analogs for the treatment of gliomas, such as glioblastoma, is disclosed as a part of the invention. See, e.g., Kitambi et al., Cell 157, 1-16, 2014, specifically incorporated herein by reference in its entirety.

Using the compounds of the invention, or other similar compounds, which induces vacuolization in glioma cells, such as glioblastoma cells, delivery of certain desired substances selectively to glioma cells could be achieved. These substances could be therapeutic substances for the treatment of disease, or they could be for example imaging molecules, such as contrast molecules, for the selective imaging of glioma cells, such as glioblastoma cells. More in detail, such a novel approach can be utilized for targeted delivery of therapeutic DNA, gene products, antibodies, cell penetrating peptides, nanoparticles or other agents, which could kill glioma cells in vivo. For example, the compound(s) of the invention may be used to improve the selectivity of otherwise unselective cytotoxic compounds, such as Temozolomide. Therefore, the selective process of vacuolization, leads to the delivery of experimental or established therapeutic agents in a tumor-targeted fashion to reduce tumor size or kill tumor cells or for visualization.

The usability of the invention is exemplified below, wherein, e.g, a range of small macromolecules can be targeted to cells by this clathrin-independent vacuolization: Compounds described herein may contribute to the efficiency of delivering cell-penetrating peptides. Kaplan, I M; Wadia, J S; Dowdy, S F. “Cationic TAT peptide transduction domain enters cells by macropinocytosis.” J Control Release (2005), 102, 247-253; Jones A T, “Macropinocytosis: searching for an endocytic identity and role in the uptake of cell penetrating peptides.” J Cell. Mol. Med (2007), 11, 670-684).

Further, compounds described herein may mediate uptake of intact proteins, including prion protein. Magzoub, M; Sandgren S; Lundberg, P; Wittrup A, et al. “N-terminal peptides from unprocessed prion proteins enter cells by macropinocytosis” Biochem Biophys, Res Commun (2006), 348, 379-385., Noguchi H, Bonner-Weir, S; Wei, F Y, et al. “BETA2/NeuroD protein can be transduced into cells due to an arginine- and lysine-rich sequence.” Diabetes 2005, 54, 2859-2866. Greenwood, K P; Daly, N L; Brown, D L; Stow J L; et al. “The cyclic cystine knot miniprotein MCoTI-II is internalized into cells by macropinocytosis” Int J Biochem Cell Biol (2007), 39, 2252-2264. Khelef, N; Gounon, P; Guiso, N; “Internalization of Bordetella pertussis adenylate cyclase-haemolysin into endocytic vesicles contributes to macrophage cytotoxicity.” Cell Microbiol (2001), 3, 721-730. Poussin, C; Foti, M; Carpentier, J L; Pugin, J. “CD14-dependent endotoxin internalization via a macropinocytic pathway.” J Biol. Chem. (1998), 273, 20285-20291.

Compounds described herein may mediate update of DNA to the cells. Wittrup A, Sandgren S, Lilja J, Bratt, Gustavsson, N. et al. “Identification of proteins released by mammalian cells that mediate DNA internalization through proteoglycan-dependent macropinocytosis.” J Biol. Chem (2007), 282, 27897-27904.

Compounds described herein may target intracellular uptake of the small molecule Lucifer Yellow and high molecular weight dextran. Zandgren, K J; Wilkinson, J; Miranda-Saksena, M; et. al. “A differential role for macropinocytosis in mediating entry of the two forms of vaccinia virus into dendritic cells.” PLoS Pathog. (2010), 6(4), e1000866. Commisso, C; Davidson, S M; Kamphorst, J J: Grabocka, E. et al. “Macropinocytosis of protein is an amino acid supply route in Ras-transformed cells.” Nature (2013), 497, 633-637.

Compounds described herein may also target uptake of engineered nanoparticles and virus-like particles. Schmidt S M, Moran K A, Slosar J L. Et. al. “Uptake of calcium phosphate nanoshells by osteoblasts and their effect on growth and differentiation.” J Biomed Mater Res A (2008), 87, 418-428. Buonaguro, L; Tornesello, M L; Tagliamonte, M; Gallo, R C; et. al. “Baculovirus-derived human immunodeficiency virus type 1 virus-like particles activate dendritic cells and induce ex vivo T-cell responses.” J Virol (2006), 80, 9134-9143.

Compounds described herein may also be useful with magnetic resonance imaging (MRI), computed tomography, X-ray and positron emission tomography (PET) and other imaging methods which can be improved upon the targeted binding or uptake of contrasting molecules. The unselective uptake process of non-clathrin dependent endocytosis, such as macropinocytosis (Kerr, M C; Teasdale, R D; “Defining macropinocytosis.” Traffic (2009), 10, 364-371), opens for targeted delivery based on cellular selectivity of induced vacuolization, such as described in this invention.

Thus, this represents a key mechanism for delivery of a range of small to large macromolecules to the cell cytoplasm from the extracellular environment. Therefore, the modulation of non-clathrin dependent vacuolization by targeting extracellular or intracellular components in the pathway selectively in glioma cells, such as glioblastoma cells, can lead to targeted strategies to deliver therapeutic agents ranging from small to large molecules and can be used for the targeted visualization of glioma tissue and cells in vivo.

The novel screening tool used for identification of compounds active against brain tumors is a further aspect of the invention. The novel assay of the invention allows for rapid evaluation of such compounds in an in vivo setup, whereby features such as the acute/chronic toxicity effect of the compounds on zebrafish and transplanted cells, transplanted cell proliferation and migration of cells into brain parenchyma, compounds penetrance into the zebrafish tissue may all be evaluated in parallel. These features make the xenograft model of the present invention a powerful tool allowing for a reduction of the number of compounds for subsequent evaluation in rodent models. The zebrafish screening assay is carried out according to the following:

Zebrafish embryos at 1 cell stage (zygote) are injected with MITFa morpholino (Lister, J A, et. al. “Nacre encodes a zebrafish microphthalmia-related protein that regulates neural-crest-derived pigment cell fate.” Development. (1999), 126(17): 3757-67) to prevent pigmentation. Pigmention can be prevented to allow for easy visualization of any phenotype of developing embryo. This can be achieved either by injection at 1 cell stage embryos with a MITFa morpholino or by the addition of 0.003% Phenyl thio urea (PTU) (0.003% 1-phenyl-2-thiourea in 1 L Tank water=60 g/ml final concentration) to the embryo. Embryos are allowed to grow for two days in the incubator after which they are collected and anesthetized using Tricaine and embedded in agarose (low melt) in a petri plate.

Tricaine (3-amino benzoic acid ethyl ester also called ethyl 3-aminobenzoate) comes in a powdered form from Sigma (Cat.#A-5040). It is also available as Finquel (Part No. C-FINQ-UE) from Argent Chemical Laboratories, Inc. Make tricaine solution for anesthetizing fish by combining the following in a glass bottle with a screw cap: 400 mg tricaine powder, 97.9 ml DD water, ˜2.1 ml 1 M Tris (pH 9). Adjust pH to ˜7. Store this solution in the freezer (buy the smallest amount possible because tricaine gets old). To use tricaine as an anesthetic combine the following in a 250 ml beaker: 4.2 ml tricaine solution and about 100 ml clean tank water. Following embedding, the agarose is allowed to solidfy and 10 ml of fresh Tricaine is added to the petri plate. The petriplate is placed under a microscope and the microinjection needle is loaded with glioma cells and the pressure of the microinjector calibrated so that each injection releases around 20-50 nl of fluid with approximately 3000 cells. The cells are injected into the brain ventricle manually, then the embryos are observed under the microscope and wrongly injected embryos are removed. The rest of the injected embryos are taken in a new plate and the tricaine treated tank water is removed and replaced with normal tank water, and the animals are allowed to recover for 3-4 hours. After 3-4 hours the animals are visually inspected to check they are swimming, then animals are distributed into a multiwell plate (3 embryos/96well plate (300 μl volume per well), 6 embryos/6 well plate (1 ml volume per well)). Drugs are then added to the plate at required concentration. The drug treated tank water is exchanged every day and the effect on the fish is monitored manually. Around 500 embryos can be injected with glioma stem cells, or glioma cells, in 3-4 hours. Accordingly, many new drug candidates can be evaluated for the treatment of glioblastoma or glioma by this fast and efficient new screening method.

A zebrafish screening assay, as described herein, has been used to identify compounds effective in the selective treatment of gliomas, especially intractable glioblastomas, and one aspect of the invention is the use of these compounds in therapy of such cancers. A new vacuolization mechanism selective for glioma cells has also been determined, which may be used for additionally susceptible forms of cancer and for selective delivery of desired compounds/molecules for use in e.g. therapy or imaging methods (e.g., cargo compounds).

For the purpose of the present invention, the term “alkyl”, either alone or as part of a radical, includes straight or branched chain alkyl of the general formula CₙH₂ₙ₊₁.

The term “Cm-Cn alkyl”, wherein m and n are both integers and m>n, refers to alkyl having from m to n carbon atoms. For example, C1-C6 alkyl includes methyl, ethyl, n-propyl and isopropyl.

For the purpose of the present invention, unless otherwise specified or apparent from the context, the term “halogen” refers to F, Cl, Br or I; preferably F, Cl and Br; in particular F and Cl.

The term “alkoxy” refers to a radical of the formula —OR, wherein R is an alkyl moiety as defined herein.

The term “alkylamino” refers to a radical of the formula —RNHR¹R², wherein R, R¹, R² is an alkyl moiety as defined herein.

The term “carbocyclyl” refers to a cyclic moiety containing only carbon (C, CH or CH₂) in the ring.

The term “heteroaryl” refers to a cyclic moiety containing carbon and one or more atoms selected from N, O, or S in the ring.

The term “polycyclic” refers to e.g. fused or bridged rings.

An unsaturated cyclic moiety may be either aromatic or non-aromatic and containing one or several double or triple bonds in the ring.

“Optional” or “optionally” means that the subsequently described event or circumstance may but need not occur, and that the description includes instances where the event or circumstance occurs and instances in which it does not.

Any chiral center in a compound of the invention having a specified configuration is indicated as R or S using the well-known Cahn-Ingold-Prelog priority rules. Also, in any structural formula a chiral center having a specified configuration, (i.e. R or S) may be indicated using

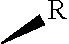

to indicate that the bond to R is directed out of the paper and towards the reader, and

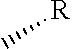

to indicate that the bond to R is directed out of the paper and away from the reader.

As used herein, a “compound” refers to the compound itself, including stereoisomers and tautomers thereof, and its pharmaceutically acceptable salts, solvates, hydrates, complexes, esters, prodrugs and/or salts of prodrugs, unless otherwise specified within the specific text for that compound. Except, when otherwise indicated, e.g. by indication of (R) or (S) configuration at a given location, all stereoisomers of the compounds of the instant invention are contemplated, either in admixture or in pure or substantially pure form. Consequently, compounds of the invention may exist in enantiomeric or racemic or diastereomeric forms or as mixtures thereof. The processes for preparation can utilize racemates or enantiomers as starting materials. When racemic and diastereomeric products are prepared, they can be separated by conventional methods, which for example are chromatographic or fractional crystallization.

The term “solvate” refers to a complex of variable stoichiometry formed e.g. by a compound of formula (I) and a solvent. The solvent is a pharmaceutically acceptable solvent, such as water, which should not interfere with the biological activity of the solute.

Some compounds of the present invention can exist in a tautomeric form which are also intended to be encompassed within the scope of the present invention. “Tautomers” refers to compounds whose structures differ markedly in arrangement of atoms, but which exist in easy and rapid equilibrium. It is to be understood that the compounds of the invention may be depicted as different tautomers. It should also be understood that when compounds have tautomeric forms, all tautomeric forms are intended to be within the scope of the invention, and the naming of the compounds does not exclude any tautomeric form.

The compounds, salts and prodrugs of the present invention can exist in several tautomeric forms, and such tautomeric forms are included within the scope of the present invention. Tautomers exist as mixtures of a tautomeric set in solution. In solid form, usually one tautomer predominates. Even though one tautomer may be described, the present invention includes all tautomers of the present compounds

As used herein, the term “salt” such as a pharmaceutically acceptable salt and can include acid addition salts including hydrochlorides, hydrobromides, phosphates, sulphates, hydrogen sulphates, alkylsulphonates, arylsulphonates, acetates, benzoates, citrates, maleates, fumarates, succinates, lactates, and tartrates; alkali metal cations such as Na⁺, K⁺, Li⁺, alkali earth metal salts such as Mg²⁺ or Ca²⁺, or organic amine salts.

By “pharmaceutically acceptable salt” it is meant those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharmaceutical Sciences, 1977, 66:1-19. The salts can be prepared in situ during the final isolation and purification of the compounds of the invention, or separately by reacting the free base function with a suitable organic acid. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxyethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

Pharmaceutically acceptable salts include acid addition salts formed with inorganic acids, e.g. hydrochloric acid, hydrobromic acid, sulfuric acid, nitric acid, phosphoric acid; or formed with organic acids, e.g. acetic acid, benzenesulfonic acid, benzoic acid, camphorsulfonic acid, citric acid, ethanesulfonic acid, fumaric acid, glucoheptonic acid, gluconic acid, glutamic acid, glycolic acid, hydroxynaphtoic acid, 2-hydroxyethanesulfonic acid, lactic acid, maleic acid, malic acid, malonic acid, mandelic acid, methanesulfonic acid, muconic acid, 2-naphthalenesulfonic acid, propionic acid, salicylic acid, succinic acid, tartaric acid, p-toluenesulfonic acid, trimethylacetic acid; or salts formed when an acidic proton present in the parent compound either is replaced by a metal ion, e.g., an alkali metal ion, an alkaline earth ion, or an aluminum ion; or coordinates with an organic or inorganic base. Acceptable organic bases include e.g. diethanolamine, ethanolamine, N-methylglucamine, triethanolamine, and tromethamine. Acceptable inorganic bases include e.g. aluminum hydroxide, calcium hydroxide, potassium hydroxide, sodium carbonate and sodium hydroxide.

For the purpose of the present invention “pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

Also provided herein is a pharmaceutical composition comprising a therapeutically effective amount of a compound of formula (I), or a pharmaceutically acceptable salt, solvate or prodrug thereof, in admixture with at least one pharmaceutically acceptable excipient, e.g. an adjuvant, diluent or carrier.

The term “effective amount” refers to an amount of a compound that confers a therapeutic effect on the treated patient. The effect may be objective (i.e. measurable by some test or marker) or subjective (i.e. the subject gives an indication of or feels an effect).

Pharmaceutically acceptable excipients for use in formulating a compound according to the invention as described and claimed herein, are for example, vehicles, adjuvants, carriers or diluents, which are well-known to those skilled in the art. Pharmaceutical excipients useful in formulating a compound as herein claimed and disclosed are found in e.g. Remington: The Science and Practice of Pharmacy, 19th ed., Mack Printing Company, Easton, Pa. (1995).

As used herein, the term “metabolite” means a product of metabolism of a compound of the present invention, or a pharmaceutically acceptable salt, polymorph or solvate thereof, that exhibits a similar activity in vivo to said compound of the present invention.

As used herein, the term “mixing” means combining, blending, stirring, shaking, swirling or agitating. The term “stirring” means mixing, shaking, agitating, or swirling. The term “agitating” means mixing, shaking, stirring, or swirling.

The term “prodrug” is intended to include any compounds which are converted by metabolic or hydrolytic processes within the body of a subject to an active agent that has a formula within the scope of the present invention. Conventional procedures for the selection and preparation of suitable prodrugs are described, for example, in Prodrugs, Sloane, K. B., Ed.; Marcel Dekker: New York, 1992, incorporated by reference herein in its entirety. The compounds of the present invention can also be prepared as prodrugs, for example pharmaceutically acceptable prodrugs. The terms “pro-drug” and “prodrug” are used interchangeably herein and refer to any compound which releases an active parent drug in vivo. Since prodrugs are known to enhance numerous desirable qualities of pharmaceuticals (e.g., solubility, bioavailability, manufacturing, etc.) the compounds of the present invention can be delivered in prodrug form. Thus, the present invention is intended to cover prodrugs of the presently claimed compounds, methods of delivering the same and compositions containing the same. The term “prodrug” includes a compound of the present invention covalently linked to one or more pro-moieties, such as an amino acid moiety or other water-solubilizing moiety. A compound of the present invention may be released from the pro-moiety via hydrolytic, oxidative, and/or enzymatic release mechanisms. In an embodiment, a prodrug composition of the present invention exhibits the added benefit of increased aqueous solubility, improved stability, and improved pharmacokinetic profiles. The pro-moiety may be selected to obtain desired prodrug characteristics. For example, the pro-moiety, e.g., an amino acid moiety or other water solubilizing moiety such as phosphate may be selected based on solubility, stability, bioavailability, and/or in vivo delivery or uptake. The term “prodrug” is also intended to include any covalently bonded carriers that release an active parent drug of the present invention in vivo when such prodrug is administered to a subject. Prodrugs in the present invention are prepared by modifying functional groups present in the compound in such a way that the modifications are cleaved, either in routine manipulation or in vivo, to the parent compound. Prodrugs include compounds of the present invention wherein a hydroxy, amino, sulfhydryl, carboxy, or carbonyl group is bonded to any group that, may be cleaved in vivo to form a free hydroxyl, free amino, free sulfhydryl, free carboxy or free carbonyl group, respectively.

Examples of prodrugs include, but are not limited to, esters (e.g., acetate, dialkylaminoacetates, formates, phosphates, sulfates, and benzoate derivatives) and carbamates (e.g., N,N-dimethylaminocarbonyl) of hydroxy functional groups, esters groups (e.g. ethyl esters, morpholinoethanol esters) of carboxyl functional groups, N-acyl derivatives (e.g. N-acetyl) N-Mannich bases, Schiff bases and enaminones of amino functional groups, oximes, acetals, ketals and enol esters of ketone and aldehyde functional groups in compounds of Formula I, and the like, See Bundegaard, H. “Design of Prodrugs” p 1-92, Elesevier, New York-Oxford (1985).

As Vacquinols intrinsically contain 2 chiral centers, the compounds evaluated in Table 1 exist as 4 stereoisomers, comprising the (R,S), (S,R), (R,R) and (S,S) isomers. Upon chiral separation of these individual stereoisomers of Vacquinol-1 (Table 1, S10) and assignment of absolute stereochemistry using X-ray crystal diffraction and NMR analysis, the (R,S) and (S,R) isomers (Table 1, S20 and S21, respectively) were found to exhibit superior activity to the (S,S) and (R,R) (Table 1, S22 and S23, respectively) isomers (FIG. 5).

Compounds of the invention may be prepared according to the synthetic routes disclosed herein, or applying synthetic methods known from literature.

In a compound of formula (I),

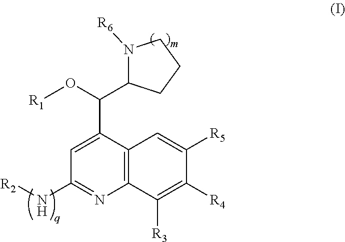

as defined herein above, m is 1 or 2, and q is 0 or 1.

In some embodiments, q is 0, i.e. the compound of the invention may be represented by formula (Ia)

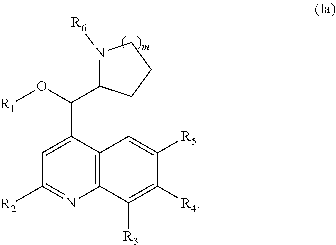

In other embodiments, q is 1, i.e. the compound of the invention may be represented by formula (Ib)

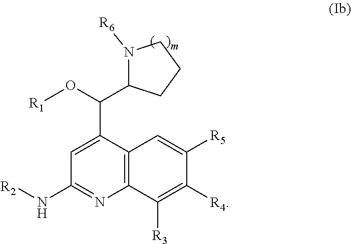

In some embodiments, m is 1, i.e. the compound of the invention may be represented by formula (Ic)

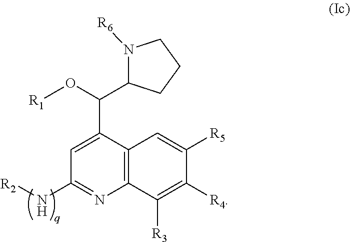

In other embodiments, m is 2, i.e. the compound of the invention may be represented by formula (Id)

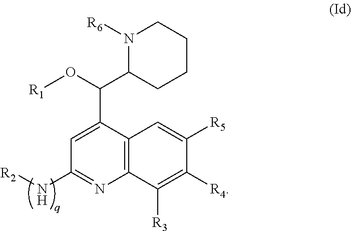

In some particular embodiments, q is 0 and m is 2.

In a compound of formula (I), R₁ is H or C1-C3 alkyl. In some embodiments, R₁ is H or methyl.

In some embodiments, R₁ is C1-C3 alkyl, e.g. R₁ is methyl.

In other embodiments, R₁ is H, i.e. the compound of the invention may be represented by formula (Ie)

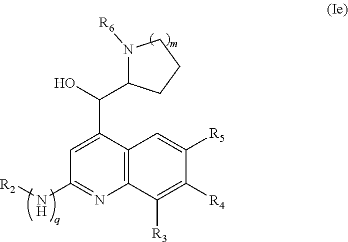

In one preferred embodiment, —OR₁ is a suitable prodrug ester, phosphate ester, sulfonate ester, hydrate, acetal, hemiacetal or any other hydrolysable or enzymatically hydrolysable group, which is cleaved intracellularly.

For example, R₁ may be C1-C6 alkyl-C(O)—, e.g. acetyl, propionyl, or butyryl; or R₁ may be benzoyl, or any other moiety forming a suitable carboxylic ester; or a corresponding phosphate ester, or sulfonate ester.

In some other particular embodiments, q is 0, m is 2 and R₁ is H.

In a compound of formula (I), R₂ is selected from C1-C6 alkyl, and C3-C10 unsaturated or saturated, mono- or polycyclic carbocyclyl, optionally substituted with one or more radicals R₇.

In some embodiments, R₂ is selected from C1-C6 alkyl, C3-C10 saturated, mono- or polycyclic carbocyclyl, optionally substituted with one or more radicals R₇; and phenyl, optionally substituted with one or more radicals R₇.

When R₂ is C3-C10 unsaturated or saturated, mono- or polycyclic carbocyclyl, optionally substituted with one or more radicals R₇, said cyclyl e.g. may be C6-C10 unsaturated or saturated, mono- or polycyclic carbocyclyl, such as C6-C10 bridged or non-bridged cycloalkyl, e.g. cyclohexyl and octahydro-1H-2,5-methanoindenyl; or phenyl.

In some other embodiments, R₂ is C3-C10 unsaturated or saturated, mono- or polycyclic carbocyclyl, optionally substituted with one or more radicals R₇, e.g. R₂ is C6-C10 unsaturated or saturated, mono- or polycyclic carbocyclyl, e.g. C6-C10 non-bridged or bridged cycloalkyl, such as cyclohexyl and octahydro-1H-2,5-methanoindenyl; or phenyl.

In some embodiments, R₂ is C3-C10 saturated, mono- or polycyclic carbocyclyl, optionally substituted with one or more radicals R₇, e.g. R₂ is C6-C10 saturated, mono- or polycyclic carbocyclyl, e.g. C6-C10 non-bridged or bridged cycloalkyl, such as cyclohexyl and octahydro-1H-2,5-methanoindenyl; or phenyl.

In some embodiments, R₂ is phenyl, optionally substituted with one or more radicals R₇, e.g. 1, 2 or 3 radicals R₇, i.e. the compound of the invention may be represented by formula (If)

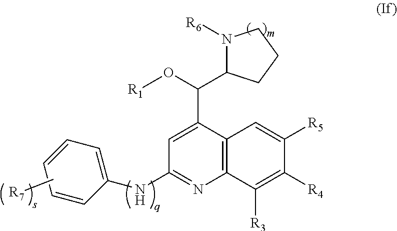

wherein s is an integer of from 0 to 5, or from 0 to 4, or from 0 to 3, or from 0 to 2, e.g. s is 0 or 1. In some embodiments, s is 0. In some embodiments, s is 1. In some embodiments, s is 2.

In some embodiments of a compound of formula (Ih), s is at least 1 and at least one radical R₇ is in para position. In some embodiments of a compound of formula (Ih), s is 1 and R₇ is in para position.

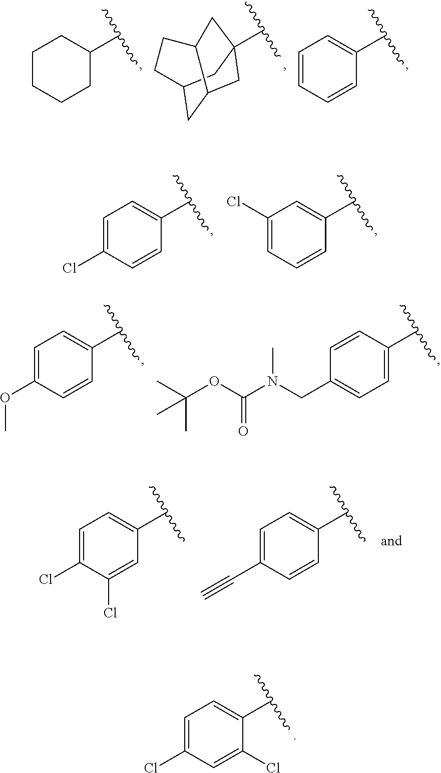

In a compound of formula (I), R₃, R₄ and R₅ are independently selected from H, halogen, such as F and Cl, and C1-C6 alkyl, e.g. C1-C3 alkyl, such as methyl, optionally substituted with one or more halogens; or R₃ and R₄, together with the adjacent atoms to which they are attached, form a benzene ring, and R₅ is selected from H, halogen, e.g. F and Cl, and C1-C6 alkyl, e.g. C1-C3 alkyl.

In some embodiments, R₃, R₄ and R₅ are independently selected from H, halogen, such as F and Cl, and C1-C6 alkyl, e.g. C1-C3 alkyl, such as methyl.

In some embodiments, R₃, R₄ and R₅ are independently selected from H and halogen, e.g. from H, F and Cl, or H and Cl.

In some other embodiments, R₃, R₄ and R₅ are independently selected from H, and C1-C6 alkyl, e.g. C1-C3 alkyl, such as methyl.

In still other embodiments, R₃, R₄ and R₅ are independently selected from H, and C1-C6 alkyl, e.g. C1-C3 alkyl, such as methyl.

In still other embodiments, R₃ and R₄, together with the adjacent atoms to which they are attached, form a benzene ring, and R₅ is selected from H, halogen, e.g. F and Cl, and C1-C6 alkyl, e.g. C1-C3 alkyl; e.g. R₃ and R₄, together with the adjacent atoms to which they are attached, form a benzene ring, and R₅ is H.

In some embodiments, R₃ is as defined herein above, but is different from H. For example, R₃ is different from H, and R₄ and R₅ are both H.

In some embodiments, R₄ is as defined herein above, but is different from H. For example, R₄ is different from H, and R₃ and R₅ are both H.

In some embodiments, R₅ is as defined herein above, but is different from H. For example, R₅ is different from H, and R₃ and R₄ are both H.

In some embodiments, both R₃ and R₅ are different from H. For example, R₃ and R₅ are as defined herein above, but are different from H, and R₄ is H.

In some other embodiments, R₃, R₄ and R₅ are all H.

In formula (I), the moiety R₆ is H or a C1-C3 alkyl, e.g. methyl. In some embodiments, R₆ is H.

As noted herein above, when R₂ is C3-C10 unsaturated or saturated, mono- or polycyclic carbocyclyl, said cyclyl may be substituted with one or more radicals R₇. Each such radical R₇ is independently selected from C1-C6 alkoxy, e.g. C1-C3 alkoxy, such as methoxy; and halogen, e.g. F and Cl, in particular Cl; and NR₈C(O)OR₉.

In some other embodiments, at least one R₇ is halogen, e.g. F or Cl, in particular Cl.

When R₇ is NR₈C(O)OR₉, R₈ is selected from H and C1-C3 alkyl, in particular H; and R₉ is C1-C6 alkyl. In some embodiments, R₈ is H, and R₉ is C3-C6 alkyl, e.g. tert-butyl.

From the above, it appears that the compound of formula (I) may vary with respect to various features. Such features relate to the integers q and m, and the identity of R₁, R₂, R₃, R₄, R₅ and R₆. It is contemplated that, unless otherwise indicated or clearly apparent from the context, the different features of the compound of formula (I) may be independently and freely combined to give rise to a multitude embodiments within the scope of the invention, which embodiments are all covered by formula (I).

Examples of compounds of the present invention for use in the treatment of cancers associated with altered Ras/Rac activity, specifically gliomas, such as glioblastoma, are:
(2-phenylbenzo[h]quinolin-4-yl)(piperidin-2-yl)methanol,
(6,8-dichloro-2-((2R,3aS,5R)-octahydro-1H-2,5-methanoinden-2-yl)quinolin-4-yl)(piperidin-2-yl)methanol,
(2-((4-chlorophenyl)amino)-6-methylquinolin-4-yl)(piperidin-2-yl)methanol,
(8-chloro-2-(4-chlorophenyl)quinolin-4-yl)(piperidin-2-yl)methanol,
(6,8-dichloro-2-phenylquinolin-4-yl)(piperidin-2-yl)methanol,
(2-(3-chlorophenyl)quinolin-4-yl)(piperidin-2-yl)methanol,
(2-(3,4-dichlorophenyl)quinolin-4-yl)(piperidin-2-yl)methanol,
tert-butyl 4-(4-(hydroxy(piperidin-2-yl)methyl)quinolin-2-yl)benzyl(methyl)carbamate,
(2-(4-chlorophenyl)quinolin-4-yl)(piperidin-2-yl)methanol,
(7-chloro-2-phenylquinolin-4-yl)(piperidin-2-yl)methanol,
(2-(2,4-dichlorophenyl)quinolin-4-yl)(piperidin-2-yl)methanol,
(6-chloro-2-phenylquinolin-4-yl)(piperidin-2-yl)methanol,
(2-(4-ethynylphenyl)quinolin-4-yl)(piperidin-2-yl)methanol
2-(4-chlorophenyl)-4-(methoxy(piperidin-2-yl)methyl)quinoline,
(2-(4-methoxyphenyl)quinolin-4-yl)(piperidin-2-yl)methanol,
(2-(4-chlorophenyl)quinolin-4-yl)(pyrrolidin-2-yl)methanol,
(6,8-dichloro-2-(trifluoromethyl)quinolin-4-yl)(piperidin-2-yl)methanol,
(2-cyclohexylquinolin-4-yl)(piperidin-2-yl)methanol,
(2-(4-chlorophenyl)quinolin-4-yl)(1-methylpiperidin-2-yl)methanol,
(R)-(2-(4-chlorophenyl)quinolin-4-yl)((S)-piperidin-2-yl)methanol,
(S)-(2-(4-chlorophenyl)quinolin-4-yl)((R)-piperidin-2-yl)methanol,
(S)-(2-(4-chlorophenyl)quinolin-4-yl)((S)-piperidin-2-yl)methanol, and
(R)-(2-(4-chlorophenyl)quinolin-4-yl)((R)-piperidin-2-yl)methanol
   or a pharmaceutically acceptable salt, solvate or prodrug thereof.

Examples of compounds of the present invention include, mixture of 5-(4-((R)-hydroxy((S)-piperidin-2-yl)methyl)quinolin-2-yl)-2-methylbenzonitrile and 5-(4-((S)-hydroxy((R)-piperidin-2-yl)methyl)quinolin-2-yl)-2-methylbenzonitrile,
mixture of 4-(4-((R)-hydroxy((S)-piperidin-2-yl)methyl)quinolin-2-yl)-N,N-dipropylbenzamide and 4-(4-((S)-hydroxy((R)-piperidin-2-yl)methyl)quinolin-2-yl)-N,N-dipropylbenzamide,
mixture of (R)-((S)-piperidin-2-yl)(2-(6-(trifluoromethyl)pyridin-3-yl)quinolin-4-yl)methanol and (S)-((R)-piperidin-2-yl)(2-(6-(trifluoromethyl)pyridin-3-yl)quinolin-4-yl)methanol,
mixture of (R)-((R)-piperidin-2-yl)(2-(6-(trifluoromethyl)pyridin-3-yl)quinolin-4-yl)methanol and (S)-((S)-piperidin-2-yl)(2-(6-(trifluoromethyl)pyridin-3-yl)quinolin-4-yl)methanol

Examples of compounds of the present invention include, mixture of 5-(4-((R)-hydroxy((S)-piperidin-2-yl)methyl)quinolin-2-yl)-2-methylbenzonitrile and 5-(4-((S)-hydroxy((R)-piperidin-2-yl)methyl)quinolin-2-yl)-2-methylbenzonitrile,
mixture of 4-(4-((R)-hydroxy((S)-piperidin-2-yl)methyl)quinolin-2-yl)-N,N-dipropylbenzamide and 4-(4-((S)-hydroxy((R)-piperidin-2-yl)methyl)quinolin-2-yl)-N,N-dipropylbenzamide,
mixture of (R)-((S)-piperidin-2-yl)(2-(4-(trifluoromethyl)phenyl)quinolin-4-yl)methanol and (S)-((R)-piperidin-2-yl)(2-(4-(trifluoromethyl)phenyl)quinolin-4-yl)methanol,
mixture of (R)-((S)-piperidin-2-yl)(2-(6-(trifluoromethyl)pyridin-3-yl)quinolin-4-yl)methanol and (S)-((R)-piperidin-2-yl)(2-(6-(trifluoromethyl)pyridin-3-yl)quinolin-4-yl)methanol,
mixture of (R)-((R)-piperidin-2-yl)(2-(4-(trifluoromethyl)phenyl)quinolin-4-yl)methanol and (S)-((S)-piperidin-2-yl)(2-(4-(trifluoromethyl)phenyl)quinolin-4-yl)methanol,
mixture of (R)-((R)-piperidin-2-yl)(2-(6-(trifluoromethyl)pyridin-3-yl)quinolin-4-yl)methanol and (S)-((S)-piperidin-2-yl)(2-(6-(trifluoromethyl)pyridin-3-yl)quinolin-4-yl)methanol

Further comprised within the scope of the present invention are stereoisomers and tautomers of the compounds of the present invention.

A preferred embodiment of the invention is the use of the (R,S) and (S,R) racemate isomers of the aforementioned compounds.

More preferred is the use of the (R,S) or (S,R) single enantiomers of the aforementioned compounds.

In particular is the use of the (R,S) or (S,R) single isomers of (2-(4-chlorophenyl)quinolin-4-yl)(piperidin-2-yl)methanol, i.e., selected from the following compounds:
(R)-(2-(4-chlorophenyl)quinolin-4-yl)((S)-piperidin-2-yl)methanol,
(S)-(2-(4-chlorophenyl)quinolin-4-yl)((R)-piperidin-2-yl)methanol.

The compounds of the invention can be administered by any suitable means, for example, orally, such as in the form of tablets, pills, dragees, aqueous or oily suspensions or solutions, elixirs, syrups, capsules, granules or powders; sublingually; buccally; parenterally, such as by e.g. subcutaneous, intravenous, intramuscular, or intrasternal injection or infusion techniques (e.g., as sterile injectable aqueous or non-aqueous solutions or suspensions). For parenteral administration, a parenterally acceptable aqueous or oily suspension, emulsion or solution is employed, which is pyrogen free and has requisite pH, isotonicity, osmolality and stability. Those skilled in the art are well able to prepare suitable formulations and numerous methods are described in the literature. A brief review of methods of drug delivery is also found in the scientific literature [eg. Langer, Science 249:1527-1533 (1990)].

Other examples of possible methods of administering the compounds of the invention are nasal administration including administration to the nasal membranes, such as by inhalation spray; or rectally such as in the form of suppositories; in dosage unit formulations containing non-toxic, pharmaceutically acceptable vehicles or diluents.

Preferably, the compounds of the present invention are parenterally administered in a way optimized for delivery to the brain of the treated subject. In one embodiment, the compounds are formulated for intraperitoneal administration. In one preferred embodiment, the compounds are formulated for intracerebroventricular administration.

The present compounds can also be administered in a form suitable for immediate release or extended release. Immediate release or extended release can be achieved by the use of suitable pharmaceutical compositions comprising the present compounds, or, particularly in the case of extended release, by the use of devices such as subcutaneous implants or osmotic pumps. The compounds of the invention can also be administered liposomally. The precise nature of the carrier or other material will depend on the route of administration and those skilled in the art are well able to prepare suitable solutions and numerous methods are described in the literature. Exemplary compositions for oral administration include suspensions which can contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which can contain, for example, microcrystalline cellulose, dicalcium phosphate, starch, magnesium stearate and/or lactose and/or other excipients, binders, extenders, disintegrants, diluents and lubricants such as those known in the art. The compounds of the invention can also be delivered through the oral cavity by sublingual and/or buccal administration. Molded tablets, compressed tablets or freeze-dried tablets are exemplary forms, which may be used. Exemplary compositions include those formulating the present compound(s) with fast dissolving diluents such as mannitol, lactose, sucrose and/or cyclodextrins. Also included in such formulations may be high molecular weight excipients such as celluloses (avicel) or polyethylene glycols (PEG). Such formulations can also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g. Carbopol 934). Lubricants, glidants, flavors, coloring agents and stabilizers may also be added for ease of fabrication and use.

Exemplary compositions for nasal aerosol or inhalation administration include solutions in saline, which can contain, for example, benzyl alcohol or other suitable preservatives, absorption promoters to enhance bioavailability, and/or other solubilizing or dispersing agents such as those known in the art.

Exemplary compositions for parenteral administration include injectable solutions, emulsions or suspensions which can contain, for example, suitable non-toxic, parenterally acceptable diluents or solvents, such as mannitol, 1,3-butanediol, water, Ringer's solution, an isotonic sodium chloride solution, oil or other suitable dispersing or wetting and suspending agents, including synthetic mono- or diglycerides, and fatty acids, including oleic acid, or Cremaphor.

Exemplary compositions for rectal administration include suppositories, which can contain, for example, a suitable non-irritating excipient, such as cocoa butter, synthetic glyceride esters or polyethylene glycols, which are solid at ordinary temperatures, but liquify and/or dissolve in the rectal cavity to release the drug.

The dose administered to a mammal, particularly a human, in the context of the present invention should be sufficient to effect a therapeutic response in the mammal over a reasonable time frame. One skilled in the art will recognize that dosage will depend upon a variety of factors including the potency of the specific compound, the age, condition and body weight of the patient, the extent of the condition being treated, recommendations of the treating physician, and the therapeutics or combination of therapeutics selected for administration, as well as the stage and severity of the disease. The dose will also be determined by the route (administration form), timing and frequency of administration. Oral dosages of the present invention, when used for the indicated effects, will range between about 0.01 mg per kg of body weight per day (mg/kg/day) to about 100 mg/kg/day, preferably 0.01 mg per kg of body weight per day (mg/kg/day) to 20 mg/kg/day, and most preferably 0.1 to 10 mg/kg/day, for adult humans. For oral administration, the compositions are preferably provided in the form of tablets or other forms of presentation provided in discrete units containing 0.5 to 1000 milligrams of the active ingredient for the symptomatic adjustment of the dosage to the patient to be treated, for example 0.5, 1.0, 2.5, 5.0, 10.0, 15.0, 25.0, 50.0, 100, 200, 400, 500, 600 and 800 mg.

Parenterally, especially intracerebroventricularly or intraperitoneally, the most preferred doses will range from about 0.001 to about 10 mg/kg/hour during a constant rate infusion. Advantageously, compounds of the present invention may be administered in single doses, e.g. once daily or more seldom, or in a total daily dosage administered in divided doses of two, three or four times daily.

Compounds of the present invention may also be used or administered in combination with at least one second therapeutic agent useful in the treatment of gliomas, such as glioblastoma. The therapeutic agents may be in the same formulation or in separate formulations for administration simultaneously or sequentially. Compounds of the present invention may also be used in a combinational therapy or administered in combination with additional therapies, such as surgery and/or irradiation and/or other therapeutic strategies, including chemotherapies.

As used herein, a “compound” refers to the compound of formula (I) itself and its pharmaceutically acceptable salts, hydrates, complexes, esters, prodrugs and/or salts of prodrugs, unless otherwise specified within the specific claims for that compound. In one preferred embodiment, R1 is a suitable prodrug ester, phosphate ester, sulfonate ester, hydrate, acetal, hemiacetal or any other hydrolysable or enzymatically hydrolysable group, which is cleaved intracellularly.

For the purpose of the present invention, the term “cancer associated with an altered Ras/Rac activity” should be understood to include all types of cancer associated with mutations in, or abbarent activity of Ras and/or Rac, such as cancer in tissues of adrenal gland, autonomic ganglia, biliary tract, bone, breast, central nervous system, cervix, endometrium, hematopoietic/lymphoid, kidney, large intestine, liver, lung, esophagus, ovary, pancreas, prostate, salivary gland, skin, small intestine, stomach, testis, thymus, thyroid, upper aerodigstive tract, urinary tract [Ian A. Prior., Paul D Lewis, Carla Mattos (2012) A comprehensive survey of Ras mutations in cancer. Cancer Research 72, 2457-2467].

For the purpose of the present invention, the term “glioma” should be understood to include all types of gliomas, i.e. ependymomas, astrocytomas, oligodendrogliomas and mixed gliomas, all grades of glioma, grade I-IV glioma tumors, and in all locations, supratentorial, infratentorial and pontine. “Glioblastoma” should be understood as synonymous with glioblastoma multiform (GBM) or grade IV astrocytoma.

The term “endocytosis” refers to an energy-using process by which cells absorb molecules (such as proteins) by engulfing them. Endocytosis includes clathrin-mediated endocytosis. Examples of non-clathrin dependent endocytosis include for example: Caveola, macropinocytosis and phagocytosis. The invention relates particularly to non-clathrin dependent endocytosis of types independent from Caveola, such as macropinocytosis.

The term “vacuolization” refers to membrane-bound organelles, which are present in all animal cells. Vacuoles are essentially enclosed compartments filled with water containing inorganic and organic molecules including enzymes in solution, though in certain cases they may contain solids, which have been engulfed. Vacuoles can be formed intracellularly by the fusion of multiple membrane vesicles to form large vesicles or from endocytosis at the cytoplasmic membrane. Vacuoles have no basic shape or size; its structure varies according to the needs of the cell.

The term “cancer stem cells” refers to cancer/tumor cells that can form new tumors in animal models or in a patient, and is used as a synonym to tumor initiating/inducing cells. Regarding glioblastoma, said cancer cells are denoted glioblastoma cancer stem cells.

The term “treatment” as used throughout the specification and claims encompasses preventive therapy, palliative therapy or curative therapy. Thus, the term “treating” (or treatment) encompasses not only treating (or treatment of) a patient to relieve the patient of the signs and symptoms of the disease or condition, or to ameliorate the condition of the patient suffering from the disease or disorder, but also prophylactically treating an asymptomatic patient to prevent the onset or progression of the disease or condition. In one embodiment, the treatment is to relieve the patient of the signs and symptoms of the disease or condition, or to ameliorate the condition of the patient suffering from the disease or disorder or to prevent progression of the disease or condition.

As used herein, “treating,” “treatment” or “treat” describes the management and care of a patient for the purpose of combating a disease, condition, or disorder and includes the administration of a compound of the present invention, to alleviate the symptoms or complications of a disease, condition or disorder, or to eliminate the disease, condition or disorder. The term “treat” can also include treatment of a cell in vitro or an animal model.

The term “patient(s)” include mammalian (including human) patient(s) (or “subject(s)”). As used herein, a “subject” is interchangeable with a “subject in need thereof”, both of which refer to a subject having a disorder in which viral infection plays a part, or a subject having an increased risk of developing cancer relative to the population at large. A “subject” includes a mammal. The mammal can be e.g., a human or appropriate non-human mammal, such as primate, mouse, rat, dog, cat, cow, horse, goat, camel, sheep or a pig. In one embodiment, the mammal is a human.

An aspect of the invention is a combination product comprising:

(A) a compound of the invention, as hereinbefore defined; and

(B) a second therapeutic agent useful in the treatment of glioblastoma, wherein each of compound (A) of the present invention, and the second therapeutic agent (B), is formulated in admixture with a pharmaceutically acceptable excipient. Such a combination product provides for the administration of a compound of the invention in conjunction with a second therapeutic agent, and may thus be presented either as a separate formulation, wherein at least one such formulation comprises a compound of the invention, and at least one comprises the second therapeutic agent, or may be presented (i.e. formulated) as a combined preparation (i.e. presented as a single formulation including a compound of the invention and the other therapeutic agent).

An aspect of the invention is a pharmaceutical formulation comprising a compound of the invention, as hereinbefore defined, and a second therapeutic agent, together with a pharmaceutically acceptable excipient, such as an adjuvant, diluent or carrier.

Yet another aspect of the invention is a kit of parts comprising:

(a) a pharmaceutical formulation comprising a compound of the invention, as hereinbefore defined, in admixture with a pharmaceutically acceptable excipient, such as an adjuvant, diluent or carrier; and

(b) a pharmaceutical formulation comprising a second therapeutic agent in admixture with a pharmaceutically acceptable excipient, such as an adjuvant, diluent or carrier;

wherein each component (a) and (b) are provided in a form suitable for administration in conjunction with the other.

The compound of the invention, as defined above, can be used for the selective delivery of desired compounds, substances and/or molecules to glioma cells in vivo or in vitro. These desired substances/compounds/molecules may be therapeutic compounds e.g. for selective killing of glioma cells, or imaging molecules, such as contrast molecules, for selective imaging of glioma cells. The therapeutic compounds may be cytotoxic compounds, therapeutic DNA, antibodies, gene products, nanoparticles or other agents having the ability to kill glioma cells in vivo.

One aspect of the invention is thus use of the compound defined above (I) for the glioma cell selective delivery of desired compounds, substances or molecules such as be cytotoxic compounds, therapeutic DNA, antibodies, gene products, nanoparticles or nanoparticles or other agents having the ability to kill glioma cells in vivo.

A further aspect of the invention is use of the selective delivery defined above, for the treatment of gliomas, such as glioblastoma.

Another aspect is use of the compound defined above (I), for the glioma cell selective delivery of imaging molecules, such as contrast molecules or contrast agents, for the imaging of glioma cells.

A further aspect of the invention is a zebrafish screening assay for evaluating the ability of a test compound for treating brain cancer comprising the steps:
a) preventing pigmentation of zebrafish embryos;
b) incubating the embryos for two days post fertilization (2 dpf) in a container;
c) anesthetizing the zebrafish
d) injecting unlabelled or dye labelled or transgene expressing brain cancer cells or cells, into the brain ventricle of the embryos;
e) allowing the zebrafish to recover from the anesthetizing;
f) distributing live swimming zebrafish into a container with multiple chambers;
g) adding test compounds to at least one container chambers;
h) monitoring the zebrafish over time to establish the efficacy of the test compound by determining increase or decrease of cells in the zebrafish brain.

For example, the brain cancer is glioma.

For example, the cancer cells are unlabelled or dye labelled (such as cell tracker) or transgene expressing (such as GFP/RFP or liciferase or doxycycline/tetracycline or tamoxifen inducible constructs) cancer cells or cells from primary tumors of brain tumor glioma cells, such as glioblastoma cells.

For example, the anesthetizing is accomplished with Tricaine.

For example, pigmentation is prevented by injecting embryos at 1 cell stage with a substance, such as morpholinos that block development of pigmentation of embryos e.g. morpholino against MITFa mRNA, or by exposing the embryos to Phenyl thio urea.

For example many test compounds are assayed at one time, but adding one of the many test compounds to a chamber containing zebrafish embbryos in a container.

A further aspect of the invention is a zebrafish screening assay for evaluating the therapeutic potential/efficacy of a compound for treating glioma, such as glioblastoma, comprising the steps:
i) prevent pigmentation of zebrafish embryos by
   i) injecting embryos at 1 cell stage with a substance, such as morpholinos that block development of pigmentation of embryos e.g. morpholino against MITFa mRNA,
      and/or
ii) adding Phenyl thio urea (PTU) to the tank water of an incubator to be used for incubating the embryos
j) put the zebrafish embryos in an incubator tank and allow the embryos to grow for two days post fertilization (2 dpf) in the incubator
k) collect the zebrafish, e.g. in a petri plate or similar container, and anesthetize them by using e.g. Tricaine embedded in agarose (low melt) in a petri plate or similar
l) inject unlabelled or dye labelled (such as cell tracker) or transgene expressing (such as GFP/RFP or liciferase or doxycycline/tetracycline or tamoxifen inducible constructs) cancer cells or cells from primary tumors of brain tumor glioma cells, such as glioblastoma cells, into the brain ventricle of the embryos
m) optionally remove wrongly injected embryos
n) replace the anesthetic containing tank water, such as tricaine treated tank water, with normal tank water in the petriplate or container
o) allow the zebrafish to recover, e.g. for about 3-4 hours
p) distribute live swimming zebrafish into a multiwell plate or similar container
q) add drugs to the wells or containers at required concentrations
r) exchange tank water in the wells or containers regularly, such as daily, with water containing said same drug concentration
s) monitor the zebrafish over time to establish the efficacy of the drug evaluated in the treatment of glioma by determining increase or decrease of glioma (glioblastoma) cells in the zebrafish brain, e.g. by monitoring the zebrafishes visually.

In some embodiments, a conjugate is a compound described herein connected to or in contact with a cargo compound.

In some embodiments, a conjugate is a compound of formula (I) connected to or in contact with a cargo compound.

In some embodiments, a conjugate is a compound selected from Table 1 connected to or in contact with a cargo compound.

The formulas of the compounds referred to herein as S1 to S29 are shown herein below, in Table 1.

TABLE 1

| Ref. | Structural formula | Formula name |
|---|---|---|
| S1* | 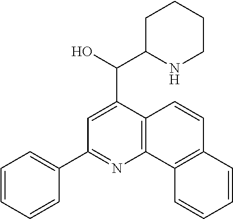 [SMILES: OC(c1cc(-c2ccccc2)nc2c1ccc1ccccc12)C1CCCCN1] | (2-phenylbenzo[h]quinolin-4- yl)(piperidin-2-yl)methanol (NSC13480) |
| S2* | 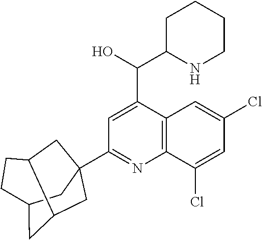 [SMILES: OC(c1cc(C23CC4CCC(C2)C(C4)C3)nc2c(Cl)cc(Cl)cc12)C1CCCCN1] | (6,8-dichloro-2-((2R,3aS,5R)- octahydro-1H-2,5-methanoinden- 2-yl)quinolin-4-yl)(piperidin-2- yl)methanol (NSC305787) |
| S3* | 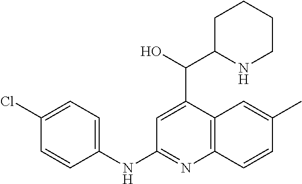 [SMILES: Cc1ccc2nc(Nc3ccc(Cl)cc3)cc(C(O)C3CCCCN3)c2c1] | (2-((4-chlorophenyl)amino)-6- methylquinolin-4-yl)(piperidin-2- yl)methanol (NSC157571) |
| S4* | 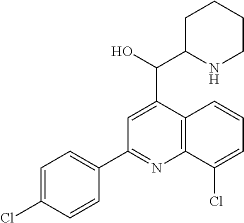 [SMILES: OC(c1cc(-c2ccc(Cl)cc2)nc2c(Cl)cccc12)C1CCCCN1] | (8-chloro-2-(4-chlorophenyl)quinolin-4- yl)(piperidin-2-yl)methanol (NSC4377) |
| S5* | 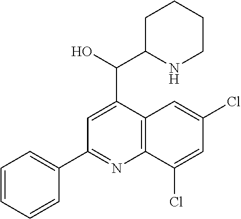 [SMILES: OC(c1cc(-c2ccccc2)nc2c(Cl)cc(Cl)cc12)C1CCCCN1] | (6,8-dichloro-2-phenylquinolin-4- yl)(piperidin-2-yl)methanol (NSC305758) |
| S6* | 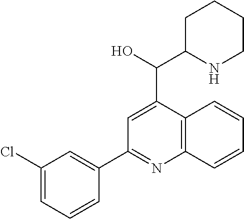 [SMILES: OC(c1cc(-c2cccc(Cl)c2)nc2ccccc12)C1CCCCN1] | (2-(3-chlorophenyl)quinolin-4-yl) (piperidin-2-yl)methanol (NSC14224) |
| S7* | 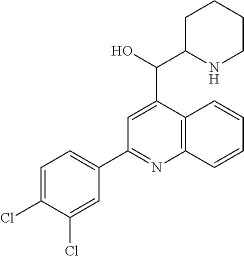 [SMILES: OC(c1cc(-c2ccc(Cl)c(Cl)c2)nc2ccccc12)C1CCCCN1] | (2-(3,4-dichlorophenyl)quinolin-4- yl)(piperidin-2-yl)methanol (NSC2450) |
| S8 | 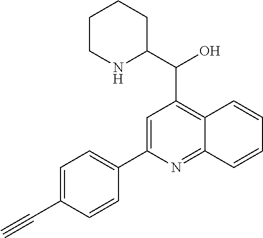 [SMILES: C#Cc1ccc(-c2cc(C(O)C3CCCCN3)c3ccccc3n2)cc1] | (2-(4-ethynylphenyl)quinolin-4-yl) (piperidin-2-yl)methanol |
| S9 | 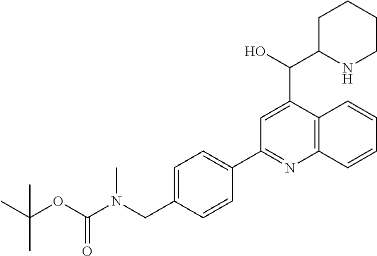 [SMILES: CN(Cc1ccc(-c2cc(C(O)C3CCCCN3)c3ccccc3n2)cc1)C(=O)OC(C)(C)C] | tert-butyl 4-(4-(hydroxy(piperidin-2- yl)methyl)quinolin-2- yl)benzyl(methyl)carbamate. |
| S10* | 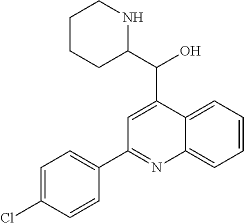 [SMILES: OC(c1cc(-c2ccc(Cl)cc2)nc2ccccc12)C1CCCCN1] | (2-(4-chlorophenyl)quinolin-4-yl)- (piperidin-2-yl)methanol (NSC13316, Vacquinol-1) |
| S11* | 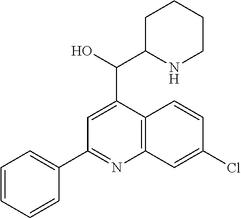 [SMILES: OC(c1cc(-c2ccccc2)nc2cc(Cl)ccc12)C1CCCCN1] | (7-chloro-2-phenylquinolin-4-yl) (piperidin-2-yl)methanol (NSC16001) |
| S12* | 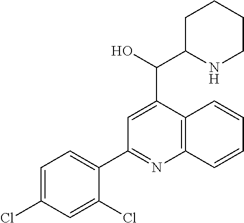 [SMILES: OC(c1cc(-c2ccc(Cl)cc2Cl)nc2ccccc12)C1CCCCN1] | (2-(2,4-dichlorophenyl)quinolin-4-yl)- (piperidin-2-yl)methanol (NSC23924) |
| S13* | 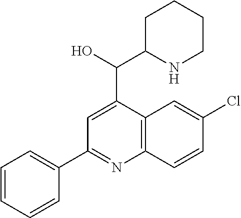 [SMILES: OC(c1cc(-c2ccccc2)nc2ccc(Cl)cc12)C1CCCCN1] | (6-chloro-2-phenylquinolin-4-yl) (piperidin-2-yl)methanol (NSC13097) |
| S14 | 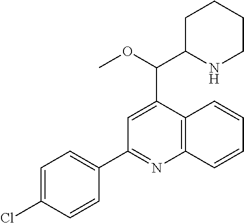 [SMILES: COC(c1cc(-c2ccc(Cl)cc2)nc2ccccc12)C1CCCCN1] | 2-(4-chlorophenyl)-4- (methoxy(piperidin-2- yl)methyl)quinoline |
| S15* | 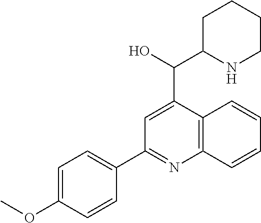 [SMILES: COc1ccc(-c2cc(C(O)C3CCCCN3)c3ccccc3n2)cc1] | (2-(4-methoxyphenyl)quinolin-4-yl)- (piperidin-2-yl)methanol (NSC23925) |
| S16 | 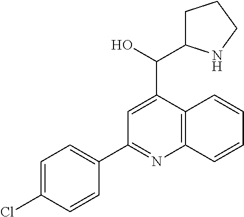 [SMILES: OC(c1cc(-c2ccc(Cl)cc2)nc2ccccc12)C1CCCN1] | (2-(4-chlorophenyl)quinolin-4-yl)- (pyrrolidin-2-yl)methanol |
| S17* | 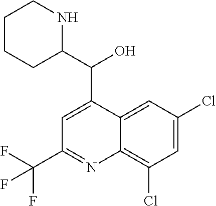 [SMILES: OC(c1cc(C(F)(F)F)nc2c(Cl)cc(Cl)cc12)C1CCCCN1] | (6,8-dichloro-2-(trifluoromethyl) quinolin-4-yl)(piperidin-2- yl)methanol (NSC322661) |
| S18* | 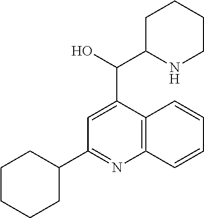 [SMILES: OC(c1cc(C2CCCCC2)nc2ccccc12)C1CCCCN1] | (2-cyclohexylquinolin-4-yl)(piperidin- 2-yl)-methanol (NSC13466) |
| S19 | 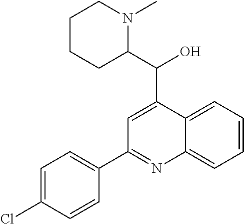 [SMILES: CN1CCCCC1C(O)c1cc(-c2ccc(Cl)cc2)nc2ccccc12] | (2-(4-chlorophenyl)quinolin-4-yl)(1- methyl-piperidin-2-yl)methanol |
| S20 | 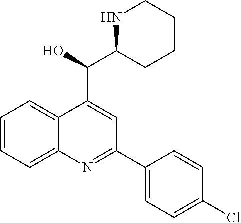 [SMILES: O[C@H](c1cc(-c2ccc(Cl)cc2)nc2ccccc12)[C@@H]1CCCCN1] | (R)-(2-(4-chlorophenyl)quinolin-4- yl)((S)-piperidin-2-yl)methanol |
| S21 | 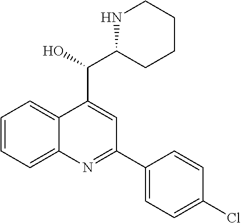 [SMILES: O[C@@H](c1cc(-c2ccc(Cl)cc2)nc2ccccc12)[C@H]1CCCCN1] | (S)-(2-(4-chlorophenyl)quinolin-4- yl)((R)-piperidin-2-yl)methanol |
| S22 | 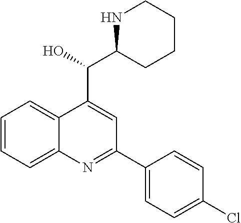 [SMILES: O[C@@H](c1cc(-c2ccc(Cl)cc2)nc2ccccc12)[C@@H]1CCCCN1] | (S)-(2-(4-chlorophenyl)quinolin-4- yl)((S)-piperidin-2-yl)methanol |
| S23 | 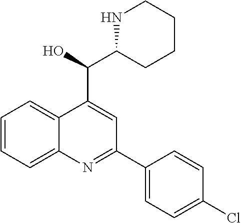 [SMILES: O[C@H](c1cc(-c2ccc(Cl)cc2)nc2ccccc12)[C@H]1CCCCN1] | (R)-(2-(4-chlorophenyl)quinolin-4- yl)((R)-piperidin-2-yl)methanol |
| S24 | 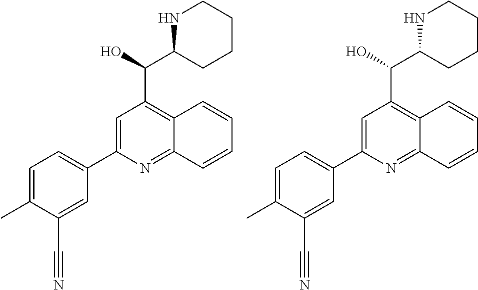 [SMILES: Cc1ccc(-c2cc([C@@H](O)[C@@H]3CCCCN3)c3ccccc3n2)cc1C#N ; Cc1ccc(-c2cc([C@H](O)[C@H]3CCCCN3)c3ccccc3n2)cc1C#N] | Mixture of 5-(4-((R)-hydroxy((S)- piperidin-2-yl)methyl)quinolin- 2-yl)-2-methylbenzonitrile and 5-(4-((S)- hydroxy((R)-piperidin-2- yl)methyl)quinolin- 2-yl)-2-methylbenzonitrile |
| S25 | 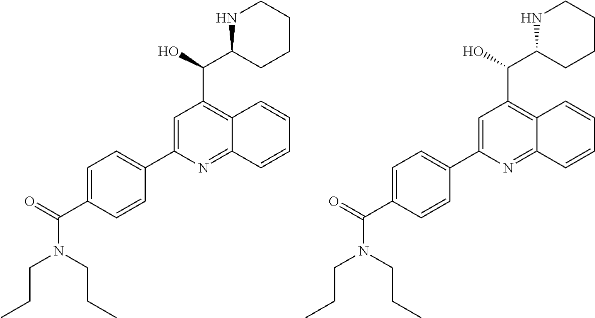 [SMILES: CCCN(CCC)C(=O)c1ccc(-c2cc([C@@H](O)[C@@H]3CCCCN3)c3ccccc3n2)cc1 ; CCCN(CCC)C(=O)c1ccc(-c2cc([C@H](O)[C@H]3CCCCN3)c3ccccc3n2)cc1] | Mixture of 4-(4-((R)-hydroxy((S)- piperidin-2-yl)methyl)quinolin- 2-yl)-N,N-dipropyl- benzamide and 4-(4-((S)- hydroxy((R)-piperidin-2- yl)methyl)quinolin- 2-yl)-N,N-dipropylbenzamide |
| S26 | 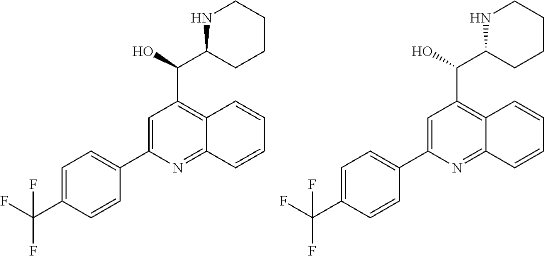 [SMILES: O[C@H](c1cc(-c2ccc(C(F)(F)F)cc2)nc2ccccc12)[C@@H]1CCCCN1 ; O[C@@H](c1cc(-c2ccc(C(F)(F)F)cc2)nc2ccccc12)[C@H]1CCCCN1] | Mixture of (R)-((S)-piperidin- 2-yl)(2-(4-(trifluoro- methyl)phenyl)quinolin-4- yl)methanol and (S)-((R)- piperidin-2-yl)(2- (4-(trifluoromethyl)phenyl) quinolin-4-yl)methanol |
| S27 | 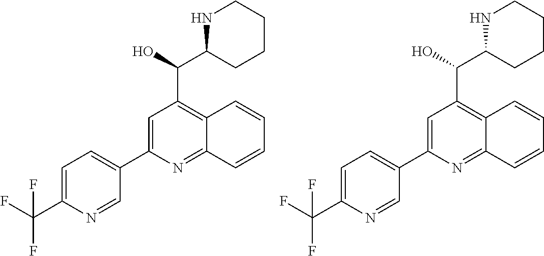 [SMILES: O[C@H](c1cc(-c2ccc(C(F)(F)F)nc2)nc2ccccc12)[C@@H]1CCCCN1 ; O[C@@H](c1cc(-c2ccc(C(F)(F)F)nc2)nc2ccccc12)[C@H]1CCCCN1] | Mixture of (R)-((S)-piperidin- 2-yl)(2-(6-(trifluoromethyl) pyridin-3-yl)quinolin-4- yl)methanol and (S)-((R)- piperidin-2-yl)(2-(6-(trifluoro- methyl)pyridin-3-yl)quinolin-4- yl)methanol |
| S28 | 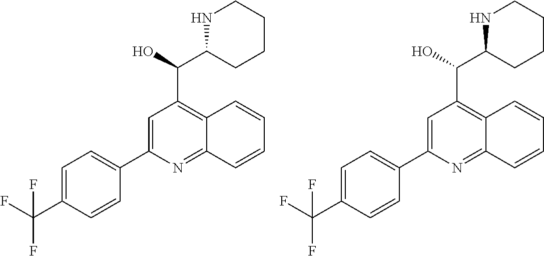 [SMILES: O[C@H](c1cc(-c2ccc(C(F)(F)F)cc2)nc2ccccc12)[C@H]1CCCCN1 ; O[C@@H](c1cc(-c2ccc(C(F)(F)F)cc2)nc2ccccc12)[C@@H]1CCCCN1] | Mixture of (R)-((R)-piperidin- 2-yl)(2-(4-(trifluoromethyl) phenyl)quinolin-4-yl)methanol and (S)-((S)-piperidin-2-yl)(2- (4-(trifluoromethyl)phenyl) quinolin-4-yl)methanol |
| S29 | 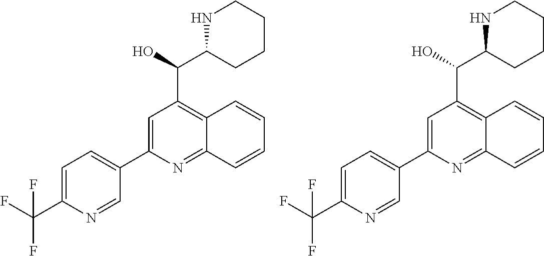 [SMILES: O[C@H](c1cc(-c2ccc(C(F)(F)F)nc2)nc2ccccc12)[C@H]1CCCCN1 ; O[C@@H](c1cc(-c2ccc(C(F)(F)F)nc2)nc2ccccc12)[C@@H]1CCCCN1] | Mixture of (R)-((R)-piperidin- 2-yl)(2-(6-(trifluoromethyl) pyridin-3-yl)quinolin-4- yl)methanol and (S)-((S)- piperidin-2-yl)(2-(6- (trifluoromethyl)pyridin-3- yl)quinolin-4-yl)methanol |

*Compounds provided by the NCI/DTP Open Chemical Repository.

The term “about” is used herein to mean approximately, in the region of, roughly or around. When the term “about” is used in conjunction with a numerical range, it modifies that range by extending the boundaries above and below the numerical values set forth. In general, the term “about” is used herein to modify a numerical value above and below the stated value by a variance of 20%.

As used in the present disclosure, whether in a transitional phrase or in the body of a claim, the terms “comprise(s)” and “comprising” are to be interpreted as having an open-ended meaning. That is, the terms are to be interpreted synonymously with the phrases “having at least” or “including at least.” When used in the context of a process the term “comprising” means that the process includes at least the recited steps, but may include additional steps. When used in the context of a molecule, compound, or composition, the term “comprising” means that the compound or composition includes at least the recited features or components, but may also include additional features or components.

All percentages and ratios used herein, unless otherwise indicated, are by weight. Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

### EXAMPLES

Unless otherwise noted, all solvents and reagents were obtained from commercial sources and used without further purification or characterization. All reactions involving air- or moisture-sensitive reagents were performed under a nitrogen or argon atmosphere using oven-dried glassware. Tetrahydrofuran, dichloromethane, toluene, and diethyl ether were dried by refluxing on sodium metal and freshly distilled as per requirement. Unless otherwise indicated, all reactions were performed at ambient temperatures (18-25° C.). Microwave-assisted reactions were performed in a BIOTAGE, Model: Initiator Exp. EU 355301, 011594-50X. Reactions were magnetically stirred and monitored by thin layer chromatography using TLC silica gel 60 F 254 aluminum sheets from Merck and analyzed with 254 nm UV light and ninhydrin char. Flash chromatography was performed with (60-120 mesh, pH=6.5-7.5) silica gel from Merck. Preparative HPLC was performed on a Gilson 305 HPLC system using either a basic or an acidic eluating protocol. For purification under basic conditions the Gilson 305 HPLC system was equipped with an Xbridge C18 (5 μm, 30 mm×75 mm) column and the compounds were eluted using a gradient system of acetonitrile and H₂O containing 50 mM NH₄HCO₃ (pH 10). For the acidic purification the Gilson 305 HPLC system was equipped with an ACE 5 C8 (5 μm, 30 mm×150 mm) column and the compounds were eluted using a gradient system of acetonitrile and H₂O containing 0.1% TFA. Proton nuclear magnetic resonance (¹H NMR) spectra were recorded using an internal deuterium lock at ambient temperature on a Bruker Avance-III 500 MHz system using Topspin-3 software or a Bruker Avance-I DPX 400 MHz system using Topspin-1 software. All final compounds were purified to ≧95% purity as determined by LCMS or HPLC/UPLC. Compounds were deemed to be pure if the peak area of the compound was >95% of the total peak areas of the UV and LCMS/UPLC chromatograms and if the MS spectra produced the expected m/z and isotopic ratios.

The following compounds were provided by the NCI/DTP Open Chemical Repository: NSC13480 (S1), NSC305787 (S2), NSC157571 (S3), NSC4377 (S4), NSC305758 (S5), NSC14224 (S6), NSC2450 (S7), NSC13316 (S10), NSC16001 (S11), NSC23924 (S12), NSC13097 (S13), NSC23925 (S15), NSC322661 (S17), and NSC13466 (S18). Three general methods for preparing compounds according to formula (I) additionally are illustrated in Examples 1, 2 and 9. The novel compounds S8, S9, S14, S16, S20, S21, S22, and S23, S24, S25, S27, S29 as well as compounds S26 and S28 (described by León, B. et al Org. Lett. 2013, 15, 1234-1237) were prepared as described in Examples 3 to 9. A stereoselective synthesis of S20 is presented in Example 10.

### Example 1

### Synthesis of Vacquinol-1 (S10, NSC13316). General Method A

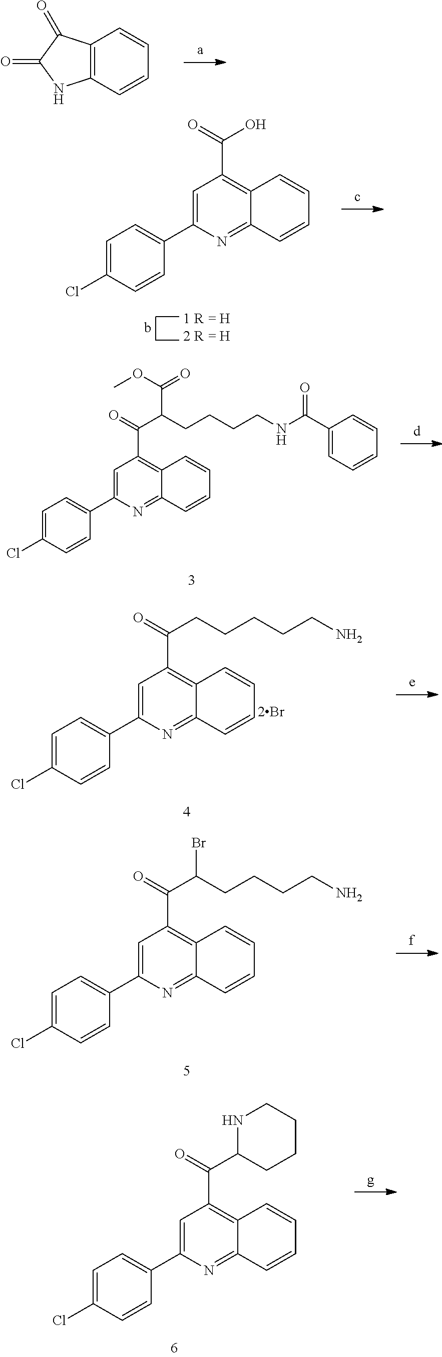 [SMILES: O=C1Nc2ccccc2C1=O ; O=C(O)c1cc(-c2ccc(Cl)cc2)nc2ccccc12 ; COC(=O)C(CCCCNC(=O)c1ccccc1)C(=O)c1cc(-c2ccc(Cl)cc2)nc2ccccc12 ; NCCCCCC(=O)c1cc(-c2ccc(Cl)cc2)nc2ccccc12 ; NCCCCC(Br)C(=O)c1cc(-c2ccc(Cl)cc2)nc2ccccc12 ; O=C(c1cc(-c2ccc(Cl)cc2)nc2ccccc12)C1CCCCN1]

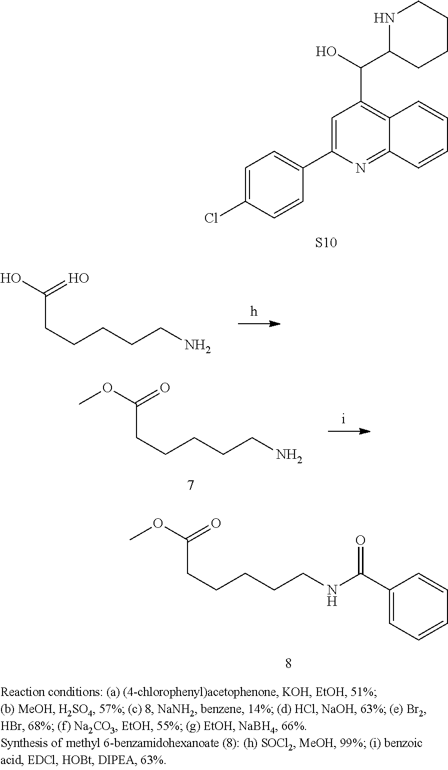

### 2-(4-chlorophenyl)quinoline-4-carboxylic acid (Intermediate 1)

To a stirred solution of isatin (30.0 g, 204 mmol) in 500 mL ethanol, 4-chloroacetophenone (47.0 g, 244 mol) was added in one portion. Potassium hydroxide flakes (22.8 g, 408 mmol) were added in several portions and the reaction was heated to reflux for 14 hr. The reaction was diluted with 1 liter water and washed with ethyl acetate (3×300 mL). The aqueous layer was cooled in an ice-bath and acidified with glacial acetic acid. The precipitated product was filtered, washed with cold, dilute acetic acid and dried in vacuum to give analytically pure intermediate 1 (29.5 g, 51%). TLC: 30% EtOAc/Hexanes (R_{f}: 0.2) ¹H NMR (400 MHz, DMSO-d₆) δ8.59 (d, J=8.6 Hz, 1H), 8.37 (s, 1H), 8.23 (d, J=8.5 Hz, 2H), 8.11 (d, J=8.5 Hz, 1H), 7.82 (t, J=7.7 Hz, 1H), 7.68 (t, J=7.7 Hz, 1H), 7.57 (d, J=8.3 Hz, 2H). LC-MS (ESI⁺): m/z 284.5 [M+H]⁺.

### Methyl 2-(4-chlorophenyl)quinoline-4-carboxylate (Intermediate 2)

To a stirred solution of 1 (500 mg, 1.76 mmol) in MeOH (10 mL), conc. sulphuric acid (0.45 mL) was added. The reaction mixture was heated to reflux for 6 h. The reaction mixture was diluted with saturated NaHCO₃ solution (20 mL) and extracted with EtOAc (2×20 mL). The combined organic extracts were washed with water (20 mL), dried over anhydrous sodium sulfate, filtered and concentrated under reduced pressure to provide the crude material, which was purified by silica gel column chromatography (10% EtOAc/hexanes) to afford intermediate 2 (402 mg, 76%) as an off-white solid. ¹H NMR (400 MHz, CDCl₃): δ8.73 (d, J=8.0 Hz, 1H), 8.35 (s, 1H), 8.26-8.14 (m, 3H), 7.78 (t, J=6.8 Hz, 1H), 7.63 (t, J=7.2 Hz, 1H), 7.50 (d, J=6.8 Hz, 2H), 4.07 (s, 3H). LC-MS (ESI⁺): m/z 298.3 [M+H]⁺.

### Methyl 6-benzamido-2-(2-(4-chlorophenyl)quinoline-4-carbonyl)hexanoate (Intermediate 3)

To a solution of sodium amide (3.10 g, 0.08 mol) in benzene (100 mL) at room temperature, intermediate 2 (10.0 g, 0.03 mol) was added. The reaction mixture was stirred for 10 min and methyl 6-benzamidohexanoate (Intermediate 8, 9.6 g, 0.038 mol) was added. The reaction mixture was stirred for 24 hr at 90° C. The reaction mixture was evaporated to dryness and diluted with water. The crude compound was extracted with EtOAc. The organic layer was dried over sodium sulfate and evaporated under reduced pressure to give intermediate 3. (2.04 g, 13.9%). TLC: 40% EtOAc/Hexanes (R_{f}: 0.1) ¹H NMR (400 MHz, DMSO-d₆) δ8.58 (s, 1H), 8.49-8.29 (m, 3H), 8.19-8.04 (m, 2H), 7.90-7.75 (m, 3H), 7.74-7.55 (m, 3H), 7.47 (m, 3H), 4.96 (m, 1H), 3.92 (m, 1H), 3.2-3.4 (m, 4H), 1.97 (m, 2H), 1.62 (m, 2H), 1.44 (m, 2H). LC-MS (ESI⁺): m/z 516 [M+H]⁺ (78% purity).

### 6-Amino-1-(2-(4-chlorophenyl)quinolin-4-yl)hexan-1-one (Intermediate 4)

Intermediate 3 (10 g, 0.019 mol) was suspended in 6N HCl (100 mL) and refluxed at 110° C. for 48 hrs. The reaction was monitored by LCMS for the complete conversion of the starting material to the product. The pH of the reaction mixture was adjusted to 10-12 using 10% aqueous sodium hydroxide and the crude product was extracted with chloroform. The organic layer was dried over sodium sulfate and evaporated under reduced pressure to give intermediate 4 (3.94 g) as a crude (63% purity) which was used directly in the next step. TLC: 30% EtOAc/Hexanes (R_{f}: 0.3). ¹H NMR (400 MHz, CDCl₃) δ8.35-7.84 (m, 4H), 7.74 (s, 1H), 7.65-7.33 (m, 4H), 4.00 (s, 1H), 3.18-2.78 (m, 1H), 1.90 (d, J=74.6 Hz, 2H), 1.38-0.69 (m, 5H). LC-MS (ESI⁺): m/z 353 [M+H]⁺.

### 6-Amino-2-bromo-1-(2-(4-chlorophenyl)quinolin-4-yl)hexan-1-one hydrobromide (Intermediate 5)

Intermediate 4 (3.0 g, 8.5 mmol) was dissolved in chloroform (50 mL) and hydrobromic acid (47% aq. solution, 20 mL) was added. The reaction mixture was allowed to stir at room temperature for 30 min. The solvent was removed under reduced pressure and the suspension was heated to 90° C. upon which bromine (1.35 g, 8.52 mmol) was added to the reaction mixture over 20 min. The reaction mixture was cooled to room temperature and diluted with water (50 mL). The obtained solid was filtered and washed with several portions diethyl ether. The crude intermediate 5 (2.47 g, 68.3%) obtained was used next step without further purification. TLC: 30% EtOAc/Hexanes (R_{f}: 0.1) ¹H NMR (400 MHz, DMSO-d₆) δ8.62 (s, 1H), 8.42 (d, J=8.3 Hz, 2H), 8.18 (d, J=8.2 Hz, 1H), 8.07 (d, J=8.4 Hz, 1H), 7.89 (m, 1H), 7.70 (m, 5H), 6.01 (m, 1H), 2.86 (m, 2H), 2.27 (m, 1H), 2.08 (m, 1H), 1.87-1.47 (m, 4H). LC-MS (ESI⁺): m/z 433 [M+H]⁺ (52.6% purity).

### (2-(4-Chlorophenyl)quinolin-4-yl) (piperidin-2-yl)methanone (Intermediate 6)

Crude 6-amino-2-bromo-1-(2-(4-chlorophenyl)quinolin-4-yl)hexan-1-one hydrobromide (intermediate 5, 2.50 g, 5.81 mmol) was dissolved in ethanol (60 mL) and 15% sodium carbonate solution (20 mL) was added to it. The reaction was stirred for 1 hr. TLC showed complete conversion of the starting material. The reaction mixture was filtered through a Buchner funnel and the ethanol layer was evaporated under reduced pressure. The crude compound was purified by column chromatography using 15% ethyl acetate in hexane with 100-200 mesh silica gel to yield intermediate 6 (1.1 g, 55%). TLC: 30% EtOAc/Hexanes (R_{f}: 0.5) ¹H NMR (400 MHz, Methanol-d₄) δ8.19 (d, J=8.3 Hz, 2H), 8.15 (d, J=8.3 Hz, 1H), 7.91 (s, 1H), 7.85-7.75 (m, 2H), 7.60 (d, J=7.6 Hz, 1H), 7.55 (d, J=8.3 Hz, 2H), 5.31 (m, 1H), 3.28 (m, 2H), 2.24 (m, 2H), 1.87 (m, 2H), 1.30 (m, 2H). LC-MS (ESI⁺): m/z 352 [M+H]⁺ (99.4% purity).

### (2-(4-chlorophenyl)quinolin-4-yl) (piperidin-2-yl)methanol (S10, Vacquinol-1, NSC13316)

Intermediate 6 (3.0 g, 8.5 mmol) was dissolved in ethanol under nitrogen atmosphere. The reaction mixture was cooled to 0° C. and sodium borohydride (108 mg, 17.1 mmol) was added in portions to the reaction mixture. The reaction was stirred for 60 min at 0° C. and monitored by TLC. The reaction was quenched with water (5 mL) and solvents evaporated under reduced pressure. The crude was distributed between with ethyl acetate and water and the organic layer was washed with water, dried over anhydrous sodium sulfate and concentrated under reduced pressure. The crude compound was purified by column chromatography using 15% ethyl acetate in hexane as the eluent to give desired product S10 (NSC13316) (2.06 g, 66.3%). TLC: 30% EtOAc/Hexanes (R_{f}: 0.2) ¹H NMR (400 MHz, DMSO-d₆) δ8.27 (m, 3H), 8.15 (d, J=6.1 Hz, 1H), 8.09 (d, J=8.6 Hz, 1H), 7.77 (t, J=7.7 Hz, 1H), 7.62 (d, J=8.3 Hz, 3H), 5.73 (m, 1H), 5.33-5.03 (m, 1H), 3.46-3.20 (m, 1H), 3.07-2.76 (m, 2H), 2.42 (m, 1H), 1.77-0.98 (m, 6H). LC-MS (ESI⁺): m/z 353 [M+H]⁺ (99.4% purity).

### Methyl 6-aminohexanoate (Intermediate 7)

To a stirred solution of 6-aminocaproic acid (50.0 g, 0.38 mol) in dry methanol (650 mL) under nitrogen atmosphere, thionyl chloride (47.6 g, 0.40 mol) was added dropwise at 0° C. The reaction mixture was stirred for 10 min and then refluxed at 90° C. for 3 h. After the completion of the reaction, solvent was evaporated to dryness and a white solid was obtained. The obtained solid was washed with hexane to give 69 g of the desired intermediate 7. (Yield: 99%). TLC: 10% MeOH/DCM (R_{f}: 0.2) ¹H NMR (400 MHz, DMSO-d₆) δ8.11 (s, 3H), 3.66-3.50 (s, 3H), 2.72 (m, 2H), 2.29 (t, J=7.3 Hz, 2H), 1.54 (m, 4H), 1.30 (m, 2H). LC-MS (ESI⁺): m/z 146 [M+H]⁺ (100% purity).

### Methyl 6-benzamidohexanoate (Intermediate 8)

To a solution of methyl 6-aminohexanoate (intermediate 7, 17.8 g, 0.098 mol) in DMF (150 mL), N-(3-dimethylaminopropyl)-N′-ethylcarbodiimide hydrochloride (19.05 g, 0.122 mol), hydroxybenzotriazole (16.47 g, 0.122 mol) and diisopropyl ethyl amine (31.72 g, 0.245 mol) were added. The reaction mixture was stirred for 10 min and benzoic acid (10 g, 0.08 mol) was added. The reaction mixture was stirred overnight at room temperature, then diluted with water and extracted with ethyl acetate. The combined organic layers were combined, dried over anhydrous sodium sulfate and evaporated under reduced pressure to afford the desired intermediate 8. (12.76 g, 62.5%). TLC: 10% MeOH/DCM (R_{f}: 0.5) ¹H NMR (400 MHz, DMSO-d₆) δ8.42 (d, J=6.0 Hz, 1H), 7.83 (d, J=7.3 Hz, 2H), 7.47 (m, 3H), 3.57 (s, 3H), 3.24 (m, 2H), 2.30 (t, J=7.4 Hz, 2H), 1.54 (m, 4H), 1.30 (m, 2H). LC-MS (ESI⁺): m/z 250 [M+H]⁺ (66% purity).

### Example 2

### Synthesis of Vacquinol-1 (S10, NSC13316). General Method B

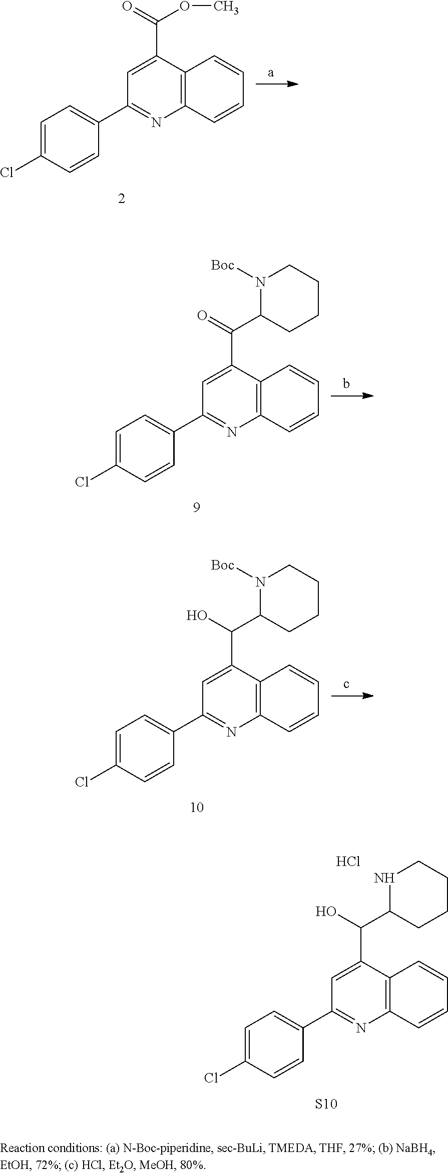 [SMILES: COC(=O)c1cc(-c2ccc(Cl)cc2)nc2ccccc12 ; CC(C)(C)OC(=O)N1CCCCC1C(=O)c1cc(-c2ccc(Cl)cc2)nc2ccccc12 ; CC(C)(C)OC(=O)N1CCCCC1C(O)c1cc(-c2ccc(Cl)cc2)nc2ccccc12 ; Cl ; OC(c1cc(-c2ccc(Cl)cc2)nc2ccccc12)C1CCCCN1 ; "S10"]

### tert-Butyl 2-(2-phenylquinoline-4-carbonyl) piperidine-1-carboxylate (Intermediate 9)

To a stirred solution of tert-butyl piperidine-1-carboxylate (1.0 g, 5.4 mmol) in dry THF (30 mL), cooled to 0° C., TMEDA (2 mL) and sec-butyl lithium (1.4 M in cyclohexane, 5 mL, 7.06 mmol) were added drop-wise and stirred for 2 h. A solution of compound 2 (1.42 g, 5.43 mmol) in dry THF (30 mL) was added to the reaction mixture and stirring continued further 2 h at 0° C. The reaction mixture was slowly warmed to RT and stirred for 3 h (monitored by TLC). After complete consumption of the starting material; the reaction mixture was quenched with saturated ammonium chloride solution (40 mL) and extracted with EtOAc (2×40 mL). The combined organic extracts were washed with water (40 mL), dried over sodium sulfate, filtered and concentrated under reduced pressure to obtain the crude residue. The crude material was purified by silica gel column chromatography (5% EtOAc/Hexanes) to afford intermediate 9 (600 mg, 27%) as a yellow solid. TLC: 5% EtOAc/Hexanes (R_{f}: 0.4) ¹H NMR (400 MHz, CD₃OD): δ 8.3-8.15 (m, 5H), 7.94-7.81 (m, 1H), 7.68-7.61 (m, 1H), 7.60-7.55 (m, 2H), 5.72-5.68 (m, 1H), 4.02-3.92 (m, 1H), 3.05-2.97 (m, 1H), 2.18-2.05 (m, 2H), 1.78-1.65 (m, 4H), 1.38 (s, 9H). LC-MS (ESI⁺): m/z 451 [M+1] at 5.21 RT (87.19% purity); HPLC Purity: 81.76%.

### tert-Butyl 2-((2-(4-chlorophenyl) quinolin-4-yl) (hydroxy) methyl) piperidine-1-carboxylate (intermediate 10)

To a stirred solution of intermediate 9 (400 mg, 0.96 mmol) in EtOH (8 mL), cooled to 0° C., NaBH₄ (72 mg, 1.92 mmol) was added and the reaction stirred for 2 h. After complete consumption of the starting material, the reaction was diluted with water (20 mL) and extracted with EtOAc (2×20 mL). The combined organic extracts were washed with water (20 mL), dried over sodium sulfate, filtered and concentrated under reduced pressure to obtain the crude material, which was purified by column chromatography (silica gel, 15% EtOAc/Hexanes) to afford intermediate 10 (racemic) (180 mg, 72%) as an off-white solid. TLC: 30% EtOAc/Hexanes (R_{f}: 0.25). ¹H NMR (400 MHz, CD₃OD): (Racemic) δ 8.6-8.35 (m, 1H), 8.15-8.05 (m, 4H), 7.81-7.72 (m, 1H), 7.75-7.51 (m, 3H), 5.8-5.6 (m, 1H), 4.7-4.55 (m, 1H), 4.11-3.95 (m, 1H), 3.44-3.15 (m, 1H), 1.95-1.41 (m, 6H), 1.34 (s, 9H). LC-MS (ESI⁺): m/z 453.5 [M+H]⁺. UPLC Purity: 28.03% and 68.16% (Racemic)

### (2-(4-chlorophenyl) quinolin-4-yl) (piperidin-2-yl) methanol hydrochloride (S10, NSC13316)

To a stirred solution of intermediate 10 (80 mg, 0.17 mmol) in MeOH (2 mL), cooled to 0° C., 4 M HCl in ether (0.17 mL, 3.53 mmol) was added at 0° C. The reaction mixture was warmed to RT and stirred for 4 h (monitored by TLC). After complete consumption of the starting material; the volatiles were evaporated under reduced pressure and the crude material was triturated with ether (2×10 mL) to afford compound S10 (Vacquinol-1, NSC13316) (50 mg, 80%) as an off-white solid. TLC: 40% EtOAc/Hexanes (R_{f}: 0.1) ¹H NMR (400 MHz, CD₃OD-d₄): (Racemic) δ 8.56-8.45 (m, 2H), 8.39 (d, J=8.8 Hz, 1H), 8.20-8.12 (m, 3H), 8.02-7.94 (m, 1H), 7.77-7.74 (m, 2H), 6.05-5.7 (m, 1H), 3.73-3.64 (m, 1H), 3.48-3.40 (m, 1H), 3.18-3.12 (m, 1H), 2.99-2.94 (m, 1H), 1.90-1.80 (m, 4H), 1.52-1.29 (m, 2H). LC-MS: (Racemic) 54.37% at 4.28 RT, 43.27% at 4.37 RT; 353.3 (M+1) UPLC (purity): (Racemic) 64.25%+33.21%. LC-MS (ESI⁺): m/z 353 [M+H]⁺

### Example 3

### Synthesis of S14

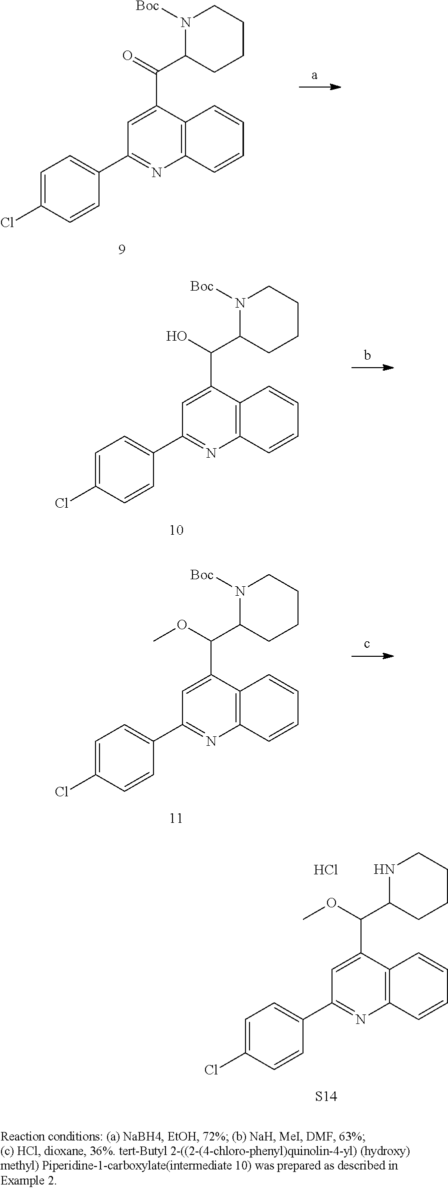 [SMILES: CC(C)(C)OC(=O)N1CCCCC1C(=O)c1cc(-c2ccc(Cl)cc2)nc2ccccc12 ; CC(C)(C)OC(=O)N1CCCCC1C(O)c1cc(-c2ccc(Cl)cc2)nc2ccccc12 ; COC(c1cc(-c2ccc(Cl)cc2)nc2ccccc12)C1CCCCN1C(=O)OC(C)(C)C ; COC(c1cc(-c2ccc(Cl)cc2)nc2ccccc12)C1CCCCN1 ; Cl ; "S14"]

### tert-Butyl 2-((2-(4-chlorophenyl) quinolin-4-yl) (methoxy) methyl) piperidine-1-carboxylate (intermediate 11)

To a stirred solution of intermediate 10 (140 mg, 0.31 mmol) in DMF (1 mL), cooled to 0° C., sodium hydride (18.5 mg, 0.46 mmol) was added under inert atmosphere and stirred for 10 min. Methyl iodide (0.023 mL, 0.371 mmol) was added to the reaction mixture which was slowly warmed to RT and stirred for 1 h (monitored by TLC). After complete consumption of the starting material, the reaction mixture was diluted with water (10 mL) and extracted with EtOAc (2×25 mL). The combined organic extracts were washed with water (50 mL), dried over sodium sulfate, filtered and concentrated under reduced pressure. The crude residue was purified by silica gel column chromatography (5-10% EtOAc/hexanes) to afford intermediate 11 (90 mg, 62.5%) as a colorless thick syrup. Used without further purification TLC: 1:3 EtOAc:hexanes (R_{f}: 0.6) LC-MS (ESI⁺): (Racemic) m/z 467.6 [M+H]⁺. HPLC (purity): (Racemic) 61.37% purity at 16.51 RT and 32.75% purity at 16.14 RT.

### 2-(4-Chlorophenyl)-4-(methoxy (piperidin-2-yl) methyl) quinoline hydrochloride (S14)

To a stirred solution of intermediate 11 (90 mg, 0.19 mmol) in MeOH (2 mL), cooled to 0° C., 4 N HCl in dioxane (0.2 mL, 0.77 mmol) was added drop-wise under inert atmosphere and stirred for 16 h. The progress of the reaction was monitored by TLC. After complete consumption of the starting material, the volatiles were evaporated in vacuo to obtain the crude material which was purified by preparative HPLC-MS to afford S14 (25 mg, 36%) as a colorless gummy solid. TLC: 1:3 EtOAc:hexanes (R_{f}: 0.1) ¹H NMR (400 MHz, CD₃OD) (racemic): δ8.30 (d, J=8.8 Hz, 1H), 8.23-8.18 (m, 1H), 8.13 (d, J=8.8 Hz, 1H), 8.09 (s, 1H), 8.01 (s, 1H), 7.88-7.84 (m, 1H), 7.71-7.68 (m, 1H), 7.58 (d, J=8.4 Hz, 2H), 5.40-5.07 (m, 1H) 3.70-3.52 (m, 1H), 3.47-3.44 (m, 1H), 3.38 (s, 3H), 3.06-2.99 (m, 1H), 1.91-1.60 (m, 4H), 1.50-1.29 (m, 2H). LC-MS (ESI⁺): (racemic) m/z 367.3 [M+H]⁺. HPLC Purity: (racemic) 63.41% at 15.64 RT and 35.68% at 16.78 RT.

### Example 4

### Synthesis of S19

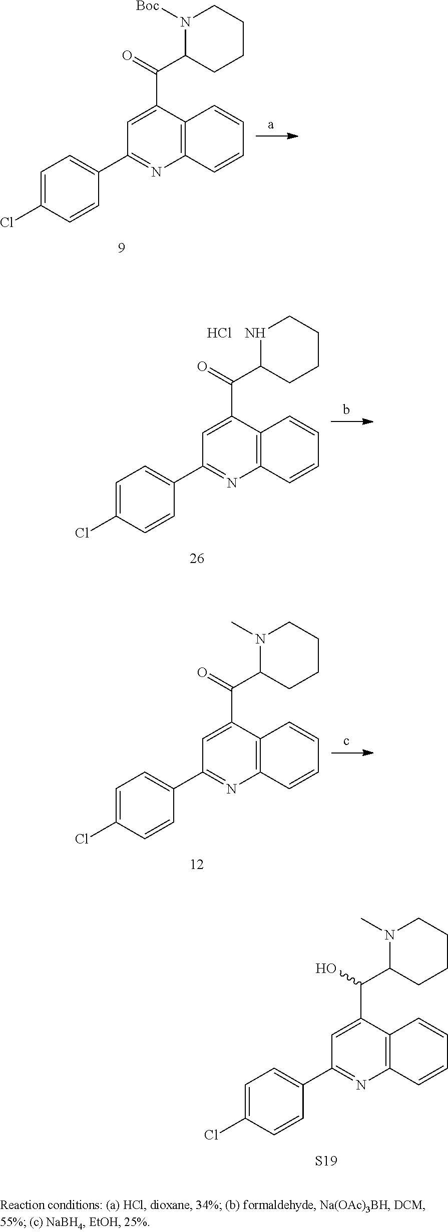

### (2-(4-Chlorophenyl)quinolin-4-yl)(piperidin-2-yl)methanone (intermediate 26)

To a stirred solution of intermediate 9 (150 mg, 0.33 mmol) in CH₂Cl₂ (5 mL), cooled to 0° C., 4 N HCl in 1, 4-dioxane (0.17 mL) was added. The reaction mixture was slowly warmed to RT and stirred for 2 h (monitored by TLC). After complete consumption of the starting material, the volatiles were evaporated under reduced pressure and the residue was triturated with ether (2×10 mL) to obtain the crude material. The crude residue was purified by mass-directed purification to afford intermediate 26 (40 mg, 34%) as off-white solid. TLC: 20% EtOAc/Hexanes (R_{f}: 0.3) ¹HNMR (400 MHz, CD₃OD): δ 8.37 (s, 1H), 8.31 (d, J=6.8 Hz, 2H), 8.29-8.20 (m, 2H), 7.91-7.86 (m, 1H), 7.73-7.69 (m, 1H), 7.61-7.58 (m, 2H), 5.26-5.23 (m, 1H), 3.64-3.61 (m, 1H), 3.21-3.20 (m, 1H), 2.14-2.09 (m, 1H), 1.99-1.91 (m, 2H), 1.78-1.64 (m, 3H). LC-MS: 98.29%; 351 (M+1); (column; X-Bridge C-18 (50×3.0 mm, 3.5 m); RT 3.51 min; 0.05% TFA in water: ACN; 0.80 ml/min). UPLC (purity): 96.23%.

### (2-(4-chlorophenyl) quinolin-4-yl) (1-methylpiperidin-2-yl) methanone (Intermediate 12)

To a stirred solution of intermediate 26 (140 mg, 0.40 mmol) in dichloromethane (10 mL), cooled to 0° C., aq. formaldehyde (37%, 0.1 mL, 1.20 mmol) was added and stirred for 20 min. NaBH(OAc)₃ (169 mg, 0.80 mmol) was added and stirring continued for 2 h (monitored by TLC). After complete consumption of the starting material, the reaction mixture was diluted with water (10 mL) and extracted with DCM (2×10 mL). The combined organic extracts were washed with water (10 mL), dried over sodium sulfate, filtered and concentrated under reduced pressure to obtain the crude residue. The crude material was purified by silica gel column chromatography (15-20% EtOAc/Hexanes) to afford intermediate 12 (80 mg, 55%) as a colorless thick syrup. TLC: 40% EtOAc/Hexanes (R_{f}: 0.5). ¹H NMR (400 MHz, CD₃OD): δ8.29-8.28 (m, 2H), 8.20-8.13 (m, 2H), 7.84 (d, J=7.5 Hz, 1H), 7.69 (d, J=7.5 Hz, 1H), 7.67-7.58 (m, 3H), 4.55 (m, 1H), 3.35 (s, 3H), 2.60 (m, 2H), 1.88-1.85 (m, 3H), 1.81-1.78 (m, 3H), 1.57-1.53 (m, 2H). LC-MS (ESI⁺): m/z 365 [M+H]⁺ 74.13% (purity) at 4.53 RT; UPLC Purity: 77.47%

### (2-(4-Chlorophenyl) quinolin-4-yl) (1-methylpiperidin-2-yl)methanol (S19)

To a stirred solution of intermediate 12 (80 mg, 0.21 mmol) in EtOH (2 mL), cooled to 0° C., NaBH₄ (17 mg, 0.44 mmol) was added and the reaction stirred for 1 h (monitored by TLC). After complete consumption of the starting material, the reaction mixture was diluted with water (10 mL) and extracted with EtOAc (2×10 mL). The combined organic extracts were washed with water (10 mL), brine (10 mL), dried over sodium sulfate, filtered and concentrated under reduced pressure to obtain the crude material which was purified by preparative HPLC to afford S19 (20 mg, 25%) as off-white colored solid. TLC: 40% EtOAc/Hexanes (R_{f}: 0.1) ¹H NMR (400 MHz, CD₃OD): (Racemic) δ8.26 (s, 1H), 8.22-8.17 (m, 3H), 8.20-8.18 (m, 1H), 8.13-8.10 (d, J=8.4 Hz, 1H), 7.86 (t, J=8.0 Hz, 1H), 7.73 (t, J=8.0 Hz, 1H), 7.59 (d, J=6.8 Hz, 2H), 6.20-6.18 (m, 1H), 3.72-3.68 (m, 1H), 3.54-3.51 (m, 1H), 3.25 (s, 3H), 3.22-3.21 (m, 1H), 1.93-1.72 (m, 4H), 1.33-1.26 (m, 1H), 1.16-1.13 (m, 1H). LC-MS (ESI⁺): m/z 367.4 [M+H]. UPLC Purity: (Racemic) 78.21% at 1.99 RT and 17.75% at 2.02 RT

### Example 5

### Synthesis of S16

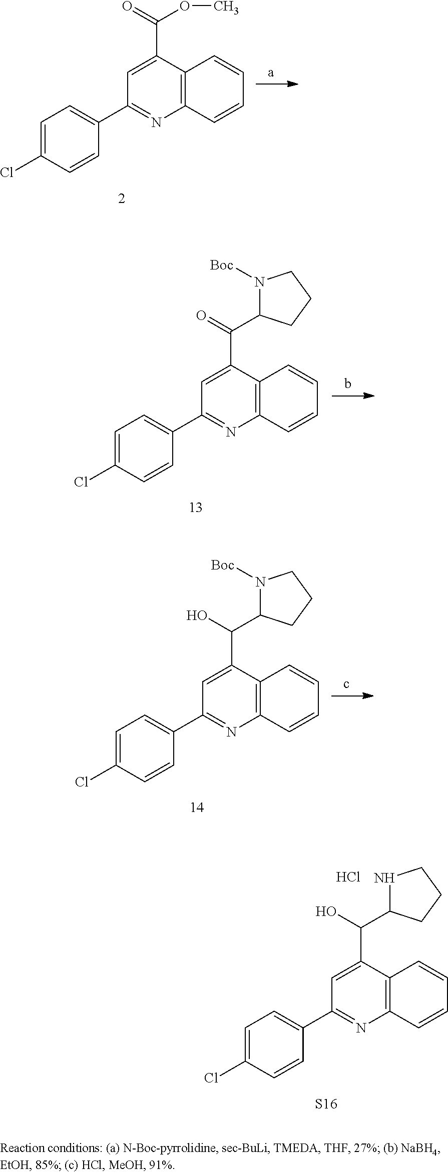 [SMILES: COC(=O)c1cc(-c2ccc(Cl)cc2)nc2ccccc12 ; CC(C)(C)OC(=O)N1CCCC1C(=O)c1cc(-c2ccc(Cl)cc2)nc2ccccc12 ; CC(C)(C)OC(=O)N1CCCC1C(O)c1cc(-c2ccc(Cl)cc2)nc2ccccc12 ; OC(c1cc(-c2ccc(Cl)cc2)nc2ccccc12)C1CCCN1 ; Cl ; "S16"]

### tert-Butyl 2-(2-(4-chlorophenyl) quinoline-4-carbonyl) pyrrolidine-1-carboxylate (intermediate 13)

To a stirred solution of tert-butyl pyrrolidine-1-carboxylate (500 mg, 2.94 mmol) in dry THF (10 mL), cooled to −78° C., TMEDA (1 mL, cat) followed by sec-BuLi (1.4 M in cyclohexane, 2.73 mL, 3.82 mmol) were added and stirred for 2 h. A solution of 2 (873 mg, 2.94 mmol) in dry THF (5 mL) was added to the reaction mixture maintaining the temperature at −78° C. and continued for further 1 h. The reaction mixture was slowly warmed to RT, stirred for 2 h (monitored by TLC) and quenched with saturated NH₄Cl solution (20 mL). The reaction mixture was extracted with EtOAc (2×25 mL) and the combined organic extracts were washed with water (20 mL), dried over sodium sulfate, filtered and concentrated under reduced pressure to obtain the crude material. The crude residue was purified by silica gel column chromatography (10% EtOAc/Hexanes) to afford intermediate 13 (350 mg, 27%) as a colorless thick syrup. TLC: 5% EtOAc/Hexanes (R_{f}: 0.4) ¹H NMR (400 MHz, CD₃OD): δ8.31-8.16 (m, 5H), 7.86-7.81 (m, 1H), 7.69-7.63 (m, 1H), 7.59-7.56 (m, 2H), 5.44-5.41 (m, 1H), 3.65-3.49 (m, 2H), 2.36-2.20 (m, 1H), 2.01-1.98 (m, 3H), 1.28 (s, 9H). LC-MS: m/z 437.5 [M+H]⁺ at 4.87 RT (95.27% purity). UPLC Purity: 95.51%

### tert-Butyl 2-((2-(4-chlorophenyl) quinolin-4-yl) (hydroxy) methyl) pyrrolidine-1-carboxylateoxylate (intermediate 14)

To a stirred solution of 13 (200 mg, 0.45 mmol) in EtOH (5 mL), cooled to 0° C., NaBH₄ (34.6 mg, 0.44 mmol) was added portion-wise and stirred for 1 h (monitored by TLC). After complete consumption of the starting material the reaction was diluted with water (15 mL) and extracted with EtOAc (2×15 mL). The combined organic extracts were washed with water (15 mL), dried over sodium sulfate, filtered and concentrated under reduced pressure to obtain the crude residue. The crude material was purified by silica gel column chromatography (20% EtOAc/Hexanes) to afford intermediate 14 (170 mg, 85%) as an off-white solid. TLC: 30% EtOAc/Hexanes (R_{f}: 0.4). ¹H NMR (400 MHz, CD₃OD): (Racemic) 8.19-8.10 (m, 5H), 7.78-7.75 (m, 1H), 7.56-7.55 (m, 3H), 6.08-5.82 (m, 1H), 4.44-4.43 (m, 1H), 3.60-3.40 (m, 1H), 3.21-3.15 (m, 1H), 2.25-2.23 (m, 2H), 2.12-2.11 (m, 2H), 1.45 (s, 9H). LC-MS: (Racemic) 62.59% at 4.68 RT, 36.11% at 4.87 RT; 439.5 [M+H]⁺. HPLC Purity: (Racemic) 67.22% at 12.53 RT, 31.72% at 13.20 RT.

### (2-(4-Chlorophenyl) quinolin-4-yl) (pyrrolidin-2-yl) methanol hydrochloride (S16)

To a stirred solution of intermediate 14 (170 mg, 0.38 mmol) in MeOH (4 mL), cooled to 0° C., 2 M HCl in ether (0.38 mL, 1.55 mmol) was added. The reaction mixture was warmed to RT and stirred for 16 h (monitored by TLC). After complete consumption of the starting material, the volatiles were evaporated under reduced pressure and the crude residue was triturated with ether (2×10 mL) to afford S16 (120 mg, 91%) as an off-white solid. TLC: 60% EtOAc/Hexanes (R_{f}: 0.2) ¹H NMR (400 MHz, CD₃OD): (Racemic) δ8.64-8.58 (m, 1H), 8.55 (s, 1H), 8.46 (d, J=8.8 Hz, 1H), 8.23-8.16 (m, 3H), 8.08-8.02 (m, 1H), 7.79 (d, J=8.4 Hz, 2H), 6.22-6.21 (m, 1H), 6.03-6.02 (m, 1H), 4.22-4.20 (m, 1H), 4.05-4.04 (m, 1H), 3.43-3.39 (m, 1H), 3.26-3.20 (m, 1H), 2.33-2.11 (m, 3H), 1.95-1.93 (m, 1H), 1.60-1.5 (m, 1H). LC-MS: (Racemic) 56.61% at 3.84 RT, 42.80% at 3.98 RT; 339.2 (M+1); (column; X-Bridge C-18 (50×3.0 mm, 3.5 μm); 5 mM NH4OAc: ACN; 0.80 ml/min); UPLC (purity): (Racemic) 65.82% at 1.87 RT, 32.19% at 1.93 RT.

### Example 6

### Synthesis of S9

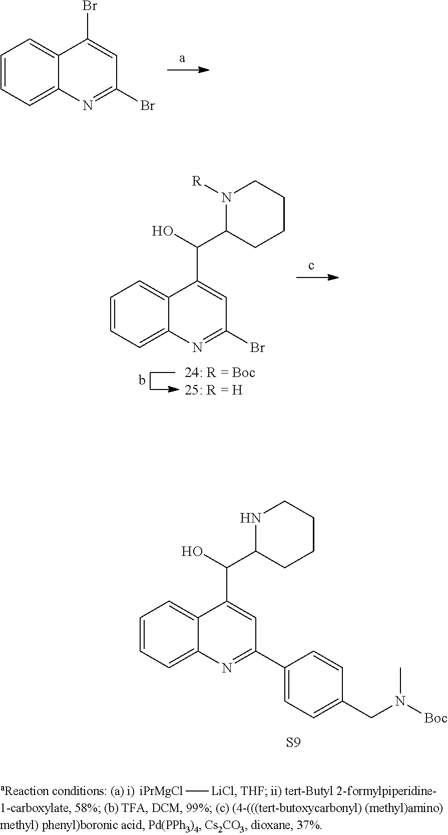

### tert-Butyl 2-((2-bromoquinolin-4-yl) (hydroxy)methyl)piperidine-1-carboxylate (intermediate 24)

2,4-Dibromoquinoline (500 mg, 1.74 mmol) was dissolved in dry THF (6 mL). i-PrMgCl LiCl (1.47 mL, 1.3M, 1.9 mmol) was added slowly, dropwise, at room temperature followed by the addition of N-boc piperidine-2-aldehyde (483.1 mg, 2.27 mmol). The mixture was stirred for 4 hrs checking the consumption of the magnesium reagent by LC-MS analysis. After the reaction was complete, sat. NH₄Cl solution was added and the mixture was extracted three times with EtOAc. The solvent was evaporated and the product was purified by flash chromatography (EtOAc/heptane=1/4) and trituration in heptane to yield the intermediate 24 (474 mg, 58%) as a white solid. ¹H NMR (CDCl₃, 400 MHz): δ 8.22 (dd, J=8.5, 0.9 Hz, 1H), 7.97 (dd, J=8.6, 0.8 Hz, 1H), 7.62-7.73 (m, 2H), 7.55 (ddd, J=8.3, 6.9, 1.4 Hz, 1H), 5.66 (t, J=4.5 Hz, 1H), 4.31 (q, J=5.1 Hz, 1H), 3.83 (d, J=13.1 Hz, 1H), 3.71 (br. s., 1H), 3.19 (ddd, J=14.3, 13.1, 4.0 Hz, 1H), 1.87-1.98 (m, 1H), 1.77 (tt, J=9.5, 4.5 Hz, 1H), 1.59 (tt, J=8.1, 4.0 Hz, 1H), 1.42-1.54 (m, 2H), 1.21-1.41 ppm (m, 10H).

### (2-Bromoquinolin-4-yl)(piperidin-2-yl)methanol (intermediate 25)

Intermediate 24 (50 mg, 0.12 mmol) was dissolved in 5 mL DCM and 100 microliters TFA added. After 5 hrs, the reaction was quenched with saturated Na₂CO₃ (pH≈11) and the organic layer was decanted. The aqueous layer was extracted 3 times with DCM and the residue was concentrated under reduced pressure to yield the crude intermediate 25 as white powder which was directly used in the following step without further purification or characterization.

### tert-butyl 4-(4-(hydroxy(piperidin-2-yl)methyl)quinolin-2-yl)benzyl(methyl)carbamate (S9)

A flask was charged with intermediate 25 (38.1 mg, 0.12 mmol), (4-(((tert-butoxycarbonyl)-(methyl)amino)methyl) phenyl)boronic acid (35.6 mg, 0.13 mmol), Pd(PPh3)₄ (13.7 mg, 0.012 mmol) in 1,4-dioxane (790 μL). The flask was degassed three times. To the mixture was added a solution of Cs₂CO₃ (77 mg, 0.24 mmol) in H₂O (500 μL). The flask was degassed again three times. The reaction mixture was stirred at 75° C. for 1 h. After being cooled to rt, water was added and the aqueous layer was extracted three times with EtOAc. The combined organic layers were washed with brine, dried with MgSO₄ and concentrated under reduced pressure. The crude was purified by reversed phase column chromatography to give 20.0 mg (37%) S9 as a white solid. ¹H NMR (CDCl₃, 400 MHz): δ 8.21 (s, 1H), 8.13-8.20 (m, 3H), 7.96 (d, J=8.3 Hz, 1H), 7.66-7.77 (m, 1H), 7.45-7.56 (m, 1H), 7.38 (br. s., 2H), 5.48 (d, J=3.3 Hz, 1H), 4.50 (br. s., 2H), 3.00-3.12 (m, 2H), 2.75-2.98 (m, 3H), 2.69 (td, J=12.1, 2.7 Hz, 1H), 1.45-1.80 (m, 13H), 1.04-1.44 ppm (m, 3H). ¹³C NMR (CDCl₃, 101 MHz): δ 156.7, 148.9, 148.5, 148.4, 147.4, 139.5, 138.8, 130.7, 129.3, 127.8, 126.1, 125.0, 124.7, 123.0, 116.5, 79.8, 72.7, 61.2, 46.9, 41.0, 28.5, 26.1, 25.1, 24.3 ppm.

### Example 7

### Stereoselective Synthesis of S20 and S22

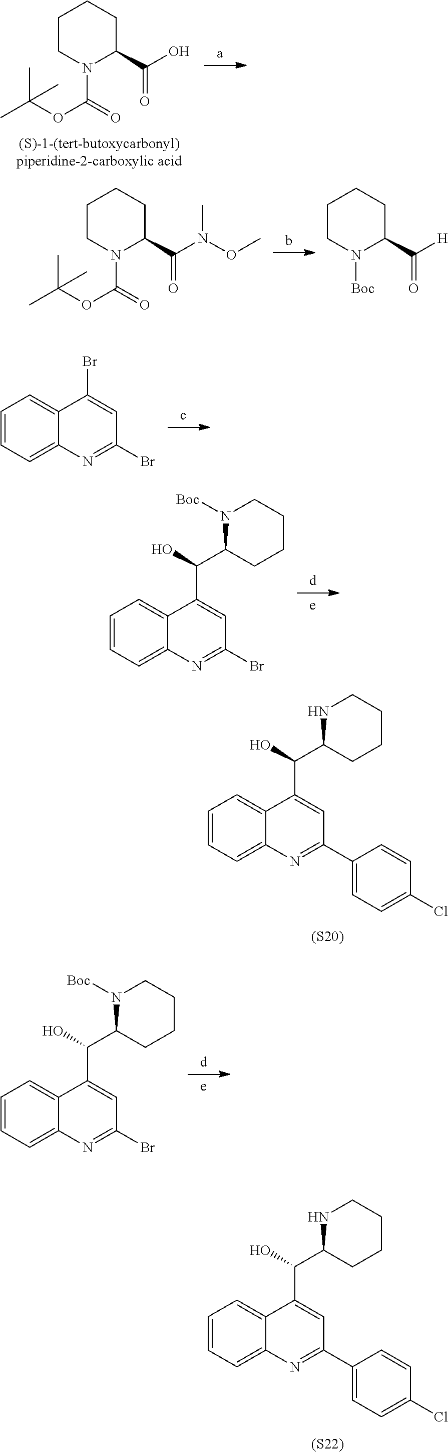

### a) tert-butyl (2S)-2-[methoxy(methyl)carbamoyl]piperidine-1-carboxylate

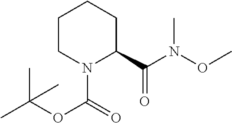 [SMILES: CON(C)C(=O)[C@@H]1CCCCN1C(=O)OC(C)(C)C]

(S)-(L)-N-Boc-Pipecolic acid (0.5 g, 2.2 mmol) was dissolved in DMF (2.4 ml), diisopropylethylamine (2.2 mL, 4.4 mmol) was added followed by HATU (1.2 g, 3.3 mmol) at 22° C. The reaction mixture was stirred for 5 min. N,O-Dimethylhydroxylamine hydrochloride (0.3 g, 3.3 mmol) was added and reaction mixture was stirred at room temperature for 1 h The solution was diluted with EtOAc (20 mL) and poured into 1M HCl (20 ml). The organic phase was separated and washed with saturated aqueous sodium hydrogen carbonate (25 mL) and brine (25 mL) The solution was dried over MgSO₄, filtered and then evaporated in vacuum. The resultant colorless oil was chromatographed on silica gel eluting with 20% ethyl acetate in heptane. Fractions were collected, evaporated and dried under vacuum for 24 h. Yielded the title compound (546 mg, 92%) as a colorless oil. HPLC-MS (API-ES) Exact mass for C13H24N2O4 [M+H]⁺ requires m/z 273.1814. found m/z 273.

### b) tert-butyl (2S)-2-formylpiperidine-1-carboxylate

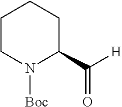 [SMILES: CC(C)(C)OC(=O)N1CCCC[C@H]1C=O]

LiAlH₄ (1M in THF, 3.0 mL, 3.0 mmol) was added in portions to a 0° C. solution of tert-butyl (2S)-2-[methoxy(methyl)carbamoyl]piperidine-1-carboxylate (525 mg, 1.93 mmol) in tetrahydrofuran (10 mL). The reaction mixture was then stirred at room temperature for 30 min. The reaction mixture was cooled to 0° C. and carefully quenched by dropwise addition of aqueous 5% KHSO₄ (10 mL). The mixture was then extracted with EtOAc (2×15 mL). The organic extracts were combined, washed with, sat. aqueous NaHCO₃ and saturated aqueous NaCl. The EtOAc was then dried over Na₂SO₄, filtered and concentrated. Yielded the title compound (392 mg, 1.84 mmol, 95% yield) as a colorless oil. HPLC-MS (API-ES) Exact mass for C₁₁H₁₉NO₃ [M+H]⁺ requires m/z 214.1443. found m/z 214.

### c) tert-butyl (2S)-2-[(R)-(2-bromoquinolin-4-yl)(hydroxy)methyl]piperidine-1-carboxylate and tert-butyl (2S)-2-[(S)-(2-bromoquinolin-4-yl)(hydroxy)methyl]piperidine-1-carboxylate

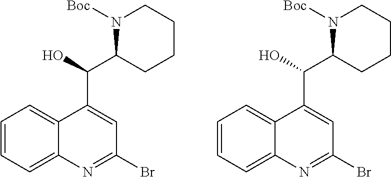 [SMILES: CC(C)(C)OC(=O)N1CCCC[C@H]1[C@H](O)c1cc(Br)nc2ccccc12 ; CC(C)(C)OC(=O)N1CCCC[C@H]1[C@@H](O)c1cc(Br)nc2ccccc12]

2,4-dibromoquinoline (0.45 g, 1.6 mmol) was dissolved in dry tetrahydrofuran. i-PrMgCl LiCl complex 1.3 M solution in tetrahydrofuran (1.5 mL, 1.9 mmol) was added slowly, drop wise, at 0° C. Reaction mixture was stirred at rt for 30 min. Aldehyde tert-butyl (2S)-2-formylpiperidine-1-carboxylate (vacqmg015) (1.6 mmol) dissolved in dry THF was added at room temperature and to the reaction mixture and stirred at rt for 4 h. (HPLC analysis indicated 55% conversion to product diastereoisomeric D1/D2 ratio ca 1.0:1.3). After the reaction was completed sat. NH₄Cl solution was added and the mixture was extracted with EtOAc (3×20 mL). The organic phase was separated and washed with brine (25 mL) The solution was dried over MgSO₄, filtered and then evaporated in vacuo. (crude 680 mg) The resultant colorless oil was chromatographed on silica gel eluting with ethyl acetate in heptane (1:3). Fractions 10-17 (10 mL) were collected and dried under vacuum to give tert-butyl (2S)-2-[(R)-(2-bromoquinolin-4-yl)(hydroxy)methyl]piperidine-1-carboxylate (139 mg, 21%) as a white solid HPLC-MS (API-ES) Exact mass for C₂₀H₂₆BrN₂O₃[M+H]⁺ requires m/z 421.1127. found m/z 421. Fractions 20-30 (10 ml) were collected and dried under vacuum to give tert-butyl (2S)-2-[(S)-(2-bromoquinolin-4-yl)(hydroxy)methyl]piperidine-1-carboxylate (162 mg, 25%) as a white solid. HPLC-MS (API-ES) Exact mass for C₂₀H₂₆BrN₂O₃[M+H]⁺ requires m/z 421.1127. found m/z 421.

### d) tert-butyl (2S)-2-[(R)-[2-(4-chlorophenyl)quinolin-4-yl (hydroxy)methyl]piperidine-1-carboxylate and tert-butyl (2S)-2-[(S)-[2-(4-chlorophenyl)quinolin-4-yl (hydroxy)methyl]piperidine-1-carboxylate

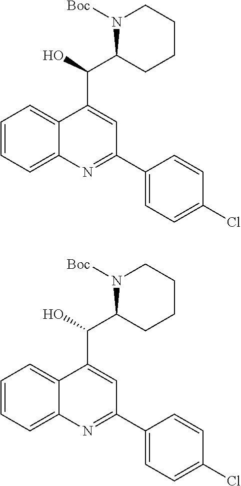 [SMILES: CC(C)(C)OC(=O)N1CCCC[C@H]1[C@H](O)c1cc(-c2ccc(Cl)cc2)nc2ccccc12 ; CC(C)(C)OC(=O)N1CCCC[C@H]1[C@@H](O)c1cc(-c2ccc(Cl)cc2)nc2ccccc12]

(2S)-2-[(R)-(2-bromoquinolin-4-yl)(hydroxy)methyl]piperidine-1-carboxylate (139 mg, 0.33 mmol) and boronic acid (62 mg, 0.4 mmol) were dissolved in DMF (1.7 mL) under N₂, PdCl₂(dppf) (2.7 mg, 0.03 mmol) and 2M K₂CO₃ (0.49 mL, 1.0 mmol) were added under nitrogen atmosphere and the reaction was heated at 90° C. over night. (HPLC-MS indicated 99% conversion). Purification by silica gel flash chromatography (EtOAc:heptane 1:3) and dried under vacuum. tert-butyl (2S)-2-[(R)-[2-(4-chlorophenyl)quinolin-4-yl (hydroxy)methyl]piperidine-1-carboxylate. (111 mg, 74%) as a white solid. HPLC-MS (API-ES) Exact mass for C₂₆H₂₉ClN₂O₃[M+H]⁺ requires m/z 453.1945. found m/z 453.

The same procedure was used starting from (2S)-2-[(S)-(2-bromoquinolin-4-yl)(hydroxy)methyl]piperidine-1-carboxylate to yield tert-butyl (2S)-2-[(S)-[2-(4-chlorophenyl)quinolin-4-yl (hydroxy)methyl]piperidine-1-carboxylate. Yielded tert-butyl (2S)-2-[(S)-[2-(4-chlorophenyl)quinolin-4-yl (hydroxy)methyl]piperidine-1-carboxylate. (65 mg, 37%) as a white solid. HPLC-MS (API-ES) Exact mass for C₂₆H₂₉ClN₂O₃[M+H]⁺ requires m/z 453.1945. found m/z 453.

### e) (R)-[2-(4-chlorophenyl)quinolin-4-yl](2S)-piperidin-2-ylmethanol (S20) and (S)-[2-(4-chlorophenyl)quinolin-4-yl](2S)-piperidin-2-ylmethanol (S22)

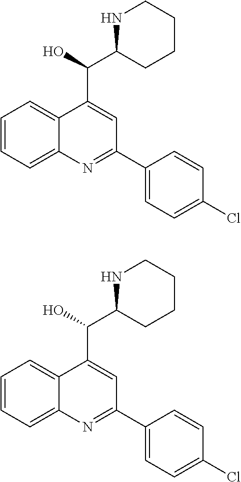 [SMILES: O[C@H](c1cc(-c2ccc(Cl)cc2)nc2ccccc12)[C@@H]1CCCCN1 ; O[C@@H](c1cc(-c2ccc(Cl)cc2)nc2ccccc12)[C@@H]1CCCCN1]

tert-Butyl (2S)-2-[(R)-[2-(4-chlorophenyl)quinolin-4-yl (hydroxy)methyl]piperidine-1-carboxylate (109 mg, 0.24 mmol) was dissolved in MeOH (1 mL) and cooled to 0° C. HCl 1M in Et₂O (1.45 mL, 1.45 mmol) was added and the solution was allowed to warm to room temperature over night. The formed precipitate was filtrated and dried under vacuum, to give crude product (68 mg with HPLC purity 85%). The crude material was dissolved in acetonitrile (2 mL) and ammonia 25% (1 mL) and purified by preparatory HPLC (MeCN: NH₃/NH₄HCO₃ (50 mM) 5 to 35%). Fraction was collected and dried under vacuum, to give S20 (42.5 mg, 50% yield) as a white solid. HPLC-MS (API-ES) Exact mass for C₂₁H₂₁ClN₂O [M+H]⁺ requires m/z 353.1421. found m/z 353. ¹H NMR (400 MHz, CHLOROFORM-d) δ8.19 (d, J=8.21 Hz, 1H), 8.16 (d, J=8.53 Hz, 2H), 8.10 (s, 1H), 7.94 (d, J=8.53 Hz, 1H), 7.66-7.77 (m, 1H), 7.51-7.56 (m, 1H), 7.49 (d, J=8.53 Hz, 2H), 5.45 (d, J=3.47 Hz, 1H), 3.06-3.21 (m, 2H), 2.74 (dt, J=2.69, 11.93 Hz, 1H), 1.72 (d, J=12.64 Hz, 1H), 1.57 (d, J=13.27 Hz, 1H), 1.29-1.46 (m, 2H), 1.06-1.22 (m, 2H) ¹³C NMR (101 MHz, CHLOROFORM-d) δ155.7, 148.3, 147.2, 138.1, 135.5, 130.6, 129.3, 128.9, 128.9, 126.3, 124.7, 122.7, 116.0, 72.5, 59.9, 46.9, 26.0, 25.0, 23.9

The same procedure was used starting from tert-butyl (2S)-2-[(S)-[2-(4-chlorophenyl)quinolin-4-yl (hydroxy)methyl]piperidine-1-carboxylate to yield (S)-[2-(4-chlorophenyl)quinolin-4-yl](2S)-piperidin-2-ylmethanol (S22). The crude material was dissolved in acetonitrile (2 mL) and ammonia 25% (1 ml) and purified by preparatory HPLC (MeCN: NH₃/NH₄HCO₃ (50 mM) 5 to 35%). Fraction was collected and dried under vacuum, to give S22 (28.5 mg, 54% yield) as a white solid. HPLC-MS (API-ES) Exact mass for C₂₁H₂₁ClN₂O [M+H]⁺ requires m/z 353.1421. found m/z 353. ¹H NMR (400 MHz, CHLOROFORM-d) δ8.18-8.22 (m, 1H), 8.14-8.18 (m, 2H), 8.02 (s, 1H), 7.95 (dd, J=0.63, 8.53 Hz, 1H), 7.74 (ddd, J=1.26, 7.03, 8.45 Hz, 1H), 7.55 (ddd, J=1.42, 6.95, 8.37 Hz, 1H), 7.48-7.52 (m, 2H), 5.26 (d, J=4.42 Hz, 1H), 3.08 (d, J=12.00 Hz, 1H), 2.90-2.98 (m, 1H), 2.56 (dt, J=2.69, 11.77 Hz, 1H), 1.76-1.85 (m, 1H), 1.50-1.64 (m, 3H), 1.42 (td, J=3.67, 12.24 Hz, 1H), 1.21-1.35 (m, 1H). ¹³C NMR (101 MHz, CHLOROFORM-d) δ155.6, 149.0, 148.4, 137.9, 135.6, 130.6, 129.5, 129.0, 128.8, 126.4, 125.0, 122.9, 115.7, 72.5, 61.0, 46.2, 29.4, 25.9, 24.2

### Example 8

### Stereoselective Synthesis of S21 and S23

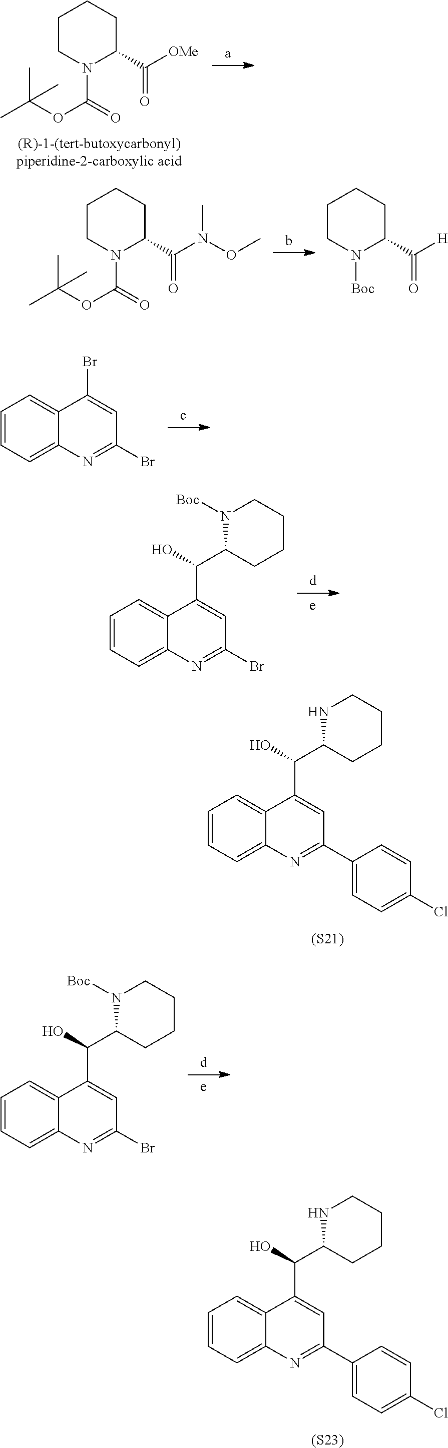

### a) tert-butyl (2R)-2-[methoxy(methyl)carbamoyl]piperidine-1-carboxylate

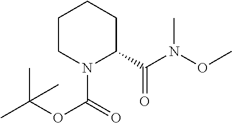 [SMILES: CON(C)C(=O)[C@H]1CCCCN1C(=O)OC(C)(C)C]

(R)-(D)-N-Boc-Pipecolic acid (0.5 g, 2.2 mmol) was dissolved in DMF (2.4 mL), diisopropylethylamine (2.2 mL, 4.4 mmol) was added followed by HATU (1.2 g, 3.3 mmol) at 22° C. The reaction mixture was stirred for 5 min. N,O-Dimethylhydroxylamine hydrochloride (0.3 g, 3.3 mmol) was added and reaction mixture was stirred at room temperature for 1 h The solution was diluted with EtOAc (20 mL) and poured into 1M HCl (20 ml). The organic phase was separated and washed with saturated aqueous sodium hydrogen carbonate (25 ml) and brine (25 ml) The solution was dried over MgSO₄, filtered and then evaporated in vacuum. The resultant colorless oil was chromatographed on silica gel eluting with 20% ethyl acetate in heptane. Fractions were collected, evaporated and dried under vacuum for 24 h. Yielded the title compound (546 mg, 92%) as a colourless oil. HPLC-MS (API-ES) Exact mass for C₁₃H₂₄N₂O₄ [M+H]⁺ requires m/z 273.1814. found m/z 273.

### b) tert-butyl (2R)-2-formylpiperidine-1-carboxylate

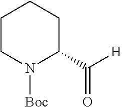 [SMILES: CC(C)(C)OC(=O)N1CCCC[C@@H]1C=O]

LiAlH₄ (1M in THF, 2.6 mL, 2.64 mmol) was added in portions to a 0° C. solution of tert-butyl (2R)-2-[methoxy(methyl)carbamoyl]piperidine-1-carboxylate (480 mg, 1.76 mmol) in tetrahydrofuran (10 mL). The reaction mixture was then stirred at room temperature for 30 min. The reaction mixture was cooled to 0° C. and carefully quenched by dropwise addition of aqueous 5% KHSO₄ (10 mL). The mixture was then extracted with EtOAc (2×15 mL). The organic extracts were combined, washed with, sat. aqueous NaHCO₃ and saturated aqueous NaCl. The EtOAc was then dried over Na₂SO₄, filtered and concentrated. Yielded the title compound (273 mg, 73% yield) as a colorless oil.

### c) tert-butyl (2R)-2-[(S)-(2-bromoquinolin-4-yl)(hydroxy)methyl]piperidine-1-carboxylate and tert-butyl (2R)-2-[(R)-(2-bromoquinolin-4-yl)(hydroxy)methyl]piperidine-1-carboxylate

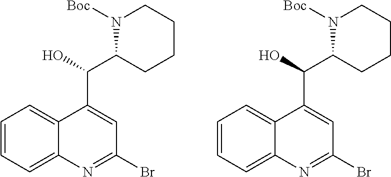 [SMILES: CC(C)(C)OC(=O)N1CCCC[C@@H]1[C@@H](O)c1cc(Br)nc2ccccc12 ; CC(C)(C)OC(=O)N1CCCC[C@@H]1[C@H](O)c1cc(Br)nc2ccccc12]

2,4-dibromoquinoline (0.37 g, 1.28 mmol) was dissolved in dry tetrahydrofuran. i-PrMgCl LiCl complex 1.3 M solution in tetrahydrofuran (1.3 mL, 1.66 mmol) was added slowly, drop wise, at 0° C. Reaction mixture was stirred at rt for 30 min. Tert-butyl (2R)-2-formylpiperidine-1-carboxylate (0.27 g, 1.28 mmol) dissolved in dry THF was added at room temperature and to the reaction mixture and stirred at rt for 4 h. (HPLC analysis indicated 99% conversion to product diastereoisomeric D3/D4 ratio ca 1.0:1.3). After the reaction was completed sat. NH₄Cl solution was added and the mixture was extracted with EtOAc (3×20 mL). The organic phase was separated and washed with brine (25 mL) The solution was dried over MgSO₄, filtered and then evaporated in vacuo. (crude yield 680 mg). The resultant colorless oil was chromatographed on silica gel eluting with ethyl acetate in heptane (1:3). Fractions 14-22 (10 mL) were collected and dried under vacuum to give tert-butyl (2R)-2-[(S)-(2-bromoquinolin-4-yl)(hydroxy)methyl]piperidine-1-carboxylate (156 mg, 29%) as a white solid HPLC-MS (API-ES) Exact mass for C₂₀H₂₆BrN₂O₃[M+H]⁺ requires m/z 421.1127. found m/z 421. Fractions 28-38 (10 mL) were collected and dried under vacuum to give tert-butyl (2R)-2-[(R)-(2-bromoquinolin-4-yl)(hydroxy)methyl]piperidine-1-carboxylate (150 mg, 28%) as a white solid. HPLC-MS (API-ES) Exact mass for C₂₀H₂₆BrN₂O₃[M+H]⁺ requires m/z 421.1127. found m/z 421.

### d) tert-butyl (2R)-2-[(S)-[2-(4-chlorophenyl)quinolin-4-yl (hydroxy)methyl]piperidine-1-carboxylate and tert-butyl (2R)-2-[(R)-[2-(4-chlorophenyl)quinolin-4-yl (hydroxy)methyl]piperidine-1-carboxylate

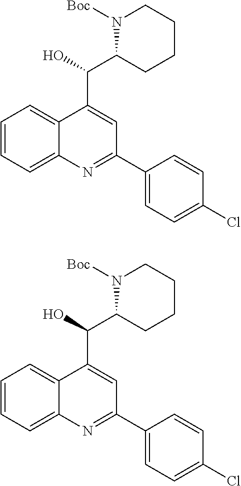 [SMILES: CC(C)(C)OC(=O)N1CCCC[C@@H]1[C@@H](O)c1cc(-c2ccc(Cl)cc2)nc2ccccc12 ; CC(C)(C)OC(=O)N1CCCC[C@@H]1[C@H](O)c1cc(-c2ccc(Cl)cc2)nc2ccccc12]

tert-butyl (2R)-2-[(S)-(2-bromoquinolin-4-yl)(hydroxy)methyl]piperidine-1-carboxylate (156 mg, 0.37 mmol) and 4-chlorophenylboronic acid (69 mg, 0.44 mmol) were dissolved in 2-methyltetrahydrofuran (1.9 mL) under N₂, PdCl₂(dppf) (3.0 mg, 0.04 mmol) and 2M K₂CO₃ (0.74 mL, 1.48 mmol) were added under nitrogen atmosphere and the reaction was heated at 90° C. over night. (HPLC-MS indicated 99% conversion). Purification by silica gel flash chromatography (EtOAc:heptane 1:3) and dried under vacuum. Yielded compound tert-butyl (2R)-2-[(S)-[2-(4-chlorophenyl)quinolin-4-yl (hydroxy)methyl]piperidine-1-carboxylate (141 mg, 84%) as a white solid. HPLC-MS (API-ES) Exact mass for C₂₆H₂₉ClN₂O₃[M+H]⁺ requires m/z 453.1945. found m/z 453.

The same procedure was used starting from (2R)-2-[(R)-(2-bromoquinolin-4-yl)(hydroxy)methyl]piperidine-1-carboxylate to yield tert-butyl (2R)-2-[(R)-[2-(4-chlorophenyl)quinolin-4-yl (hydroxy)methyl]piperidine-1-carboxylate. Purification by silica gel flash chromatography (EtOAc:heptane 1:3) and dried under vacuum. Yielded tert-butyl (2R)-2-[(R)-[2-(4-chlorophenyl)quinolin-4-yl (hydroxy)methyl]piperidine-1-carboxylate as a white solid. (150 mg, 93%) as a white solid. HPLC-MS (API-ES) Exact mass for C₂₆H₂₉ClN₂O₃ [M+H]⁺ requires m/z 453.1945. found m/z 453.

### e) (S)-[2-(4-chlorophenyl)quinolin-4-yl](2R)-piperidin-2-ylmethanol (S21) and (R)-[2-(4-chlorophenyl)quinolin-4-yl](2R)-piperidin-2-ylmethanol (S23)

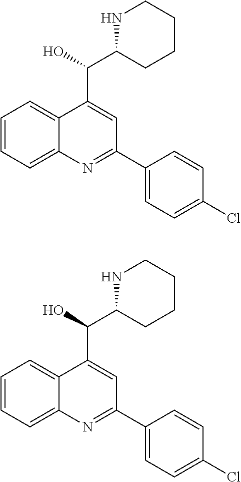 [SMILES: O[C@@H](c1cc(-c2ccc(Cl)cc2)nc2ccccc12)[C@H]1CCCCN1 ; O[C@H](c1cc(-c2ccc(Cl)cc2)nc2ccccc12)[C@H]1CCCCN1]

tert-butyl (2R)-2-[(S)-[2-(4-chlorophenyl)quinolin-4-yl (hydroxy)methyl]piperidine-1-carboxylate (156 mg, 0.34 mmol) was dissolved in MeOH (1.7 mL) and cooled to 0° C. HCl 1M in Et₂O (1.7 mL, 1.72 mmol) was added and the solution was allowed to warm to room temperature over night. The formed precipitate was filtrated and dried under vacuum, to give crude product (68 mg with HPLC purity 85%). The crude material was dissolved in acetonitrile (2 mL) and ammonia 25% (1 mL) and purified by preparatory HPLC (MeCN: NH₃/NH₄HCO₃ (50 mM) 5 to 35%). Fraction was collected and dried under vacuum, to give (S)-[2-(4-chlorophenyl)quinolin-4-yl](2R)-piperidin-2-ylmethanol (S21) (82 mg, 67% yield) as a white solid. HPLC-MS (API-ES) Exact mass for C₂₁H₂₁ClN₂O [M+H]⁺ requires m/z 353.1421. found m/z 353. ¹H NMR (400 MHz, CHLOROFORM-d) δ 8.19 (dd, J=1.11, 8.69 Hz, 1H), 8.13-8.17 (m, 2H), 8.10 (s, 1H), 7.93 (dd, J=0.63, 8.53 Hz, 1H), 7.72 (ddd, J=1.26, 7.03, 8.45 Hz, 1H), 7.50-7.54 (m, 1H), 7.46-7.50 (m, 2H), 5.44 (d, J=3.47 Hz, 1H), 3.15 (td, J=1.86, 11.77 Hz, 1H), 3.10 (td, J=3.00, 11.37 Hz, 1H), 2.73 (dt, J=2.84, 12.00 Hz, 1H), 1.67-1.76 (m, 1H), 1.53-1.61 (m, 1H), 1.29-1.44 (m, 2H), 1.06-1.21 (m, 2H). ¹³C NMR (101 MHz, CHLOROFORM-d) δ 155.7, 148.3, 147.3, 138.1, 135.5, 130.6, 129.3, 128.9, 128.9, 126.3, 124.7, 122.7, 116.0, 72.5, 59.9, 46.9, 26.0, 25.0, 23.9

The same procedure was used starting from tert-butyl (2R)-2-[(R)-[2-(4-chlorophenyl)quinolin-4-yl (hydroxy)methyl]piperidine-1-carboxylate to yield (R)-[2-(4-chlorophenyl)quinolin-4-yl](2R)-piperidin-2-ylmethanol (S23). The crude material was dissolved in acetonitrile (2 mL) and ammonia 25% (1 mL) and purified by preparatory HPLC (MeCN:NH₃/NH₄HCO₃ (50 mM) 5 to 35%). Fraction was collected and dried under vacuum, to give title compound (55 mg, 48% yield) as a white solid. HPLC-MS (API-ES) Exact mass for C₂₁H₂₁ClN₂O [M+H]⁺ requires m/z 353.1421. found m/z 353. ¹H NMR (400 MHz, CHLOROFORM-d) δ 8.19 (dd, J=0.95, 8.53 Hz, 1H), 8.10-8.16 (m, 2H), 7.99 (s, 1H), 7.93 (dd, J=0.95, 8.53 Hz, 1H), 7.73 (ddd, J=1.26, 6.79, 8.37 Hz, 1H), 7.50-7.55 (m, 1H), 7.46-7.50 (m, 2H), 5.23 (d, J=4.74 Hz, 1H), 3.06 (d, J=11.69 Hz, 1H), 2.91 (td, J=5.17, 8.29 Hz, 1H), 2.56 (dt, J=2.84, 11.85 Hz, 1H), 1.77 (td, J=1.58, 12.95 Hz, 1H), 1.48-1.62 (m, 3H), 1.34-1.48 (m, 1H), 1.19-1.33 (m, 1H). ¹³C NMR (101 MHz, CHLOROFORM-d) δ 155.6, 149.1, 148.4, 137.9, 135.6, 130.6, 129.5, 129.0, 128.8, 126.4, 125.0, 122.9, 115.8, 72.5, 61.1, 46.3, 29.3, 25.8, 24.2

Persons skilled in the art may find alternative routes of synthesis for the disclosed substances. The non-limiting examples presented above is in no way intended to limit the scope of the invention. Preparation of S10, S20, S21, S22 and S23 can also be achieved as in Example 7 and 8 using N-Boc-2-piperidinyl aldehyde, or optionally protected with other protected groups known to those skilled in the art.

To those skilled in the art, preparation of S10, S20, S21, S22 and S23 can also be achieved as in Example 7 and 8 using the corresponding 2-piperidinyl ester, Weinreb amide or other activated carboxylic acid derivative followed by reduction of the resulting ketone.

### Chiral Chromatography

Stereoselective isolation of S20, S21, S22 and S23 can also be achieved using preparative chiral chromatography. Without intending to limit the scope of the invention, in one example, the following general methods were used to purify up to 50 mg of S20, S21, S22 and S23, respectively:

### Analytical System (Achiral Method): LC05

Columns: Kromasil 100-5SIL, 4.6×250 mm

Mobile phase A: Heptane+0.1% diethylamine (DEA), Mobile phase B: Ethanol+0.1% DEA

Isocratic method: Mobile phase A/B 80/20+DEA

Temperature: 35° C., inj. volume: 25 μL, Flow rate: 1 mL/min, UV: 265 nm

### Analytical System (Chiral Method): LC05

Columns: ChiralPak AD-H, 4.6×250 mm, 5 m

Mobile phase A: Heptane+0.1% DEA, Mobile phase B: Ethanol+0.1% DEA

Isocratic method: Mobile phase A/B 70/30+DEA

Temperature: 35° C., inj. volume: 5 μL, Flow rate: 1 mL/min, UV: 265 nm

### Analytical System (Chiral Method): LC05

Columns: ChiralPak OD-H, 4.6×250 mm, 5 μm

Mobile phase A: Heptane+0.1% DEA, Mobile phase B: Ethanol+0.1% DEA

Isocratic method: Mobile phase A/B 90/10+DEA

Temperature: 22° C., inj. volume: 5 μL, Flow rate: 1 mL/min, UV: 265 nm

### Preparative Achiral Method (Knauer): LC07

Columns: Kromasil Silica 10 mm (100A) 50×190 mm

Mobile phase A: Heptane, Mobile phase B: Ethanol+0.2% DEA

Isocratic system: Heptane/Ethanol+0.2% DEA 50/50

Temp: room temp, Inj vol: 0.5-10 mL, Flow rate: 100 mL/min, UV: 265 nm

Semi-Prep chiral method: LC02
Samples: 14S0090 och 14S0091
Column: OD-H, 4.6 × 250 mm, 5 μm
Mobile phase A: Heptane
Mobile phase B: Ethanol + 0.2% DEA
Gradient:

| t (min) | % B | mL/min |
|---|---|---|
| 0 | 5 | 2 |
| 2 | 5 | 2 |
| 3 | 5 | 20 |
| 10 | 5 | 20 |
| 11.1 | 5 | 2 |

UV: 265 nm
Inj. vol: 0.5 mL
Temp: 22° C.

Semi-Prep chiral method: LC02
Samples: 14S0074 och 14S0075
Column: AD-H, 4.6 × 250 mm, 5 μm
Mobile phase A: Heptane
Mobile phase B: Ethanol + 0.2% DEA
Gradient:

| t (min) | % B | mL/min |
|---|---|---|
| 0 | 60 | 2 |
| 2 | 60 | 2 |
| 18 | 60 | 15 |
| 18.5 | 60 | 2 |
| 19 | 60 | 2 |

UV: 265 nm
Inj. vol: 1 mL
Temp: 35° C.

Stereoselective isolation of S20, S21, S22 and S23 can also be achieved using chiral crystallization methods known to those skilled in the art.

### Example 9

### Synthesis of the mixture of 5-(4-((R)-hydroxy((S)-piperidin-2-yl)methyl)quinolin-2-yl)-2-methylbenzonitrile and 5-(4-((S)-hydroxy((R)-piperidin-2-yl)methyl)quinolin-2-yl)-2-methylbenzonitrile (S24). General Method C

### Mixture of tert-butyl (R)-2-((S)-(2-bromoquinolin-4-yl) (hydroxy)methyl)piperidine-1-carboxylate and tert-butyl (S)-2-((R)-(2-bromoquinolin-4-yl) (hydroxy)methyl)piperidine-1-carboxylate (Intermediate 27) and mixture of tert-butyl (R)-2-((R)-(2-bromoquinolin-4-yl) (hydroxy)methyl)piperidine-1-carboxylate tert-butyl (S)-2-((S)-(2-bromoquinolin-4-yl) (hydroxy)methyl)piperidine-1-carboxylate (Intermediate 28)

2,4-Dibromoquinoline (502 mg, 1.76 mmol) was dissolved in dry THF (4.5 mL). i-PrMgCl*LiCl (1.47 mL, 1.3 M in THF, 1.91 mmol) was added dropwise at room temperature under N₂ atmosphere, followed by addition of a solution of tert-butyl 2-formylpiperidine-1-carboxylate (486 mg, 2.28 mmol). The reaction mixture was stirred at room temperature for 24 h. NH₄Cl (aq., sat) was added, and the mixture was extracted four times with EtOAc. The combined organic solutions were washed twice with Brine and dried (MgSO₄). Evaporation of the solvent gave the crude product (1.02 g), which was purified by flash chromatography (gradient of EtOAc/i-hexane 10:90 to 30:70) to give the intermediates 27 and 28.

Intermediate 27. Fractions 34-60, 205 mg, 28%, white solid. MS (ESI⁺) m/z 421 [M+H]⁺.

Intermediate 28: Fractions 70-90, 212 mg, 29%, white solid. MS (ESI⁺) m/z 421 [M+H]⁺.

The relative stereochemistry of the intermediates 27 and 28, respectively, were determined by comparison to the relative retention order of the same intermediates in the synthesis of compounds S20-S23.

A solution of intermediate 27 (21 mg, 0.050 mmol), 3-cyano-4-methylphenylboronic acid (10 mg, 0.062 mmol), Pd(dppf)Cl₂*CH₂Cl₂ (2.7 mg, 0.003 mmol) and DIPEA (40 μL, 0.230 mmol) in aqueous dioxane (0.55 mL, 10% H₂O) was heated at 80° C. under N₂ atmosphere for 15 h. The reaction mixture was diluted with MeCN, filtrated and purified by preparative reverse-phase HPLC using basic conditions. The pure fractions were combined and the solvent was removed under reduced pressure giving a mixture of tert-butyl (S)-2-((R)-(2-(3-cyano-4-methylphenyl)quinolin-4-yl)(hydroxy)methyl)piperidine-1-carboxylate and tert-butyl (R)-2-((S)-(2-(3-cyano-4-methylphenyl)quinolin-4-yl)(hydroxy)methyl)piperidine-1-carboxylate (8.5 mg). MS (ESI⁺) m/z 458 [M+H]⁺.

The mixture of tert-butyl (S)-2-((R)-(2-(3-cyano-4-methylphenyl)quinolin-4-yl)(hydroxy)methyl)piperidine-1-carboxylate and tert-butyl (R)-2-((S)-(2-(3-cyano-4-methylphenyl)quinolin-4-yl)(hydroxy)methyl)piperidine-1-carboxylate (8.5 mg) was dissolved in CH₂Cl₂ (0.5 mL). 1M HCl in Et₂O (1.0 mL, 1.0 mmol) was added and the reaction mixture was stirred at room temperature for 24 h. The solvent was removed by evaporation, giving the mixture of 5-(4-((R)-hydroxy((S)-piperidin-2-yl)methyl)quinolin-2-yl)-2-methylbenzonitrile and 5-(4-((S)-hydroxy((R)-piperidin-2-yl)methyl)quinolin-2-yl)-2-methylbenzonitrile as HCl salt (white solid, 8.2 mg, 42% yield over two steps). ¹H NMR (400 MHz, Methanol-d₄) δ ppm 8.65 (d, J=7.9 Hz, 1H) 8.50 (s, 2H) 8.44 (d, J=8.5 Hz, 1H) 8.33 (d, J=7.9 Hz, 1H) 8.16-8.26 (m, 1H) 7.99-8.11 (m, 1H) 7.81 (d, J=7.9 Hz, 1H) 6.11 (s, 1H) 3.73 (d, J=11.4 Hz, 1H) 3.47 (d, J=11.1 Hz, 1H) 3.19 (t, J=12.3 Hz, 1H) 2.72 (s, 3H) 1.58-1.97 (m, 4H) 1.23-1.49 (m, 2H). MS (ESI⁺) m/z 358 [M+H]⁺.

The compounds S25-S29 were prepared according to General Method C, illustrated in Example 9 and Table 2.

TABLE 2
Synthetic details and analytical data for compounds S25-S29.

| | | | MS | React. | Yield |
|---|---|---|---|---|---|
| | Start | | (ESI⁺) | time at | over 2 |
| Compound | mtrl | ¹H NMR data | m/z | 80° C. | steps |
| S25 | IM27 | ¹H NMR (400 MHz, Methanol-d₄) δ ppm 8.55 | 446 | 20 h | 58% |
| | | (d, J = 8.5 Hz, 1 H) 8.48 (s, 1 H) 8.40 (d, J = 8.5 | [M + H]⁺ | | |
| | | Hz, 1 H) 8.25 (d, J = 8.2 Hz, 2 H) 8.12-8.19 | | | |
| | | (m, 1 H) 7.97-8.04 (m, 1 H) 7.71 (d, J = 8.2 | | | |
| | | Hz, 2 H) 6.06 (d, J = 2.5 Hz, 1 H) 3.67-3.78 | | | |
| | | (m, 1 H) 3.50-3.58 (m, 2 H) 3.44-3.50 (m, 1 | | | |
| | | H) 3.24-3.29 (m, 2 H) 3.14-3.24 (m, 1 H) | | | |
| | | 1.53-1.95 (m, 8 H) 1.25-1.47 (m, 2 H) 1.03 | | | |
| | | (t, J = 7.4 Hz, 3 H) 0.78 (t, J = 7.4 Hz, 3 H) | | | |
| S26* | IM27 | ¹H NMR (400 MHz, Methanol-d₄) δ ppm 8.39 | 387 | 6 h, | 26% |
| | | (d, J = 8.2 Hz, 2 H) 8.31 (d, J = 0.6 Hz, 1 H) 8.24 | [M + H]⁺ | followed | |
| | | (dd, J = 8.5, 0.6 Hz, 1 H) 8.16 (d, J = 8.5 Hz, 1 H) | | by 3 | |
| | | 7.84-7.93 (m, 3 H) 7.74 (ddd, J = 8.5, 7.0, 1.3 | | days at | |
| | | Hz, 1 H) 5.85 (d, J = 2.5 Hz, 1 H) 3.66 (dt, | | 65° C. | |
| | | J = 12.0, 2.7 Hz, 1 H) 3.41-3.50 (m, 1 H) 3.16 | | | |
| | | (td, J = 12.6, 3.3 Hz, 1 H) 1.77-1.89 (m, 2 H) | | | |
| | | 1.66-1.74 (m, 1 H) 1.26-1.41 (m, 3 H) | | | |
| S27 | IM27 | ¹H NMR (400 MHz, Methanol-d₄) δ ppm 9.48 | 388 | 6 h, | 42% |
| | | (s, 1 H) 8.82 (dd, J = 8.2, 1.9 Hz, 1 H) 8.42-8.52 | [M + H]⁺ | followed | |
| | | (m, 2 H) 8.37 (d, J = 8.9 Hz, 1 H) 8.04-8.16 (m, | | by 3 | |
| | | 2 H) 7.94 (t, J = 7.4 Hz, 1 H) 6.03 (br. s., 1 H) | | days at | |
| | | 3.71 (d, J = 12.0 Hz, 1 H) 3.42-3.52 (m, 1 H) | | 65° C. | |
| | | 3.18 (td, J = 12.9, 2.7 Hz, 1 H) 1.67-1.92 (m, 4 | | | |
| | | H) 1.31-1.44 (m, 2 H) | | | |
| S28 | IM28 | ¹H NMR (400 MHz, Methanol-d₄) δ ppm 8.54 | 387 | 6 h, | 33% |
| | | (d, J = 8.8 Hz, 1 H) 8.52 (s, 1 H) 8.42 (dd, | [M + H]⁺ | followed | |
| | | J = 8.5, 0.6 Hz, 1 H) 8.39 (d, J = 8.2 Hz, 2 H) | | by 3 | |
| | | 8.16 (ddd, J = 8.5, 7.1, 1.1 Hz, 1 H) 8.04 (d, | | days at | |
| | | J = 8.2 Hz, 2 H) 7.99 (ddd, J = 8.5, 7.1, 1.1 Hz, 1 | | 65° C. | |
| | | H) 5.73 (d, J = 6.3 Hz, 1 H) 3.66 (ddd, J = 11.8, | | | |
| | | 6.4, 3.0 Hz, 1 H) 3.37-3.45 (m, 1 H) 2.97 (td, | | | |
| | | J = 13.0, 3.0 Hz, 1 H) 1.61-1.95 (m, 5 H) 1.44- | | | |
| | | 1.59 (m, 1 H) | | | |
| S29 | IM28 | ¹H NMR (400 MHz, Methanol-d₄) δ ppm 9.52 | 388 | 6 h, | 57% |
| | | (d, J = 2.2 Hz, 1 H) 8.87 (ddd, J = 8.2, 2.2, 0.6 Hz, | [M + H]⁺ | followed | |
| | | 1 H) 8.47 (s, 1 H) 8.45 (d, J = 7.9 Hz, 1 H) 8.34- | | by 3 | |
| | | 8.38 (m, 1 H) 8.11 (dd, J = 8.4, 0.8 Hz, 1 H) 8.06 | | days at | |
| | | (ddd, J = 8.5, 7.0, 1.3 Hz, 1 H) 7.90 (ddd, J = 8.5, | | 65° C. | |
| | | 7.0, 1.3 Hz, 1 H) 5.67 (d, J = 6.6 Hz, 1 H) 3.64 | | | |
| | | (ddd, J = 11.8, 6.6, 3.2 Hz, 1 H) 3.37-3.46 (m, 1 | | | |
| | | H) 2.97 (td, J = 13.0, 3.2 Hz, 1 H) 1.61-1.97 (m, | | | |
| | | 5 H) 1.41-1.60 (m, 1 H) | | | |

*For the preparation of compound S26, the N-^{t}BOC protected intermediate and S26, respectively, were purified by preparative reverse-phase HPLC using acidic conditions giving the trifluoroacetic acid salt of S26 as a white solid.

### Example 10

### Stereoselective Synthesis of Vacquinol-1 RS (S20)

A stereoselective synthesis of Vacquinol-1RS was designed based on a modification of León (León, B., et al (2013). Organic Letters, 15(6), 1234-7), according to the following Scheme.

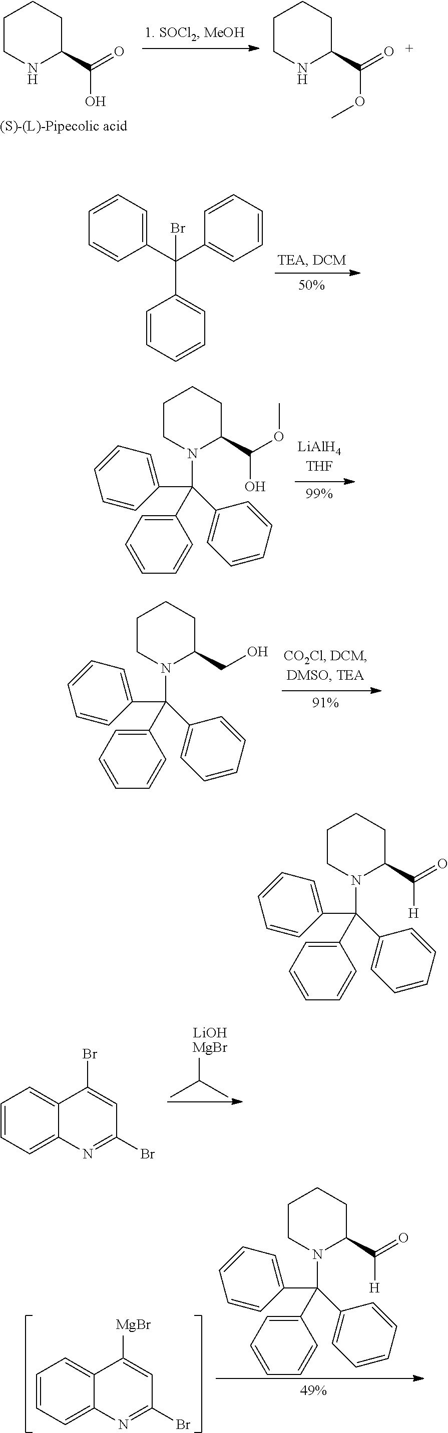

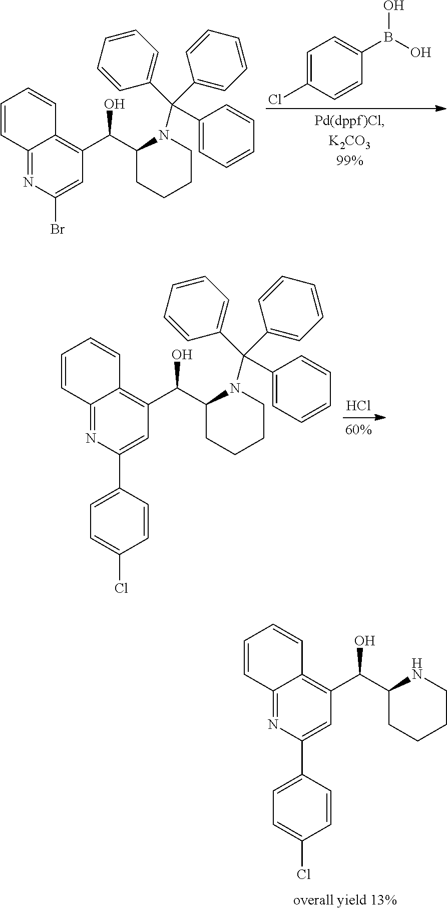

Briefly, tritylation of methylated (S)-L-Pipecolic acid afforded the possibility to generate a chiral piperidine carbaldehyde material suitable for face-selective addition by the Grignard reagent generated from 2,4-dibromoquinoline. The single isolated R,S isomer was then subject to Suzuki coupling of the appropriate 4-chlorophenylboronic acid, which after concomitant deprotection of the trityl group yields the desired (R)-[2-(4-chlorophenyl)quinolin-4-yl](2S)-piperidin-2-ylmethanol.

### Methyl (2S)-piperidine-2-carboxylate

(S)-(L)-Pipecolic acid (1.5 g, 11.61 mmol) was added to methanol (11.6 mL) under N₂. To this solution thionyl chloride (1.69 mL, 23.23 mmol) was slowly added at −10° C. The reaction mixture was allowed to warm to rt and was stirred for 18 hours. Reaction mixture was evaporated and co-evaporated with toluene and dried under vacuum. The crude was used in next step.

### Methyl (2S)-1-(triphenylmethyl)piperidine-2-carboxylate

Methyl (2S)-piperidine-2-carboxylate (1.66 g, 11.59 mmol) was dissolved in CH₂Cl₂ (13 mL), then Et₃N (4.85 mL, 34.78 mmol) was added. To this solution was added trityl bromide (3.75 g, 11.59 mmol) reaction mixture was stirred for 18 h at rt. The reaction was hydrolyzed with NH₄Cl/28% NH₃ (6 mL, 2:1). The solution was partitioned between Et₂O (20 mL) and H₂O (20 mL). The layers were separated and the aqueous layer was extracted with Et₂O (3×30 mL). The combined organic layers were dried with MgSO₄, filtered, and concentrated in vacuo. The residue was purified by flash chromatography (1:2:97, Et₃N:EtOAc:Heptane) to title compound (2.21 g, 50%) as a white foam. HPLC-MS (API-ES) Exact mass for C₂₆H₂₇NO₂ [M+H]⁺ requires m/z 386.2120. found m/z.

### [(2S)-1-(triphenylmethyl)piperidin-2-yl]methanol

To an oven dried 3-neck flask (100 mL) equipped with a stir bar (N2) and condenser was added THF (10 mL). To this solution was added LiAlH4 (0.47 g, 12.6 mmol) and was allowed to stir to form a suspension. To this suspension was added Methyl (2S)-1-(triphenylmethyl)piperidine-2-carboxylate (2.2 g, 8.42 mmol). The reaction solution was allowed to stir for 3 h at rt. (Became thick suspension after 30 min and 10 ml THF was added). The reaction mixture was then cautiously quenched with NaOH (1 mL, 1 M), and H₂O (2 mL). The solution became visibly thicker and more difficult to stir. MgSO₄ was then added and the solution was passed through a pad of celite with 300 mL of dichloromethane. This was then concentrated in vacuo. The residue was purified by flash chromatography (1:1:98, Et₃N:MeOH:CH₂Cl₂) to title compound (1.7 g, 99%) as a white foam. HPLC-MS (API-ES) Exact mass for C₂₅H₂₇NO [M+H]⁺ requires m/z 358.2170. found m/z 116. [M−Tr+H]⁺

### (2S)-1-(triphenylmethyl)piperidine-2-carbaldehyde

To an oven dried flask (100 mL) equipped with a stir bar (N₂) was added CH₂Cl₂ (5.7 mL) and was then taken to −78° C. To this solution was slowly added (COCl)₂ (0.61 mL, 7.13 mmol). Next a solution of DMSO (0.84 mL, 11.9 mmol) in CH₂Cl₂ (3.3 mL) was added dropwise. This was allowed to stir for 10 min and a solution of [(2S)-1-(triphenylmethyl)piperidin-2-yl]methanol (1.7 g, 4.76 mmol) in CH₂Cl₂ (4.28 mL) was then added. The suspension was allowed to stir for 1.5 h and then Et₃N (2.65 mL, 19.0 mmol) was added and allowed to stir for an additional 1.5 h. The −78° C. bath was then removed and NH₄Cl/28% NH₃ (20 mL, 2:1) was added and the solution was partitioned between CH₂Cl₂ (30 mL) and H2O (30 mL). The layers were separated and the aqueous layer was extracted with CH₂Cl₂ (3×70 mL). The combined organic layers were dried with MgSO₄, filtered, and concentrated in vacuo. The residue was purified by flash chromatography (1:9:90, Et₃N:EtOAc:Heptane) to afford title compound (1.54 g, 91%) as a white solid. HPLC-MS (API-ES) Exact mass for C₂₅H₂₅NO [M+H]⁺ requires m/z 355.1936. found m/z 114 [M-Tr+H]⁺

### (S)-(2-bromoquinolin-4-yl) [(2R)-1-(triphenylmethyl)piperidin-2-yl]methanol

2,4-dibromoquinoline (1.61 g, 5.63 mmol) was dissolved in dry tetrahydrofuran. i-PrMgCl LiCl complex 1.3 M solution in tetrahydrofuran (6.6 mL, 8.66 mmol) was added slowly, drop wise, at 0° C. Reaction mixture was stirred at rt for 30 min. (2R)-1-(triphenylmethyl)piperidine-2-carbaldehyde (1.54 g, 4.33 mmol) dissolved in dry THF was added at room temperature and to the reaction mixture and stirred at rt for 4 h. After the reaction was completed NH₄Cl (sat.)/NH₃(28%) solution was added and the mixture was extracted with DCM (3×20 mL). The organic phase was separated and washed with brine (25 mL) The solution was dried over MgSO₄, filtered and then evaporated in vacuo. The resultant oil was chromatographed on silica gel eluting with TEA:ethyl acetate:heptane (1:10:90). Fractions were collected and dried under vacuum to give title compound (1.188 mg, 49%) as a white solid. Exact mass for C₃₄H₃₂BrN₂O [M+H]⁺ requires m/z 563.1698, HPLC-MS (API-ES) (ACE C8 10-90% MeCN 1.5 min (0.1% TFA pH 2) (API-ES) C₁₅H₁₈ClN₂O [M+H]f requires m/z 321.0602 found m/z 321, (Trityl-group is removed under acidic conditions).

### (R)-[2-(4-chlorophenyl)quinolin-4-yl][(2S)-1-(triphenylmethyl)piperidin-2-yl]methanol

(R)-(2-bromoquinolin-4-yl)[(2S)-1-(triphenylmethyl)piperidin-2-yl]methanol (613 mg, 1.1 mmol) and 4-chlorophenylboronic acid (180 mg, 1.1 mmol) were dissolved in 2-MeTHF (5.5 mL) under N₂, PdCl₂(dppf) (71 mg, 0.09 mmol) and 2M K₂CO₃ (2.2 mL, 4.4 mmol) were added under nitrogen atmosphere and the reaction was heated at 90° C. over night. Filtrated and dried under vacuum to give title compound (650 mg, 99%). HPLC-MS (API-ES) Exact mass for C₄₀H₃₅ClN₂O [M+H]⁺ requires m/z 594.2437. found m/z 353 (Trityl group is removed under acidic conditions).

### (R)-[2-(4-chlorophenyl)quinolin-4-yl](2S)-piperidin-2-ylmethanol

(R)-[2-(4-chlorophenyl)quinolin-4-yl][(2S)-1-(triphenylmethyl)piperidin-2-yl]methanol (810 mg, 1.36 mmol) was dissolved in Et₂O (46 mL) followed by addition of 5M HCl (5.7 mL). After stirring at room temperature for 4 h. The solution was partitioned between Et₂O (60 mL) and H₂O (60 mL). The aqueous layer was extracted with Et₂O (3×50 mL). The aqueous layer was then basified with 6M NaOH, and then was extracted with CH₂Cl₂ (50 mL). The CH₂Cl₂ layer was dried with MgSO₄, filtered, and concentrated in vacuo. Purification by flash chromatography (Et₃N:MeOH:CH₂Cl₂, 1:1:98) yielded (264 mg, 55%) (R)-[2-(4-chlorophenyl)quinolin-4-yl](2S)-piperidin-2-ylmethanol. The material was purified by preparatory HPLC (MeCN:TFA 0.1% in H₂O 5 to 90%). Fraction was collected and concentrated under vacuum, pH was adjusted to pH 13 and the water phase was extracted with CH₂Cl₂ 3×50 ml. CH₂Cl₂ phase was Na₂SO₄ dried and evaporated, to give title compound (280 mg, 0.80 mmol, 60% yield) as a white solid. HPLC-MS (API-ES) Exact mass for C21H21ClN2O [M+H]+ requires m/z 353.1421. found m/z 353.

### Example 11

### Pharmacokinetic Evaluation of Vacquinol-1 Stereoisomers

Due to the superior in vitro efficacy of Vacquinol-1RS and Vacquinol-1SR over the previously studied isomeric mixture (Vacquinol-1 (racemic), NSC13316), it was desirable to investigate in vivo pharmacokinetic parameters of the individual isomers (RS and SR) of Vacquinol-1 versus the stereoisomeric mixture of all four isomers (RS/SR/RR/SS, NSC13316) by non-compartmental analysis.

The pharmacokinetics of Vacquinol-1 (racemic), Vacquinol-1RS and Vacquinol-1SR, were determined in NMRI (SR/RS) or BALB/c (Vrac) mice following single intravenous (i.v.) or per oral (p.o) administration of 2 or 20 mg/kg Vacquinol-1, respectively. Blood and brain samples were taken from animals at the following nominal time points: 15, 30, and 60 minutes, and 2, 4, 6, 8, 24, 48, 72 and 144 hours after dosing (n=3/time-point). Bioanalytical quantification of Vacquinol-1 was analysed in plasma and brain samples by a UPLC-MS/MS.

Pharmacokinetics were calculated by non-compartmental analysis (NCA) from composite (mean) profiles. Nominal sampling times and dose levels have been used for the NCA calculations.

TABLE 3
Summarised pharmacokinetic parameters after administration of 2 (i.v.) or
20 (p.o.) mg/kg racemic Vacquinol-1 (Vrac), Vacquinol-1_{RS} (RS) and Vacquinol-1_{SR} (SR) to
mice.

| | | | Tissue | | | | | | | | | |
|---|---|---|---|---|---|---|---|---|---|---|---|---|
| | | | Brain | | | | | Plasma | | | | |
| Dose | | | Cₘₐₓ | tₘₐₓ | AUCₗₐₛₜ | tₗₐₛₜ | t_{1/2} | Cₘₐₓ | tₘₐₓ | AUCₗₐₛₜ | tₗₐₛₜ | t_{1/2} |
| (mg/kg) | Isomer | Route | (ng/mL) | (h) | (h * ng/mL) | (h) | (hr) | (ng/mL) | (h) | (h * ng/mL) | (h) | (hr) |
| 2 | Vrac | i.v. | 514 | — | 20368 | 144 | 63 | 467 | — | 64500 | 144 | 52 |
| 2 | RS | i.v. | 1970 | 0.25 | 52700 | 144 | 67 | 775 | 4.0 | 62000 | 144 | 84 |
| 2 | SR | i.v. | 777 | 1.0 | 9050 | 72 | — | 455 | 0.50 | 14100 | 72 | — |
| 20 | Vrac | p.o. | 1860 | 48 | 166800 | 144 | — | 3280 | 24 | 291700 | 144 | — |
| 20 | RS | p.o. | 4840 | 8.0 | 400000 | 144 | — | 2210 | 4.0 | 246000 | 144 | — |
| 20 | SR | p.o. | 1490 | 6.0 | 157000 | 144 | — | 2050 | 8.0 | 183000 | 144 | — |

All animals dosed with Vrac, RS and SR were systemically exposed to the test compound. The plasma and brain concentrations were detectable and analysed until 144 h, with the exception of isomer SR at 2 mg/kg, i.v. administration, detectable until 72 h. It was observed that the Cₘₐₓ in brain tissue was considerably higher for RS compared to SR or Vrac, both after i.v. and p.o administration. The relative brain/plasma exposure ratio (AUCₗₐₛₜ(brain)/AUCₗₐₛₜ(plasma)) was 1.6 for RS after oral dosing, whilst only 0.9 for SR and 0.6 for Vrac. Cmax exposures ratios (Cₘₐₓ(brain)/Cₘₐₓ(plasma) were consistent with this finding, yielding 2.2 for RS, 0.7 for SR and 0.6 for Vrac.

Multi-phase elimination curves of all dosed compounds could be seen after i.v. administration with elimination half-lives was between 52 to 96 h after i.v. or p.o. administration. See, FIGS. 6A and 6B. This data shows the superior brain exposure of Vacquinol-1RS versus the corresponding SR isomer or the previously described stereoisomeric mixture (Vacquinol-1, NSC13316), whilst minimizing systemic exposure of the compound.

### Example 12

### Comparison with Mefloquine

Vacquinol-1 RS (S20) and mefloquine were evaluated for their relative cytotoxicities against glioblastoma cells (U3013) and human fibroblasts using standard methods. The comparative IC95 values for cell death are IC95 (Vacquinol-1RS)=8.9 μM and IC95 (mefloquine)=25.2 μM. See, FIGS. 7A and 7B. Said IC95 values were determined according to the methods described in the section below entitled, “In vitro cancer cell and CSC viability assay”.

### Example 13

### Pharmacological Assays

The ability of the aforementioned compounds S1-S23 to selectively modulate cancer cells, such as glioma cancer, are determined using assays known in the art or by novel in vitro and in vivo assays. The bioactivity of compounds described herein was tested according to the following assays.

### In Vitro Phenotypic Selectivity Screening Assay

In order to identify pathways susceptible for targeted treatment of glioma cancer cells or glioma stem cells (GSCs), a phenotypic screen was performed to identify compounds active on glioma cancer cells or GSCs without affecting embryonic stem cells or human fibroblasts. Adherent GSC cultures were independently generated from two cases of glioblastoma multiforme according to Pollard et al., (Pollard S M, (2009) Cell Stem Cell, 4, 568-580) designated U3013MG and U3047MG and were screened, rescreened and confirmed using 1364 compounds of the NIH diversity set II for phenotypic changes observed following phalloidin staining. 237 compounds showed effects after two days and 63 compounds showed selective effects on GSCs. The 63 compounds were confirmed active on U3013MG and U3047MG GSCs as well as on seven other established GSC culture, U3024MG, U3017MG, U3031MG, U3037MG, U3086MG, U3054MG, U3065MG. Microarray analysis established a profile consistent with the following subclasses: Proneural, U3013MG, U3047MG, U3065MG; Mesenchymal U3024MG, U3037MG, U3054MG; Classical U3017MG, U3031MG, U3086MG. The 63 compounds were examined in a recovery assay, by quantification of cytotoxicity, apoptosis and cell viability in U3013MG GCSs and human fibroblast cells, as well as cell cycle analysis by FACS. The recovery assay was performed by a two-day incubation of compounds at different concentrations followed by two more days without compound. While 25 compounds had a reversible and 38 a permanent effect, only three compounds (including S10) had an irreversible effect at the same concentration that caused the acute effects.

### Selectivity and Efficacy Analysis

To assess the selectivity of compounds on mixed cultures consisting of GSC with other cell types, a hanging drop-based mixed culture procedure was developed. U3013MG GSCs were labeled with a cell tracker red and fibroblasts with a cell tracker green fluorescent dye for co-cultures to assess selective effects on glioma cells (glioblastoma) in a mixed culture setting. Cells organized in layers in the absence of compounds. Cultures containing the hits at concentration lethal to GSC failed to organize and most led to a marked loss of GSCs with none or minor effects on human fibroblasts following one day incubation with compounds. To measure toxicity, increasing concentrations of the 17 aforementioned hits administered to the water of 10 dpf revealed that while six hits (including S10) did not exert any effect of zebrafish development, the embryos died, decayed or displayed yolk edema in the presence of the remaining hits. These data suggests that S10 selectively and effectively kills glioma cancer cells, in particular glioblastoma cancer cells, or glioma/glioblastoma cancer stem cells in the presence of other cells providing superior selectivity over current therapies.

### Zebrafish In Vivo Efficacy Assay

A xenotransplantation model for GBM in zebrafish was developed to test the capacity of the 17 hits to prevent tumor formation in vivo. Three thousand U3013MG GSCs labeled with cell tracker red were injected intracranially into the ventricle of 48-52 hpf larvae. Each of the 17 hits were administered to the egg water at the lowest effective in vitro cytotoxic concentration identified and tumor development assessed 10 days later. This assay allowed rapid evaluation of the compounds in an in vivo setup, features such as the acute/chronic toxicity effect of the compounds on zebrafish and the transplanted cells, transplanted cell proliferation and migration of cells into brain parenchyma, compounds penetrance into the zebrafish tissue were all parallely evaluated. These features made this xenograft model a powerful tool and reduced the number of compounds that could be taken for evaluation in rodent models. The ease and rapidity to perform this experiment also indicated possibilities to use this assay as a powerful screening tool for identification of compounds active against brain tumors. In this assay, S10 markedly reduced tumor size. Based on these analyses, further studies were focused on compound S10, which we name Vacquinol-1 due to its quinoline-alcohol scaffold. S10 treated GSC displayed high cytotoxicity, led to a complete loss of viability as measured by ATP depletion, and selectively targeted GSCs in mixed co-cultures with human fibroblasts. S10 did not affect ESCs, human fibroblasts or osteosarcoma cells but rapidly reduced the proportion of cells in S and G2/M cell cycle phases. Cardiovascular toxicity was assessed using a recently established model based on frequency spectral analysis of heart beating in ex-vivo adult zebrafish hearts (Kitambi et al., (2012) BMC Physiol. 12, 3). Except for four hits displaying cardiac toxicity, small or no effects were observed on the remaining compounds (including S10). This data suggests that S10 is well tolerated and efficacious in vivo, has no observable cardaic toxicity in zebrafish and selectively kills glioma/glioblastoma cancer cells or glioma/glioblastoma cancer stem cells in an in vivo tumor environment.

### In Vitro Cancer Cell and CSC Viability Assay

The ability of Examples S1-S23 to selectively induce cytotoxicity in glioma/glioblastoma cancer cells or glioma/glioblastoma cancer stem cells was determined by quantification of ATP production in glioma stem cell line U3013 in the presence of using CellTiterGlo reagent (Promega). Cells were exposed to compound in serial dilution in the range 1 nM to 50 M for 24 hours and viability assessed with respect to negative control (dmso, no cell death) and positive control (staurosporine, full cell death). Typically, the efficacy range (EC₅₀) of the evaluated compounds was in the range 0.5-20 M (Table 4). Assessment of viability of GSCs in the presence of S10 in dose-response assays using 3000 cells/cm2 showed a median efficacy concentration of 50% (EC₅₀) at 2.36 μM after 24 hours when compared to the EC₅₀ of 139 μM shown by temozolomide, a commonly used drug for treating glioma/glioblastoma. The EC₅₀ of S10 remained largely similar at 2, 3 and 4 days of incubation. The EC₅₀ of fibroblasts after 24 hrs was 18.7 μM and displayed slightly attenuated EC₅₀ at longer exposure (23 μM at 96 hours). The individual isomers (S21-S23) of racemic S10 were evaluated in order to determine the enantiospecific pharmacology of the individual isomers. Whilst S20 and S21 showed an equal or increased potency with respect to S10, isomers S22 and S23 showed significantly attenuated activity.

TABLE 4
In vitro efficacy (viability)

| Compound | EC₅₀ (μM) |
|---|---|
| S1 | 0.39 |
| S2 | 0.41 |
| S3 | 0.73 |
| S4 | 1.03 |
| S5 | 1.10 |
| S6 | 1.25 |
| S7 | 1.59 |
| S8 | 1.69 |
| S9 | 2.25 |
| S10 | 2.36 |
| S11 | 2.69 |
| S12 | 3.22 |
| S13 | 3.62 |
| S14 | 5.62 |
| S15 | 7.59 |
| S16 | 8.72 |
| S17 | 9.60 |
| S18 | 12.70 |
| S19 | 19.30 |
| S20 | 1.72 |
| S21 | 2.67 |
| S22 | 10.50 |
| S23 | 9.95 |
| S24 | 32.1 |
| S25 | 35.4 |
| S26 | 13.5 |
| S27 | 38.0 |
| S28 | 14.6 |
| S29 | No activity |

These data demonstrate that the evaluated compounds S1-S23 show potent cytotoxic effects against glioma/glioblastoma cancer cells and provide significant improvement versus the current standard therapy (TMZ). In addition, it is shown that the R,S and S,R stereoisomers of S10 (i.e., S20 and S21 respectively) show significantly increased potency against glioma cancer cells in comparison to the S,S and R,R isomers (i.e., S22 and S23 respectively).

### Multiparametric Phenotypic Analysis of Cytotoxicity

A distinctive feature of apoptosis is the rapid loss of ATP associated with decoupling of the respiratory chain. Death of GSCs was therefore examined in the presence of the apoptosis inhibitor Q-VAD. Gating for live and dead cells by FACS analysis revealed that S10 administration (7.5 μM, 7 hrs incubation) led to a marked and significant increase of dead cells, similar to staurosporin (1 μM, 7 hrs incubation). However, Q-VAD only modestly rescued S10 treated cells from death at 3 and 7 hrs. Staining for active cleaved Caspase-3 in cultures with 7.5 or 15 μM of S10 did not reveal any increased number of immunoreactive cells, as compared to vehicle (DMSO) treated cultures, while doxorubicin (10 μM) caused a marked increase of positive cells. Caspase-3 and Caspase-7 enzymatic activity was measured at 2, 15, 30, 60, 120, 120, 240, 360 and 600 minutes after addition of S10 at increasing concentrations from 5-30 μM. Unlike staurosporin, which within 60 minutes caused a rapid increased activity, S10 had no effect on caspase activity at any concentration or time-point relative to DMSO control. The rapid depletion of ATP by S10 led us to therefore examine the mitochondria. The accumulation of tetramethylrhodamine ethyl ester (TMRE) in mitochondria and the endoplasmic reticulum is driven by their membrane potential. TMRE incorporation in mitochondria was largely unaffected by S10. These results show that S10-induced GSC death occurs by a nonapoptotic mechanism and does not involve a disruption of active mitochondria. Using ratiometric calcium imaging with ATP administration as positive control, cytosolic calcium flux was found not to be affected by S10.

To examine if death involved formation of authophagosomes, immunofluorescence staining of S10 stimulated cells were carried out with an antibody against an established autophagosome marker, microtubule-associated protein light chain 3 (LC3). S10 administration did not lead to any increase of immunoreactivity and remained similar to control cells with only small punctate structures. This suggests that autophagic cell activity likely is not elevated or inhibited by S10 in glioma/glioblastoma cells. Scanning electron microscopy on S10-treated GSC revealed a rapid rounding of cells and appearance of membrane invaginations curved into crater-like cups on the cell surface membrane, indicating an endocytic-like activity. Consistently, live cell imaging at high magnification revealed the formation of spherical protrusions, blebs, appearing within seconds of exposing the cells to S10. With standard phase contrast optics, live imaging revealed within minutes of S10 exposure (15 μM), cell rounding and the formation of massive membrane ruffles and eventual death of cells by a rupture of the cytoplasmic membrane, preceded by a marked contraction of the cytoplasmic membrane followed by uncontrolled expansion resulting in its rupture. Live imaging with Nomarski (interference contrast) optics showed a rapid formation of intracellular vacuoles and membrane invaginations within 10 minutes following S10 at 3.5 μM, with a dose-dependent increase of vacuole formation. Vacuole size and numbers increased with time and led to displacement of the cytoplasm with large vacuoles and eventually cell rupture. These results confirm an induction of endocytic-like activity by S10.

Using cellular imaging, the large vacuoles of varying sizes were clearly observed as lucent, a characteristic of vacuoles resulting from macropinocytosis. Another unique feature of macropinocytosis is a large nonselective internalization of fluid trapped beneath the projections of plasma membrane during membrane ruffling (Schmidt et al. (2011) EMBO J, 30, 3647-3661; Watts and Marsh (1992) J Cell Sci, 103, 1-8). Hence, rapid incorporation of extracellular-phase fluid tracers is a hallmark of macropinosomes. The addition of Lucifer Yellow (LY) to the medium in the presence of S10 led to incorporation of the tracer in most or all cells within 20 minutes with an appearance of the tracer within vacuoles. Internalization of LY was observed occasionally in non-stimulated GSCs, but at a very low rate compared to S10-treated cells. Fluid phase tracers can also enter the early clathrin-coated endosomes, while macropinocytosis is a clathrin-independent process. Clathrin-independent endocytosis of the macropinocytosis type is sensitive to the specific inhibitor of the vacuolar-type H+-ATPase, Bafilomycin A1 (Baf-A) (Bhanot et al. (2010) Mol Cancer Res, 8, 1358-1374; Kaul et al. (2007) Cell Signal, 19, 1034-1043; Overmeyer et al. (2011) Mol Cancer, 10, 69). A short-term (1 h) incubation of GSCs with 100 nM Baf-A had no effect by itself on uptake of LY, but completely abrogated S10-induced LY uptake.

Macropinocytosis is also sensitive to perturbation of the activity of PI3K by Wortmannin (Lehner et al. (2000) Curr Biol, 10, 839-842), Dynamin by dynasore (Gold et al. (2010) PloS One, 5, e11360) and actin by Cytochalasin D (Grimmer et al. (2002) J Cell Sci, 115, 2953-2962) which all completely prevented S10-induced LY uptake in GCSs.

Transmission electron microscopy (TEM) performed on GCSs exposed to S10 for 6 hrs at 7.5 uM concentration confirmed quantitatively induction of a massive vacuolization in cells. Clathrin-coated endosomes are regular in size and bounded by double membrane. The numerous vacuoles observed in GSCs were large, mostly empty and bounded by a single membrane, and displayed an absence of cytoplasmic coats, features consistent with macropinosomes (Overmeyer et al. (2008) Mol Cancer Res, 6, 965-977). The lucent vacuoles induced by S10 were distinct from lysosomes, autolysosomes and late endosomes, which typically contain electron dense organelle remnants or degraded cytoplasmic components (Dunn (1990) J Cell Biol, 110, 1935-1945; Overmeyer et al. (2008) Mol Cancer Res, 6, 965-977). Swollen endoplasmic reticulum and mitochondria and distorted bilayer structures of nuclear membrane were occasionally observed, suggesting occasional aberrant membrane fusion of vacuoles. In cells on the verge of lysis, the vacuoles had typically expanded to a point where much of the cytoplasmic membrane was disrupted. Macropinosomes display a varying size ranging from approximately 0.5-5.0 m consistent with the range of S10 induced vacuoles quantified by TEM (7.5 μM concentration, 6 hrs). Despite being lucent vacuoles and separated from lysosomes, macropinocytic vacuoles recruit the late endosomal and lysosomal marker LAMP1 (Overmeyer et al., (2008) Mol Cancer Res, 6, 965-977). Consistently, S10 (7.5 μM) led to a rapid and marked increase of LAMP1 immunofluorescence in GSCs after 6 hrs of stimulation, that occasionally also was associated with membrane protrusions.

These results collectively provide evidence for initiation of massive macropinocytosis by S10 leading to catastrophic vacuolization resulting in a necrotic-like cell death.

shRNA Screen for Identification of Implicated Cellular Pathways

A genome wide screen with shRNA libraries was used to identify pathways for S10 induced macropinocytosis. The approach was based on the idea that depleting a key factor in the pathway should render GSCs refractive to S10 induced death. Three different DECIPHER pooled lentiviral shRNA libraries consisting of 82500 shRNA covering 15377 genes grouped into Human Module 1 (genes associated with various signaling pathways), Human Module 2 (disease-associated genes) and Human Module 3 (genes associated with cell surface, extracellular and DNA binding), were used to transduce U3013MG GSCs. Four days later, 14 μM S10 was added for one day after which cells were cultured in standard medium for five month. Surviving cells were thereafter dissociated and further expanded. Surviving cells displayed markedly different cell appearance and had lost their elongated morphology with cell protrusions and instead were small and rounded. The resulting S10-resistant GSCs displayed an EC₅₀ of 14.3±1.16 μM on GSC viability, similar to fibroblasts (EC₅₀ of 18.7±0.06 μM). Sequencing of DNA prepared from the resistant GSCs revealed a marked enrichment of presence of a MAP2K4 shRNA virus. Fluorescence staining and western blot analyses of GSCs for activating phosphorylation of MKK4 encoded by MAP2K4, revealed a rapid and pronounced activation by S10. Phospho-MKK4 increased within 5 min of S10 exposure and remained at similar levels for at least 26 hrs of stimulation. Abrogation of MAP2K4 activity by five independent shRNAs led to marked increase of the EC₅₀ viability value of S10-treated GSC. Immunostaining for phospho-MKK4 revealed a punctate cytoplasmic staining in S10-treated cells. These results identify activation of MKK4 as a critical node in the signaling pathway executing S10 induced death of GSCs. MKK4 was thereafter confirmed as a required protein for S10 induced macropinocytosis. Thus, following knock-down of MAP2K4 S10 failed to induce vacuolization as well as LY incorporation, similar to that seen with osteosarcoma cells, showing that resistance to death is associated with a defective formation of macropinosomes induced by S10. Thus, the distinctive feature of susceptibility to macropinocytosis and death in GSCs require MKK4 activity.

### SAR Analysis

Compounds were tested in a standard 11-point dose-response assay measuring viability through luminescence-based ATP quantification, revealing key regiochemical and stereocemical features critical for efficacy (see Table 1 and 2).

### Example 14

### Attenuation of In Vivo Tumor Growth and Infiltration by S10

In vivo pharmacokinetic analysis of plasma and brain exposure following iv, ip and per oral administration revealed a long half-life and excellent bioavailability. The zebrafish xenografts glioblastoma model was developed for quantitative analyses on the efficacy of S10 to inhibit tumor development and for quantification of infiltration of cells in the host brain. Fluorescently labeled U3013MG GSCs were injected intracranially into the ventricle of 48-52hpf zebrafish larvae. Within one week, the GSCs rapidly expanded and formed a tumor cell mass within the ventricle and started to infiltrate the brain. The developing tumors were confirmed to be of human origin by staining for human nuclear antigen. GSC grafted zebrafish were treated with S10 (15 μM) applied to the aquarium water for 10 days. The size of the tumor was determined by quantification of the area, fluorescence level and infiltration by measuring the average distance of infiltrating cells from the original tumor mass. S10 treated animals showed a marked attenuation of tumor growth. Furthermore, cell migration into the brain parenchyma was reduced, indicating effects on tumor infiltration.

The ability of S10 to attenuate tumor progression was next examined in a mouse model for human GBM. Nod/SCID mice received intracranial injections of 100 000 U3013M GSCs and the resulting tumor was allowed to develop for 7 weeks. All mice presented with large and highly vascularized tumors infiltrating the host brain and often displayed massive areas of necrosis, overtly observed during dissection of the brains. Histopathologic analysis of the tumors showed several features of glioblastoma multiforme including areas of pseudopalisading necrosis, mitotic cells and extensive microvascular proliferation. Tumors were highly immunoreactive for human Nestin (hNestin) and human GFAP (hGFAP). S10 (15 μM, 0.5 μL/hr) or vehicle (DMSO) was administered into the site of original cell deposit by an osmotic minipump 6 weeks after cells were grafted. Animals were collected for histological analysis following one week of treatment. Despite the advanced stage of cancer at the time of initiation of S10 administration, the loss of brain tissue by necrosis was markedly and significantly reduced in animals treated with S10 as compared to vehicle and the tumors were invariantly smaller. Consistently, tumor infiltration and area of hGFAP and hNestin immunoreactivity was significantly reduced in S10 treated animals. Tumors in S10-treated mice were not circumscribed with well-defined boundaries, indicating that S10 halted tumor growth and reduced the density of remaining glioblastoma cells both within the tumor mass and around the boundaries. A massive LAMP1 staining was observed within the tumor cell mass following one week S10 administration, with most or all cells displaying immunoreactivity while mice receiving vehicle were devoid of LAMP1. These results show that S10 activates similar pathway in vivo as in vitro, that activation of this pathway is selective for GBM as no staining was observed in the host brain and that it has the capacity when administered to attenuate tumor growth.

The bioavailability of S10 by oral and intraperitoneal injection in vivo was investigated. In vivo pharmacokinetic bioanalysis of plasma and brain exposure following iv, ip and per oral administration revealed a long half-life (t_{1/2}=20 hrs) and excellent bioavailability (F=69%). In a second delivery regimen, treatment was performed per orally (20 mg/kg) twice daily for five days. Treatment started at a terminal stage of GBM, i.e. six weeks after engraftment of U3013M GSCs and median survival from the time of treatment initiation and the Hazard ratio were scored. S10 treated animal showed a median survival of 12 days (n=9 animals) when compared to 7 days (n=9 animals) in DMSO treated animals (95% CI ratio between 0.2109-0.9557). Comparison of the two survival curves indicated a Hazard ratio of 2.293, indicating that the rate of death in the untreated group was more than twice that of the S10 treated group.

These results indicate that S10 is well tolerated in vivo, has a favourable pharmacokinetic profile, and extends life expectancy even at terminal stages of GBM in a mouse xenograft model.

### Cell Culture

GSCs were grown in serum-free media supplemented with N2, B27, EGF, and FGF-2 (20 ng/ml) using previously described methodology (Sun 2008). Culture plates were pre-coated with Laminin (Sigma) for 3 hr at 10 ug/ml prior to use and confluent cells were split 1:3 to 1:5 using TrypLE Express (Invitrogen). Human osteosarcoma and fibroblast cell lines were cultured in DMEM medium (Invitrogen, USA) supplemented with 10% FBS (Invitrogen, USA) as previously described (Bruserud et al. (2005) J Cancer Res Clin Oncol, 131, 377-384; Hovatta et al. (2003) Hum Reprod, 18, 1404-1409). R1 mESCs were cultured in DMEM/F12 supplemented with N2 supplement, 0.4 mM 2-mercaptoethanol, 5 mM HEPES (all from Invitrogen), 10 ng/mL basic fibroblast growth factor and 1,000 U/ml ESGRO (Chemicon) in suspension as previously described (Andang et al. (2008) Nature, 451, 460-464). Cells were dissociated with trypsinization (Tryple E™ Express 1×, Gibco). For experiments, mESCs were grown on 0.2% gelatin coated plates. For primary mice glia cell culture, neonatal mice at PO stage were taken and the brain tissue dissected and cultured as per published protocol (Tamashiro et al. (2012) J Vis Exp, e3814).

### Animal Maintenance and Tissue Collection

All animal work was performed in accordance with the national guidelines and local ethical committee Stockholms Djurförsöksetiska Nämnd. Wildtype C57 male mice and NOD-SCID mice (Charles Rivers) were spaciously housed and experiments were performed according to approved protocols. Perfusion and fixation were performed as previously described (Deferrari et al. (2003) Diabetes Metab Res Rev, 19, 101-114; Phiel et al. (2003) Nature, 423, 435-439). Brain was dissected out of the perfused mice and transferred into 4% PFA in PBS overnight at 4° C. Wild type zebrafish were maintained at 28.5° C. and under standard conditions of feeding, care and egg collection. Embryos were collected by natural mating and staged according to Kimmel et al. (Kimmel et al. (1995) Dev Dyn, 203, 253-310). Embryos were staged in hours post fertilization (hpf) and days post fertilization (dpf), the collected embryos were first anesthetized using 0.1% Tricane, kept on ice and fixed at different stages in 4% paraformaldehyde overnight, then washed with phosphate buffered saline containing 0.1% Tween-20 (PBSTw).

### Small Molecule Screening Setup and Phenotype Analyses

The NCI Diversity Set II small molecule library was analyzed in silico using JChem for Excel (ChemAxon) software to identify and group 1364 small molecules in regards to amenable chemistry and structural compatibility for biological testing. The identified subset was then obtained as 10 mM DMSO stock solution from the NCI/DTP Open Chemical Repository (http://dtp.nci.nih.gov/). For primary screening, 96 well clear-bottom microtiter plates (Corning) were either pre-coated with laminin (Sigma) for 3 hrs prior use for screening on GSCs, or were coated with 0.2% gelatin (Sigma) 3 hrs prior use for mESC, or were washed once with sterile 1×PBS (Invitrogen) 30 min prior use for fibroblast, osteocarcoma or primary mouse glia cells. Prior to screening, laminin or gelatin or PBS was removed from the 96 well plate and cells diluted to an amount of 10,000 cells in 100 μl of respective media per well. Cells were dispensed into each well and incubated overnight. Wells at the outer circumference of the plate were not taken for screening and served as controls for each lane. Primary screening was performed on GSC (U3013 and U3047), fibroblast and mESCs at two concentrations (5 μM and 30 μM). Compounds were manually pipetted into each well and GSCs or fibroblast or osteosarcoma or mouse glia cells were incubated for 24 hrs following which the cells were fixed using 4% paraformaldehyde (PFA). For mESC screening, cells were grown for 4 days and allowed to form colonies. Fixed cells were washed with PBS twice and incubated for 30 min with Phalloidin and DAPI solution in PBS according to the manufacturer instruction. Following incubation, cells were washed with PBS twice and imaged using a Zeiss Axiovert inverted microscope equipped with a CCD camera. The images were then grouped into three categories, normal (similar to untreated or DMSO treated), Loose or Fused (cells were more amoebic in shape and formed aggregates) and Tiny (dead cell with ruptured cytoplasm and/or dramatically reduced size). Selected wells representing each category were taken for confocal imaging. For mESC, brightfield images of colonies were obtained with the above setup after 4 days of culture with or without compound. The images of mESCs were grouped into Live (phenotypically normal ESC colonies) or Dead (single mESC cells which were either dead or failed to form colonies). From the primary screen, the effect of each molecule tested was documented and compared between GSC (U3013, U3047) and with mESCs and fibroblast cells to identify compounds affecting only GSC. The identified compounds were then exposed to a panel of other GSCs lines (U3013, U3047, U3024, U3031, U3037, U3086, U3054, U3065) and the effect was documented.

For treatment with various inhibitors of macropinocytosis, GSC were first preincubated for 30 minutes with the inhibitors and then S10 was added and incubated for approximately 5 hrs following which leucifer yellow (LY) was added and incubated for 20 min. The media was then washed away and fresh media was replaced and the plate was take for imaging. The percent of cells with LY was scored and graph plotted with that data.

### Multiparametric Assays

For measuring cell viability, cytotoxicity and apoptosis in GSC/fibroblast/mGlia cells were grown in 384-well microtiter plates using procedure described above. A total of 10,000 cells was distributed per well and incubated overnight in 45 μl of their respective growth media. Test compounds were then transferred into the well to a final volume of 50 μl and the plates were further incubated for 24, 48, 72 or 96 hrs respectively. Cell viability, cytotoxcity and apoptosis were measured using CellTiter-Glo® Luminescent Cell Viability Assay (Promega), CytoTox-Glo Cytotoxicity Assay and Caspase-Glo 9 Assay, according to manufacturer's instructions. To measure time-dependent release of caspase 3 and 7, a 96-well PP microtiter compound plate (NUNC) was prepared to give 20 μl/well of a continuous 11-point dose-response dilution from 3 mM-500 μM compound in 100% DMSO in column 1-11 of each row. Negative (100% DMSO) and positive (1 mM staurosporine in DMSO) controls was placed in rows 1-4 and 5-8 of column 12, respectively. The plate was diluted with 180 μl growth media/well using a FlexDrop (Perkin Elmer) and 5 μl of the resulting compound solution was transferred in quadruplicate at increasing time-points (5 min, 15 min, 30 min, 60 min, 120 min, 240 min, 360 min, 600 min) to a 384-well black clear-bottom microtiter plate with GSC grown to 70% confluency in 45 μl media per well as described above using a CyBiWell (CyBio Systems) with a 96-well pipetting head, followed by incubation. After the final time of compound addition, the plate was removed from the incubator, and freshly prepared CaspaseGlo (Promega) reagent was added to each well of the plate according to the manufacturer's recommendations. Luminescence was measured using a Victor3 FA (Perkin Elmer) microtiterplate reader and the level of released Caspase 3/7 quantified relative to control using GraphPad Prism (v6.02) software.

To determine compound dose-response inhibition of GSC viability and determine induction of vacuolization, a 96-well PP (NUNC) compound plate was prepared as described above resulting in a serial dilution of each compound from 10 mM to 0.17 uM in 100% DMSO in columns 1-11 (10 μl/well). Negative (100% DMSO) and positive (10 mM S10 in 100% DMSO) controls were placed in rows 1-4 and 5-8, respectively, of column 12. The wells were diluted with 190 μl of the corresponding growth media and 5 μl of each well of compound solution transferred to quadruplicate wells of a sterile 384-well black clear bottom plate (BD Falcon) containing GSC at 70% confluency in 45 μl growth media. The plate was incubated for 24 hours, after which the plate was removed from the incubator and each well imaged in bright-field using an Operetta Imaging system (PerkinElmer) at 37° C. and 5% CO2 to determine vacuole accumulation at each concentration. The plate was then allowed to cool to room temperature and each well treated with 25 μl freshly CellTiterGlo (Promega) reagent. The plate was shaken for 15 minutes and luminescence measured using a Victor3 (Perkin Elmer) microtiterplate reader. Total luminescence was normalized relative to control and curve fitting performed using GraphPad Prism (v6.02) software.

To perform the mixed culture assay, cells were separately labeled with Cell Tracker Red (Invitrogen) or Cell Tracker Green (Invitrogen) one hr prior to use as per manufacturer's instructions. The labeled cells were then washed twice with PBS and resuspended in their respective media. A total of 2000 cells (1000 labeled red and 1000 labeled green) were pipetted onto a drop measuring a final volume of 50 μl of media (with or without compound) on the lid of the petri plate. The lid was then carefully overturned onto the 10 cm petri plate containing 20 ml of PBS. The plates were incubated overnight, following which the cells were fixed using 50 μl of 8% PFA to make a final concentration of 4% PFA. The fixed cells were immediately transferred into a glass bottom petri dish (Corning) and immediately taken for confocal imaging.

For performing the dilution and recovery assay, GSCs were dissociated and distributed into 96 well plates as for the screening. Compounds producing a phenotype from the primary screen were added and the plates incubated for two days. The produced phenotype was recorded following which, the compound containing media was removed, the cells washed twice with PBS and fresh growth media without compound was added and plates incubated for 2 days. The cells were thereafter fixed and stained with phalloiding and DAPI as described above and the phenotype recorded. For FACS-based cell cycle profiling, GSCswere grown to 70% confluence and exposed to either DMSO or compounds at the indicated concentrations overnight followed by dissociation and resuspension in 1 ml of PBS. Cells were then fixed overnight in 75% ethanol and rehydrated in PBS following which propidium iodide (PI) (Roche) staining was performed as described earlier (Anding et al. (2008) Nature, 451, 460-464). Flow cytometry was performed on a FACScan instrument using CellQuest Pro software and analyzed with FlowJo software (Tree Star, Ashland, Oreg., USA). The percentage of apoptotic and dead GSCs were quantified by double staining with Annexin V and propidium iodide (PI) (Roche) and data acquired by flow cytometry. GSCs were treated with DMSO, S10 or Staurosporin and trypsinized after treatment, then suspended in 100 μl incubation buffer, 2 μl Annexin V and 2 μl PI and kept in the dark for 10 min at room temperature. The cells were analyzed by flow cytometry within one hour. Flow cytometry was performed on a FACScan instrument using CellQuest Pro software and analyzed with FlowJo software (Tree Star, Ashland, Oreg., USA).

Ratiometric calcium imaging and quantification was conducted by loading cells with Fura-2/AM (Molecular Probes, Leiden, The Netherlands) and Ca2+ imaging was performed according to (Usoskin et al. (2010) PNAS, 107, 16336-16341), except that the final Fura-2/AM concentration was 1 μM and experiment was run at 37° C. in Krebs buffer. DR/Ro=(R−Ro)/Ro was calculated to measure cellular response, where R is F340/F380 ratio and Ro is a baseline ratio before each stimulus onset (average of three data points preceding stimulations). Ca2+ acquisition rate was 0.1-0.2 Hz between and 1 Hz during stimulation. Compound was applied manually at the lowest concentration that was lethal to GSC. The compounds were applied consequently for 1-2 min with 4-5 minute intervals. Four to five compounds were tested on each plate, followed by ATP stimulation as a positive control at the end of each experiment. The cells were counted as responding to given stimulus if maximum response DRmax/Ro during the course of stimulation exceeded 0.2. Typically, 100 to 150 cells were recorded in one microscope field.

Extracellular fluid uptake was monitored in cells treated for 6 hrs with compound by incubation with Lucifer Yellow (Invitrogen, 1 mg/ml in PBS) for 20 min, two washes with PBS and imaging. Alternatively, Lucifer Yellow was added 15 minutes prior to compound addition and cells incubated for 4-6 hour in the presence of compound before washing and imaging. Images were obtained using a confocal microscope, inverted fluorescent microscope or Operetta (PerkinElmer) cellular imaging system. To visualize active mitochondria and endoplasmic reticulum in cells, TMRE staining (Invitrogen), for visualizing active mitochondria membrane potential and ER tracker (Invitrogen) were used, respectively, according to the directions supplied by the manufacturers.

### In Vivo and Ex Vivo Toxicity Tests

A zebrafish model was used to assess the developmental and cardiac toxicity of advanced hits from the screen. For the developmental toxicity experiment, zebrafish embryos at one-cell stage were distributed into a 96 well plate (3 embryos per well in 200 μl of egg water) and exposed to DMSO as a control or various concentration of compounds. The egg water (with or without compound) was replaced every 6 hrs and the embryos were allowed to grow for three days. The embryos were monitored every day and allowed to grow for 5 days before the phenotype was recorded. For the cardiotoxicity assay, an ex vivo culture of adult hearts was performed according to our previously published procedure (Kitambi et al., (2012) BMC Physiol. 12, 3). Adult hearts from male zebrafish were exposed to compounds and the effect on the heart beat was recorded and analysed using developed methods (Kitambi et al., (2012) BMC Physiol. 12, 3).

### Sectioning

For preparation of frozen cryosections, postfixed mouse brains or zebrafish embryos were transferred to 30% sucrose in PBS and incubated for 2 days at 4° C., after which the sucrose solution was replaced with cryofreeze medium and incubated for 1 day at 4° C. Tissue in cryofreeze medium was then frozen into blocks and sectioned at 14 m on a cryostat. Sections were collected on precoated glass slides as described earlier (Hewitson et al. (2010) Methods Mol Biol, 611, 3-18; Kitambi and Hauptmann (2007) Gene Expr Patterns, 7, 521-528). For paraffin sectioning, isolated brains were fixed and processed for paraffin embedding using standard protocol described elsewhere (Hewitson et al. (2010) Methods Mol Biol, 611, 3-18). Six m thin sections were prepared using a microtome (Ultracut E, Reichert Jung). For preparation of plastic sections, zebrafish embryos were fixed in 4% PFA, dehydrated in 50%, 75%, 85%, and 95% aqueous solutions of ethanol 15 min each, and embedded in JB4 resin (Polysciences, Inc), as described previously (Kitambi and Malicki (2008) Dev Dyn, 237, 3870-3881. Sections, 5 m thick, were prepared using a microtome (Ultracut E, Reichert Jung) and photographed with a digital camera (Axiocam, Zeiss), mounted on a microscope (Axioscope, Zeiss). Images were processed using Photoshop software.

### Histology

For hematoxylin and eosin staining, paraffin sectioned mouse brains were briefly deparaffinized in xylene and hydrated in alcohol gradient till water and stained using Meyer's hematoxylin (cytoplasm) and eosin (for nuclei), then dehydrated in alcohol gradient and cleared in xylene. Permount was used for mounting, as described elsewhere (Fischer et al. (2008) CSH Protoc, 4986). Zebrafish JB4 plastic sections were processed and taken for staining using protocols previously described (Kitambi and Malicki (2008) Dev Dyn, 237, 3870-3881). The stained sections were photographed with a microscope mounted digital camera (Axioscope, Zeiss). Images were processed using Photoshop (Adobe) software.

### Immunostaining

Precoated glass slides with cryosectioned mouse or zebrafish brains were thawed to room temperature and briefly washed with PBS to remove the cryo freeze medium. Mouse brain sections were then processed for either diaminobenzidine (DAB) immunohistochemistry staining or immunofluorescence staining and the zebrafish sections were taken for immunofluorescence staining. The DAB immunostaining procedures were carried out as previously described (Toledo and Inestrosa (2010) Mol Psychiatry, 15, 272-285). Washing and dilution of immunoreagents were carried out using 0.01M PBS with 0.2% Triton X-100 (PBS-T) throughout the experiments. The quenching of endogenous peroxidase activity was achieve with treatment of 0.5% H2O2 for 30 min, followed by incubation with 10% normal donkey serum in PBS-T at room temperature for 1 h to avoid nonspecific binding. Primary antibodies human GFAP (1:500 dilution, Millipore) or human Nestin (1:1000 dilution, Millipore) were incubated overnight at 4° C. Detection was carrying out using biotinylated secondary antibodies (Vector Labs) and developed using ABC amplification (ABC Kit Vector Labs) with 0.6% diaminobenzidine and 0.01% H2O2. After immunostaining, all sections were mounted on superfrost glass slides, air-dried, dehydrated and cover with mounting media D.P.X. (Sigma). For immunofluorescent staining, sections or GSCs grown on coverslip were briefly washed with PBS-T and blocked in 10% normal donkey serum for 30 min (blocking solution). Post blocking, primary antibody solution consisting of anti-LC3 antibody (1:500 dilution, Nanotools) or anti-LAMP1 antibody (1:500 dilution, abcam) or anti-phospho(S257/Thr261)-SEK1/MKK4 (R&D Systems) or human Nestin (1:1000 dilution, Millipore) or anti-human nuclear antigen antibody (1:500 dilution, Chemicon) or anti-activated cleaved caspase 3 antibody (Asp 175) (1:100 dilution, Cell Signaling Technology) in blocking solution as previously described (Marmigere et al., 2006), following which the samples were incubated with flurophore conjugated secondary antibody (Alexa, Molecular Probes) and mounted with immunofluorescence mounting medium (Dako).

### Cell Extracts and Immunoblotting

Whole-cell extracts were prepared in SDS-buffer (25 mM Tris-HCl, pH 7.5, 1 mM EDTA, protease inhibitor cocktail (Roche), and phosphatase inhibitors [2 mM sodium orthovanadate, 20 mM beta-glycerolphosphate], and 1% SDS). The samples were analysed by western blot as described previously (Aranda et al. (2008) Mol Cell Biol, 28, 5899-5911) with the following antibodies: anti-Histone H3 (Abcam), antitrimethyl(Lys27)-Histone H3 (Millipore) and anti-phospho(S257/Thr261)-SEK1/MKK4 (R&D Systems).

### In Silico ADME Prediction

Prediction of drug-likeness, intrinsic aqueous solubility, and passive Caco2 membrane permeability and oral absorption was performed using computational models developed by UDOPP at the Department of Pharmacy, Uppsala University, Sweden. The models are based on carefully curated datasets of drugs and drug like molecules. The solubility and permeability data used to train the models were measured using highly controlled assays that have been developed, optimized and validated at UDOPP during the past two decades.

### Live Imaging

Live imaging of cells was performed in black, clear-bottom, 384-well TC CellCarrier plates (PerkinElmer) using an Operetta High Content imaging system (PerkinElmer) at the indicated magnifications using a live cell chamber kept under 5% CO2 and 37° C. Images and movies were processed using ImageJ software (Rasband, W. S., ImageJ, U. S. National Institutes of Health, Bethesda, Md., USA, http://imagej.nih.gov/ij/, 1997-2012).

### Scanning Electron Microscopy

GSCs grown to 70% confluency were trypsinized and resuspended in 1 ml of growth media containing DMSO or 7.5 μM S10. Cells were exposed to DMSO or 7.5 μM S10 for 6 hrs. The resuspended cells were allowed to drip directly on the surface of a polycarbonate filter (Nuclepore, Inc., Pleasanton, Calif., USA). The polycarbonate filters were specially prepared by GP Plastic AB (Gislaved, Sweden) and supplied by Sempore AB (Stockholm, Sweden). The filter was fitted to an airtight device designed with flow channels, which allowed cells to stream to the center of the filter when vacuum suction was applied from below. When the cell media were completely removed after about two minutes of vacuum suction, they were subsequently coated in a JEOL JFC-1200 Fine Coater (JEOL Tokyo, Japan) for two minutes with ionized gold to a thickness of 40 Å. The total area of each filter with a diameter of 1 cm was examined using a SEM microscope (Philips High Resolution SEM 515, Philips Electronic Instruments, Eindhoven, The Netherlands). The SEM method used in the study has earlier detected human immunodeficiency virus in CSF (Sonnerborg et al. (1989) J Infect Dis, 159, 1037-1041).

### Transmission Electron Microscopy

GSCs were grown to 70% confluency and exposed to either DMSO or 7.5 μM S10 for 6 hrs. Cells were then briefly fixed using 2.5% (wt/vol) glutaraldehyde in 0.1 M phosphate buffer, pH 7.4 at room temperature for 30 min, before being scraped off the petri plate and transferred into an Eppendorf tubes for further fixation and storage at 4° C. Cells were next rinsed in 0.1 M phosphate buffer and centrifuged. Pellets were post fixed in 2% (wt/vol) osmium tetroxide in 0.1 M phosphate buffer (pH 7.4) at 4° C. for 2 h, dehydrated in ethanol followed by acetone, and embedded in LX-112 (Ladd). Ultrathin sections (40-50 nm) were cut using a Leica EM UC 6 ultramicrotome (Leica). Sections were contrasted with uranyl acetate followed by lead citrate and examined in a Tecnai 12 Spirit Bio TWIN transmission electron microscope (FEI) at 100 kV. Digital images were taken using a Veleta camera (Olympus Soft Imaging Solutions). Electron micrographic pictures were obtained as described previously (Ruzzenente et al. (2012) EMBO J, 31, 443-456).

shRNA Screen

GSCs grown to 70% confluency were transduced by DECIPHER pooled lentiviral shRNA libraries consisting of Human Module 1, 2 and 3 using earlier described protocols (Pasini et al., 2008). The successfully transduced cells were then selected using puromycin and replated with growth medium containing DMSO as a control or different concentrations of S10. After 24 hrs of exposure, the DMSO or S10 containing growth medium was replaced with normal growth medium and the cells were allowed to grow until the plates were confluent. The cells were washed and harvested and prepared for genomic DNA extraction and barcode amplification as described earlier (Pasini et al. (2008) Gen Dev, 22, 1345-1355). The amplified bar codes were then taken for sequencing on Illumina Hiseq 2000 sequencer following which statistical analysis of shRNA hits enriched in this screen was done.

### Virus Production, Transduction, and Drug Treatment

The shRNA constructs for MAP2K4 (CLL-H-016251) was obtained from Cellecta. 10 μg of each of the constructs were mixed together with 8 μg of the pCMV-dR8.74psPAX2 packaging plasmid, 4 μg of the VSV-G envelope plasmid and the vectors were transfected into 293FT cells, using the calcium phosphate method (Graham and van der Eb (1973) Virology, 52, 456-467). The lentivirus supernatant was collected 24 h and 48 h post-transfection and filtered through a 0.45 μM low protein binding filter (TPP, Cat. no 99745) to remove debris and 293FT cells. The virus supernatant was concentrated by centrifugation overnight at 4000 g at 4° C. The GSCs were then transduced with the concentrated virus for 48 h with medium containing 4 μg/mL polybrene (Sigma, Cat. no H9268) resulting in approximately 80% transduction efficiency. Next, the virus supernatant was replaced with fresh medium and the transduced cells were maintained for 48 h, allowing expression of the selection marker. Thereafter the cells were split by trypsinization and selected using puromycin (1.5 μg/mL; Life Technologies, Cat. no. A1138-03). After selection, a fraction of the cells were collected for qPCR analysis to test the knockdown efficiency. The remaining cells were maintained in 10 cm tissue culture plates. The transduced cells surviving the drug treatment were split into a 384-well plate for analysis of vacuole formation and ATP synthesis.

### Zebrafish Xenograft Experiment

Zebrafish larvae at 2 dpf (days post fertilization) were anesthetized using tricane using protocol described in the zebrafish book (Westerfield (2000) The zebrafish book, 4th Ed, Eugene, University of Oregon Press). The anesthetized larvae was embedded onto a agarose platform made using larval molds (KLS) and tricane in egg water was filled to keep the embryo under anaesthesia. Glioma (glioblastoma) cells were labeled with Cell Tracker Red as described above and ˜3000 cell were injected per embryo. The embryos were monitored after injection and uninjected or partially injected embryos were removed. The injected embryos were allowed to recover for 30 min in egg water without methylene blue and then transferred into 96 well plates. Three embryos were transferred into each well containing 200 μl of egg water with or without compound. Fresh egg water (with or without compounds) was replenished every 6 hrs for 10 days, following which the embryos were anesthetized and fixed in 4% PFA as described above.

### In Vivo Pharmacokinetics Studies of S10

In vivo pharmacokinetic studies of S10 were performed at SAI Life Sciences Ltd., Hyderabad, India to determine the plasma pharmacokinetics and brain distribution of S10 following a single intravenous, intraperitoneal and oral administration in male BALB/c Mice. Blood samples (approximately 60 μL) were collected from retro-orbital plexus of each mouse. The plasma and brain samples were obtained at 0.08, 0.25, 0.5, 1, 2, 4, 8, 24, 48, 72 and 144 hr (i.v.); 0.08, 0.25, 0.5, 1, 2, 4, 8 and 24 hour (i.p.) and 0.25, 0.5, 1, 2, 4, 6, 8, 24, 48, 72 and 144 hr (p.o.) post dosing. Plasma was harvested by centrifugation of blood and stored at −70° C. until analysis. Immediately after collection of blood, brain samples were collected from each mouse. Tissue samples (brain) were homogenized using ice-cold phosphate buffer saline (pH 7.4) and homogenates were stored below −70° C. until analysis. Total homogenate volume was three times the tissue weight.! Plasma and brain samples were quantified using LC-MS/MS method LLOQ=1.03 ng/mL for plasma and LLOQ=10.25 ng/mL for brain. The plasma and brain concentration-time data for S10 were used for the pharmacokinetic analysis. Brain concentrations were converted to ng/g from ng/mL considering total homogenate volume and brain weight (i.e., dilution factor was 3). Pharmacokinetic analysis was performed using NCA module of Phoenix WinNonlin Enterprise (version 6.3).

### Mouse Xenograft Experiment

GSCs were dissociated with trypsin, resuspended in PBS and kept on ice and the viability of cells were checked using trypan blue before and after the experiment. Surgery in mice was performed using sterile techniques, 6 to 8 week old NOD-SCID mice were anaesthetized using a mixture of isoflurane and oxygen. Mice were positioned onto a stereotaxic apparatus as described elsewhere (Cetin et al. (2006) Nature Prot, 1, 3166-3173) and using a micromotor cordless hand drill (Angthos), a small bore hole was made in the skull above the mouse frontal cortex (coordinates were 1 mm rostral to Bregma, 2 mm lateral to the midline and 2.5 mm deep). A Hamilton microsyringe (10 μl) filled with 100, 000 cells in 5 μl PBS was used to slowly deliver cells into the striatum over a period of 5 min. After the injection procedure, the needle was kept in place for 5 min to minimize reflux of the material and was then removed slowly over a period of 5 min. The bore hole was then filled with bone wax after the operation. For intracerebral dosing, Alzet Micro Osmotic pumps (ALZET M1007D) containing 15 μM S10 in PBS working solution was prepared according to the manufacturers protocol. Osmotic pumps were implanted 6 weeks post cell injection to allow a continuous delivery of S10 to the tumor site for up to 7 days (0.5 μL/hr; 100 μL total volume). After anesthetizing the mice, an incision was made to expose the burr hole previously made for cell injection which was cleaned to remove all bone wax. The pump was inserted and the cannula tip was positioned into the burr hole and glued into place. For tolerance and standardizing oral dosing of S10, wildtype C57 male mice were administered with different doses of S10 (50 mg/kg/day, 40 mg/kg/day, 20 mg/kg/twice daily, 20 mg/kg/day) for one week using standard oral gavage technique. The mice were monitored for weight loss and signs of distress. The dosing regimen indicated 20 mg/kg/day to be well tolerated. NODSCID mice 6 weeks post-GSC injection were thus orally dosed with S10 for 5 days.

### Mouse Kaplan-Meier Experiment

For Kaplan-Meier experiments, 100,000 GSC from U3013MG we injected into NOD-SCID mice as described above. Mice were then monitored for 6 weeks and then oral administration regiment was started. Mice were either given 200 μl of water or S10 in water corresponding to 20 mg/kg, via oral gavage. A total of nine animals were taken for each treatment (control, S10). The oral administration was followed once a day for five days following which the administration was stopped and the animals monitored till they reach the humane end point, after which they were sacrificed.

### EQUIVALENTS

The invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A composition comprising (R)-[2-(4-chlorophenyl)quinolin-4-yl](2S)-piperidin-2-ylmethanol and (S)-[2-(4-chlorophenyl)quinolin-4-yl](2R)-piperidin-2-ylmethanol, further comprising at least one pharmaceutically acceptable excipient, adjuvant, diluent or carrier, wherein the composition comprises less than 1% of (R)-[2-(4-chlorophenyl)quinolin-4-yl](2R)-piperidin-2-ylmethanol and less than 1% of (S)-[2-(4-chlorophenyl)quinolin-4-yl](2S)-piperidin-2-ylmethanol.

2.-3. (canceled)

4. The composition of claim 1 comprising greater than 99% (R)-[2-(4-chlorophenyl)quinolin-4-yl](2 S)-piperidin-2-ylmethanol.

5. (R)-[2-(4-chlorophenyl)quinolin-4-yl](2S)-piperidin-2-ylmethanol.

6.-7. (canceled)

8. The composition of claim 1 comprising greater than 99% (S)-[2-(4-chlorophenyl)quinolin-4-yl](2R)-piperidin-2-ylmethanol.

9. (S)-[2-(4-chlorophenyl)quinolin-4-yl](2R)-piperidin-2-ylmethanol.

10.-11. (canceled)

12. The composition of claim 1 comprising less than 0.1% (S)-[2-(4-chlorophenyl)quinolin-4-yl](2R)-piperidin-2-ylmethanol.

13. The composition of claim 1 comprising less than 0.1% (R)-[2-(4-chlorophenyl)quinolin-4-yl](S)-piperidin-2-ylmethanol.

14. A pharmaceutical composition comprising a composition of claim 1 and a pharmaceutically acceptable carrier or excipient.

15. A method of treating a cancer, comprising administering to a subject a therapeutically effective amount of a composition of claim 1.

16. The method of claim 15, wherein said cancer is associated with altered Ras/Rac activity.

17. The method of claim 16, wherein said cancer is glioma.

18. The method of claim 17, wherein the glioma is glioblastoma.

19. The method of claim 18, wherein the glioblastoma is selected from proneural, classical and mesenchymal glioblastoma.

20.-40. (canceled)

41. A compound selected from
tert-butyl 4-(4-(hydroxy(piperidin-2-yl)methyl)quinolin-2-yl)benzyl(methyl)carbamate,
2-(4-chlorophenyl)-4-(methoxy(piperidin-2-yl)methyl)quinoline,
(2-(4-chlorophenyl)quinolin-4-yl)(pyrrolidin-2-yl)methanol,
(2-(4-ethynylphenyl)quinolin-4-yl)(piperidin-2-yl)methanol,
(2-(4-chlorophenyl)quinolin-4-yl)(1-methylpiperidin-2-yl)methanol,
mixture of 5-(4-((R)-hydroxy((S)-piperidin-2-yl)methyl)quinolin-2-yl)-2-methylbenzonitrile and 5-(4-((S)-hydroxy((R)-piperidin-2-yl)methyl)quinolin-2-yl)-2-methylbenzonitrile,
mixture of 4-(4-((R)-hydroxy((S)-piperidin-2-yl)methyl)quinolin-2-yl)-N,N-dipropylbenzamide and 4-(4-((S)-hydroxy((R)-piperidin-2-yl)methyl)quinolin-2-yl)-N,N-dipropylbenzamide,
mixture of (R)-((S)-piperidin-2-yl)(2-(4-(trifluoromethyl)phenyl)quinolin-4-yl)methanol and (S)-((R)-piperidin-2-yl)(2-(4-(trifluoromethyl)phenyl)quinolin-4-yl)methanol,
mixture of (R)-((S)-piperidin-2-yl)(2-(6-(trifluoromethyl)pyridin-3-yl)quinolin-4-yl)methanol and (S)-((R)-piperidin-2-yl)(2-(6-(trifluoromethyl)pyridin-3-yl)quinolin-4-yl)methanol,
mixture of (R)-((R)-piperidin-2-yl)(2-(4-(trifluoromethyl)phenyl)quinolin-4-yl)methanol and (S)-((S)-piperidin-2-yl)(2-(4-(trifluoromethyl)phenyl)quinolin-4-yl)methanol,
mixture of (R)-((R)-piperidin-2-yl)(2-(6-(trifluoromethyl)pyridin-3-yl)quinolin-4-yl)methanol and (S)-((S)-piperidin-2-yl)(2-(6-(trifluoromethyl)pyridin-3-yl)quinolin-4-yl)methanol,
and a pharmaceutically acceptable salt, solvate or prodrug thereof.

42. The compound of claim 41, wherein the compound is selected from the (R,S) and (S,R) isomers of the aforementioned compounds or the racemic mixture thereof.

43. The compound of claim 41, wherein the compound is selected from the enantiomerically pure (R,S) or (S,R) stereoisomers of the compounds.

44. A pharmaceutical composition comprising a therapeutically effective amount of a compound of claim 41, and at least one pharmaceutically acceptable excipient.

45.-55. (canceled)

56. A method of treating cancer associated with altered Ras/Rac activity in a subject, comprising administering a compound of formula (I) 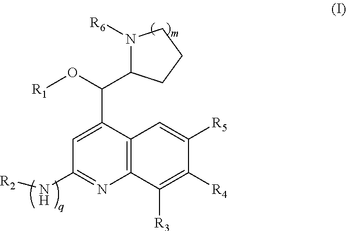
including stereoisomers and tautomers thereof,
wherein
m is 1 or 2;
q is 0 or 1;
R₁ is H or C1-C3 alkyl;
R₂ is selected from C1-C6 alkyl; and C3-C10 unsaturated or saturated, mono- or polycyclic carbocyclyl, heterocyclyl and heteroaryl, each optionally substituted with one or more radicals R₇;
R₃, R₄ and R₅ are independently selected from H, halogen and C1-C6 alkyl optionally substituted with one or more halogens; or
R₃ and R₄, together with the adjacent atoms to which they are attached, form a benzene ring, and R₅ is selected from H, halogen and C1-C6 alkyl optionally substituted with one or more halogens;
R₆ is H or C1-C3 alkyl;
each R₇ is independently selected from C1-C6 alkoxy, C1-C6 alkyl, C1-C6 alkynyl, C1-C6 alkenyl, halogen, alkylamino and NR₈C(O)OR₉;
R₈ is H or C1-C3 alkyl; and
R₉ is C1-C6 alkyl;
or a pharmaceutically acceptable salt, solvate or prodrug thereof,
provided that the compound is not mefloquine, and wherein the cancer is selected from the group consisting of pancreatic, lung, thyroid, urinary tract, colorectal, salivary, prostate, intestinal, skin, hematological/lymphoid malignancies, gliomas and cervical cancer.

57. The method of claim 56, wherein R₂ is C6-C10 unsaturated or saturated, mono- or polycyclic carbocyclyl.

58. The method of claim 56, wherein R₂ is phenyl.

59. The method of claim 56, wherein m is 2 and q is 0.

60. The method of claim 56, wherein the compound is selected from

| Ref. | Structural formula | Formula name |
|---|---|---|
| S1 | 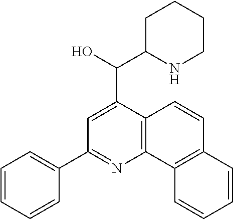 [SMILES: OC(c1cc(-c2ccccc2)nc2c1ccc1ccccc12)C1CCCCN1] | (2-phenylbenzo[h]quinolin-4-yl) (piperidin-2-yl)methanol |
| S2 | 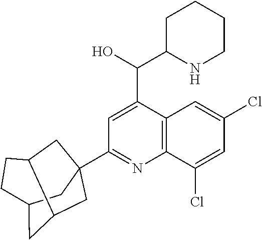 [SMILES: OC(c1cc(C23CC4CCC(C2)C(C4)C3)nc2c(Cl)cc(Cl)cc12)C1CCCCN1] | (6,8-dichloro-2-((2R,3aS,5R)-octahydro- 1H-2,5-methanoinden-2-yl)quinolin-4- yl)(piperidin-2-yl)methanol |
| S7 | 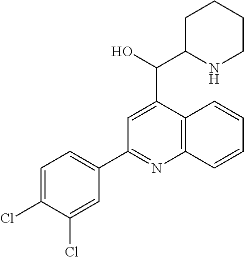 [SMILES: OC(c1cc(-c2ccc(Cl)c(Cl)c2)nc2ccccc12)C1CCCCN1] | (2-(3,4-dichlorophenyl)quinolin-4- yl)(piperidin-2-yl)methanol |
| S8 | 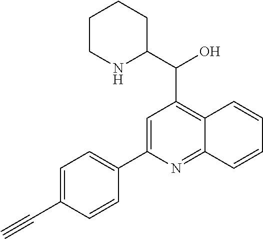 [SMILES: C#Cc1ccc(-c2cc(C(O)C3CCCCN3)c3ccccc3n2)cc1] | (2-(4-ethynylphenyl)quinolin-4- yl)(piperidin-2-yl)methanol |
| S9 | 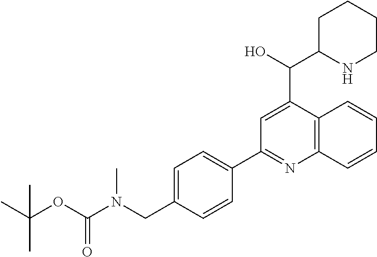 [SMILES: CN(Cc1ccc(-c2cc(C(O)C3CCCCN3)c3ccccc3n2)cc1)C(=O)OC(C)(C)C] | tert-butyl 4-(4-(hydroxy(piperidin-2- yl)methyl)quinolin-2- yl)benzyl(methyl)carbamate. |
| S11 | 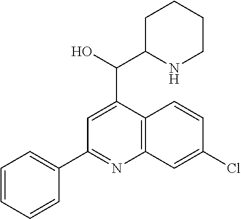 [SMILES: OC(c1cc(-c2ccccc2)nc2cc(Cl)ccc12)C1CCCCN1] | (7-chloro-2-phenylquinolin-4- yl)(piperidin-2-yl)methanol |
| S12 | 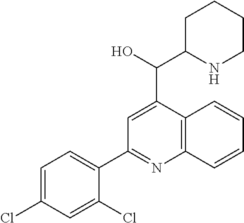 [SMILES: OC(c1cc(-c2ccc(Cl)cc2Cl)nc2ccccc12)C1CCCCN1] | (2-(2,4-dichlorophenyl)quinolin-4-yl)- (piperidin-2-yl)methanol |
| S13 | 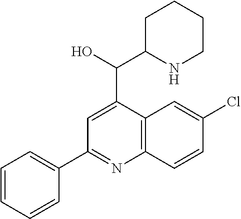 [SMILES: OC(c1cc(-c2ccccc2)nc2ccc(Cl)cc12)C1CCCCN1] | (6-chloro-2-phenylquinolin-4- yl)(piperidin-2-yl)methanol |
| S14 | 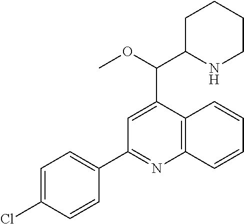 [SMILES: COC(c1cc(-c2ccc(Cl)cc2)nc2ccccc12)C1CCCCN1] | 2-(4-chlorophenyl)-4- (methoxy(piperidin-2- yl)methyl)quinoline |
| S16 | 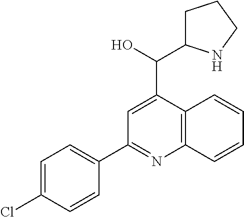 [SMILES: OC(c1cc(-c2ccc(Cl)cc2)nc2ccccc12)C1CCCN1] | (2-(4-chlorophenyl)quinolin-4-yl)- (pyrrolidin-2-yl)methanol |
| S17 | 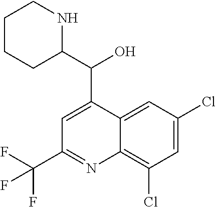 [SMILES: OC(c1cc(C(F)(F)F)nc2c(Cl)cc(Cl)cc12)C1CCCCN1] | (6,8-dichloro-2-(trifluoro- methyl)quinolin-4 yl)(piperidin-2-yl)methanol |
| S19 | 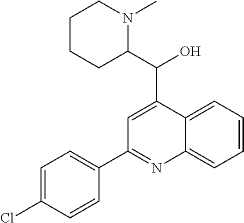 [SMILES: CN1CCCCC1C(O)c1cc(-c2ccc(Cl)cc2)nc2ccccc12] | (2-(4-chlorophenyl)quinolin-4- yl)(1-methyl-piperidin-2-yl)methanol |
| S24 | 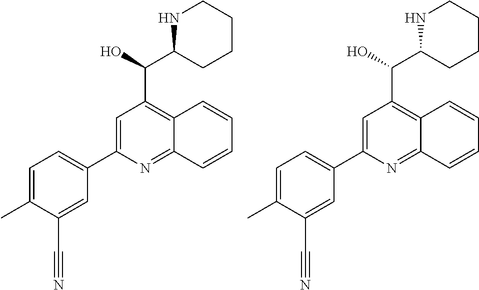 [SMILES: Cc1ccc(-c2cc([C@@H](O)[C@@H]3CCCCN3)c3ccccc3n2)cc1C#N ; Cc1ccc(-c2cc([C@H](O)[C@H]3CCCCN3)c3ccccc3n2)cc1C#N] | Mixture of 5-(4-((R)-hydroxy((S)- piperidin-2-yl)methyl)quinolin-2- yl)-2-methylbenzonitrile and 5-(4-((S)-hydroxy((R)- piperidin-2-yl)methyl)quinolin- 2-yl)-2-methylbenzonitrile |
| S25 | 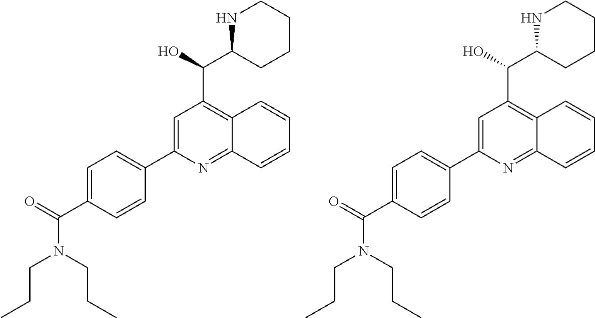 [SMILES: CCCN(CCC)C(=O)c1ccc(-c2cc([C@@H](O)[C@@H]3CCCCN3)c3ccccc3n2)cc1 ; CCCN(CCC)C(=O)c1ccc(-c2cc([C@H](O)[C@H]3CCCCN3)c3ccccc3n2)cc1] | Mixture of 4-(4-((R)-hydroxy((S)- piperidin-2-yl)methyl)quinolin- 2-yl)-N,N-dipropylbenzamide and 4-(4-((S)-hydroxy((R)-piperidin- 2-yl)methyl)quinolin- 2-yl)-N,N-dipropylbenzamide |
| S26 | 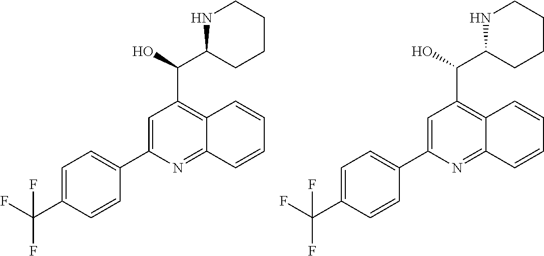 [SMILES: O[C@H](c1cc(-c2ccc(C(F)(F)F)cc2)nc2ccccc12)[C@@H]1CCCCN1 ; O[C@@H](c1cc(-c2ccc(C(F)(F)F)cc2)nc2ccccc12)[C@H]1CCCCN1] | Mixture of (R)-((S)-piperidin-2-yl)(2- (4-(trifluoromethyl)phenyl)quinolin-4- yl)methanol and (S)-((R)-piperidin-2- yl)(2-(4-(trifluoromethyl)phenyl) quinolin-4-yl)methanol |
| S27 | 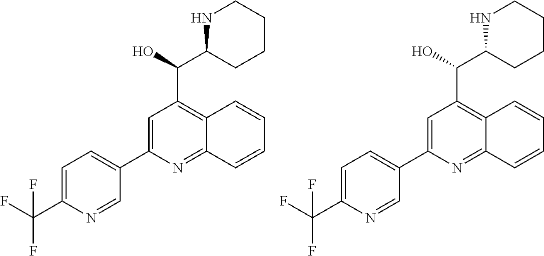 [SMILES: O[C@H](c1cc(-c2ccc(C(F)(F)F)nc2)nc2ccccc12)[C@@H]1CCCCN1 ; O[C@@H](c1cc(-c2ccc(C(F)(F)F)nc2)nc2ccccc12)[C@H]1CCCCN1] | Mixture of (R)-((S)-piperidin-2-yl)(2-(6- (trifluoromethyl)pyridin-3-yl)quinolin-4- yl)methanol and (S)-((R)-piperidin-2- yl)(2-(6-(trifluoromethyl)pyridin- 3-yl)quinolin-4-yl)methanol |
| S28 | 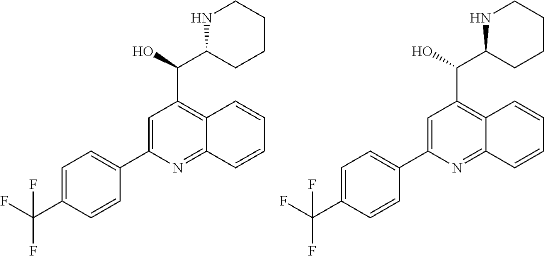 [SMILES: O[C@H](c1cc(-c2ccc(C(F)(F)F)cc2)nc2ccccc12)[C@H]1CCCCN1 ; O[C@@H](c1cc(-c2ccc(C(F)(F)F)cc2)nc2ccccc12)[C@@H]1CCCCN1] | Mixture of (R)-((R)-piperidin-2-yl)(2-(4- (trifluoromethyl)phenyl)quinolin-4- yl)methanol and (S)-((S)-piperidin-2- yl)(2-(4-(trifluoromethyl)phenyl) quinolin-4-yl)methanol |
| S29 | 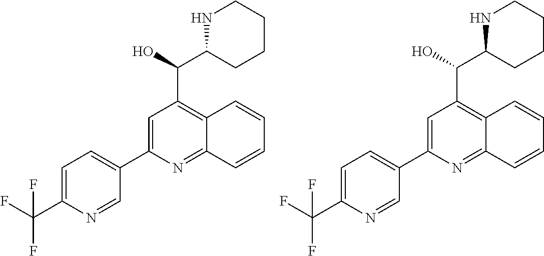 [SMILES: O[C@H](c1cc(-c2ccc(C(F)(F)F)nc2)nc2ccccc12)[C@H]1CCCCN1 ; O[C@@H](c1cc(-c2ccc(C(F)(F)F)nc2)nc2ccccc12)[C@@H]1CCCCN1] | Mixture of (R)-((R)-piperidin-2-yl)(2-(6- (trifluoromethyl)pyridin-3-yl)quinolin-4- yl)methanol and (S)-((S)-piperidin-2- yl)(2-(6-(trifluoromethyl)pyridin-3- yl)quinolin-4-yl)methanol |

and a pharmaceutically acceptable salt, solvate or prodrug thereof.

61. The method of claim 56, wherein, the compound is selected from
tert-butyl 4-(4-(hydroxy(piperidin-2-yl)methyl)quinolin-2-yl)benzyl(methyl)carbamate,
2-(4-chlorophenyl)-4-(methoxy(piperidin-2-yl)methyl)quinoline,
(2-(4-chlorophenyl)quinolin-4-yl)(pyrrolidin-2-yl)methanol,
(2-(4-ethynylphenyl)quinolin-4-yl)(piperidin-2-yl)methanol,
(2-(4-chlorophenyl)quinolin-4-yl)(1-methylpiperidin-2-yl)methanol,
mixture of 5-(4-((R)-hydroxy((S)-piperidin-2-yl)methyl)quinolin-2-yl)-2-methylbenzonitrile and 5-(4-((S)-hydroxy((R)-piperidin-2-yl)methyl)quinolin-2-yl)-2-methylbenzonitrile,
mixture of 4-(4-((R)-hydroxy((S)-piperidin-2-yl)methyl)quinolin-2-yl)-N,N-dipropylbenzamide and 4-(4-((S)-hydroxy((R)-piperidin-2-yl)methyl)quinolin-2-yl)-N,N-dipropylbenzamide,
mixture of (R)-((S)-piperidin-2-yl)(2-(4-(trifluoromethyl)phenyl)quinolin-4-yl)methanol and (S)-((R)-piperidin-2-yl)(2-(4-(trifluoromethyl)phenyl)quinolin-4-yl)methanol,
mixture of (R)-((S)-piperidin-2-yl)(2-(6-(trifluoromethyl)pyridin-3-yl)quinolin-4-yl)methanol and (S)-((R)-piperidin-2-yl)(2-(6-(trifluoromethyl)pyridin-3-yl)quinolin-4-yl)methanol,
mixture of (R)-((R)-piperidin-2-yl)(2-(4-(trifluoromethyl)phenyl)quinolin-4-yl)methanol and (S)-((S)-piperidin-2-yl)(2-(4-(trifluoromethyl)phenyl)quinolin-4-yl)methanol,
mixture of (R)-((R)-piperidin-2-yl)(2-(6-(trifluoromethyl)pyridin-3-yl)quinolin-4 yl)methanol and (S)-((S)-piperidin-2-yl)(2-(6-(trifluoromethyl)pyridin-3-yl)quinolin-4-yl)methanol,
and a pharmaceutically acceptable salt, solvate or prodrug thereof.

62. The method of claim 60, wherein the compound is selected from the (R,S) and (S,R) isomers of the aforementioned compounds or the racemic mixture thereof.

63. The method of claim 60, wherein the compound is selected from the enantiomerically pure (R,S) or (S,R) stereoisomers of the compounds.

64. The method of claim 56, wherein the cancer is glioma.

65. The method of claim 64, wherein the glioma is glioblastoma.

66. The method of claim 65, wherein the glioblastoma is selected from proneural, classical and mesenchymal glioblastoma.

67. A method for selective delivery of a cargo compound, substance or molecule to a cancer cell, comprising
a) covalently conjugating said cargo compound, substance and/or molecule to a composition of claim 1 or a compound of formula (I) 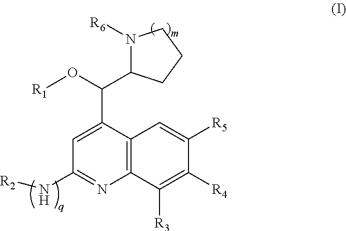
including stereoisomers and tautomers thereof,
wherein
m is 1 or 2;
q is 0 or 1;
R₁ is H or C1-C3 alkyl;
R₂ is selected from C1-C6 alkyl; and C3-C10 unsaturated or saturated, mono- or polycyclic carbocyclyl, heterocyclyl and heteroaryl, each optionally substituted with one or more radicals R₇;
R₃, R₄ and R₅ are independently selected from H, halogen and C1-C6 alkyl optionally substituted with one or more halogens; or
R₃ and R₄, together with the adjacent atoms to which they are attached, form a benzene ring, and R₅ is selected from H, halogen and C1-C6 alkyl optionally substituted with one or more halogens;
R₆ is H or C1-C3 alkyl;
each R₇ is independently selected from C1-C6 alkoxy, C1-C6 alkyl, C1-C6 alkynyl, C1-C6 alkenyl, halogen, alkylamino and NR₈C(O)OR₉;
R₈ is H or C1-C3 alkyl; and
R₉ is C1-C6 alkyl;
or a pharmaceutically acceptable salt, solvate or prodrug thereof,
to form a conjugate
and
b) exposing said conjugate to a cancer cell such that the conjugate contacts the cancer cell.

68. The method of claim 67, wherein the compound of formula 1 is not mefloquine.

69. The method of claim 67, wherein the conjugate contacts the cancer cell in vivo or in vitro.

70. The method of claim 67, wherein the cargo compound, substance or molecule is a cytotoxic compound, a cancer therapeutic or an imaging molecule for selective imaging of cancer cells.

71.-78. (canceled)

79. A screening assay for evaluating a test compound for treating glioma, comprising the steps:
a) preventing pigmentation of zebrafish embryos by
i) injecting embryos at 1 cell stage with a substances that blocks development of pigmentation of embryos,
and/or
ii) adding phenyl thio urea (PTU) to the tank water of an incubator to be used for incubating the embryos
b) placing the embryos in an incubator and allowing the zebrafish embryos to grow for two days post fertilization (2dpf);
c) collecting the zebrafish, and anesthetizing them;
d) injecting unlabelled or dye labelled or transgene expressing cancer cells such as cells from primary tumors of brain tumor glioma cells, such as glioblastoma cells, into the brain ventricle of the embryos;
e) optionally removing wrongly injected embryos
f) allowing the zebrafish to recover from the anaaesthetic, e.g. for about 3-4 hours
g) distributing live swimming zebrafish into a multiwell plate or similar container
h) adding test compounds to the wells or containers at test concentrations
i) exchanging tank water in the wells or containers regularly, such as daily, with water containing said same drug concentration
j) monitoring the zebrafish over time to establish the efficacy of the drug evaluated in the treatment of glioma by determining increase or decrease of glioma (glioblastoma) cells in the zebrafish brain.
